(19) United States
(12) Patent Application Publication
(10) Pub. No.: US 2026/0201039 A1
(43) Pub. Date: Jul. 16, 2026

(54) ACTIVATABLE BISPECIFIC ANTI-CD28 AND ANTI-PD-L1 PROTEINS AND USES THEREOF

(71) Applicant: CENTESSA PHARMACEUTICALS (UK) LIMITED, Altrincham (GB)

(72) Inventor: William James Jonathan FINLAY, Glasgow (GB)

(21) Appl. No.: 19/360,069

(22) Filed: Oct. 16, 2025

Related U.S. Application Data
(63) Continuation of application No. PCT/GB2024/051025, filed on Apr. 19, 2024.
(60) Provisional application No. 63/497,084, filed on Apr. 19, 2023.

(51) Int. Cl. C07K 16/28; A61K 47/68

(52) U.S. Cl. CPC C07K 16/2827; A61K 47/6879; C07K 16/2818; C07K 2317/14; C07K 2317/31; C07K 2317/522; C07K 2317/524; C07K 2317/526; C07K 2317/53; C07K 2317/565; C07K 2317/92

(57) ABSTRACT

Provided herein are protein molecules that specifically bind PD-L1 and also exhibit activatable specific CD28 binding in diseased tissues. Further provided herein are uses of such protein molecules to treat cancer.

### CROSS-REFERENCE

This application is a continuation application of International Application No. PCT/GB2024/051025, filed on Apr. 19, 2024, which claims the benefit of U.S. Provisional Application No. 63/497,084, filed on Apr. 19, 2023, the contents of each of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 19, 2024, is named P90094.WO01_ST26_Sequence_listing.xml and is 90,112 bytes in size.

### TECHNICAL FIELD

The disclosure relates to activatable bispecific proteins and treatments for cancer.

### BACKGROUND

In immune oncology therapy, few of the key drug targets are exclusively expressed in diseased tissue, with the majority also being expressed in non-diseased tissue. In addition, many drugs employed in cancer treatment employ highly potent cell-killing mechanisms of action. As a result, engagement of the target by the drug in non-diseased tissue often causes unwanted side effects.

PD-L1 is a cell surface receptor that is a member of the immunoglobulin superfamily and is principally expressed on myeloid cells and regulatory T (Treg) cells in non-diseased tissues. However, PD-L1 has also been observed to be highly expressed on some cancer cells. PD-L1 binds to the membrane protein PD1. The interaction of PD-L1 with PD1 on T cells down-regulates T cell inflammatory activity, which promotes immune self-tolerance. PD-L1 is, therefore, described as an immune checkpoint. Hence, antagonistic anti-PD-L1 monoclonal antibodies that block interaction with PD1 have demonstrated the potential to act as well-tolerated immunotherapeutic agents in disease settings such as cancer, by liberating anti-cancer T cell responses from natural immune restriction. As such, PD-L1 is a drug target used to amplify the adaptive immune system's anti-cancer effects. In many cases, however, tumors may use multiple ‘escape mechanisms’ to negate the effects of anti-PD-L1 agents, one of which is the downregulation of costimulatory ligand molecules like CD80 (B7.1) and CD86 (B7.2).

CD28 is the receptor for both CD80 and CD86 proteins. It is a glycosylated, disulfide-linked 44 kDa homodimer which is expressed on the cell surface. While CD28 is principally expressed on T cells, it has also been identified on other adaptive and innate immune cells. Binding and activation of CD28 by CD80 or CD86, particularly in conjunction with TCR signaling, provides a powerful pro-immune signal which enhances T cell activation and memory.

Historically, antibodies have been generated against the CD28 extracellular domain which, upon binding, can induce a costimulatory activation signal in T lymphocytes that leads to increased inflammatory activity, proliferation and cytolytic activity against infected or cancerous cells. Using binding domains from these antibodies, CD28-ligating bispecific agents have also been generated that may direct T cell costimulating activity to target-enriched diseased tissues. Anti-PD-L1 antibodies could therefore be made more potent and broadly acting therapeutic agents by gaining the ability to also bind CD28 and thereby strongly enhance T cell killing mechanisms in anti-PD-L1 antibody-resistant disease settings. This would combine a key checkpoint inhibitor function with an inducible ‘synthetic immunity’ that can synergistically stimulate the adaptive immune system. However, the ability to make this combination work in a single therapeutic agent structure (e.g., in standard bispecific antibody format with fully active PD-L1 and CD28 binding domains) is limited by the relatively broad expression profiles of both PD-L1 and CD28 on many cell types such as T cells and myeloid cells, as well as others. This broad expression profile may lead not only to dose-limiting toxicities for PD-L1/CD28 binding agents, but also to profound peripheral sink/biodistribution problems that limit the ability of such agents to achieve high enough exposure in diseased tissues to exploit the combined mechanisms. There is, therefore, a need for engineered forms of bispecific binding proteins with activity specifically targeted to the diseased tissue environment.

### SUMMARY

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD28 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD28 VL domain, and a second immunoglobulin light chain constant region.

In some embodiments, the heavy chain comprises in N-terminus to C-terminus order, the anti-PD-L1 VH domain, the first CH1 domain, the first linker, the anti-CD28 VH domain, the second CH1 domain, a hinge, a CH2 domain, and a CH3 domain.

In some embodiments, the protein further comprises a third polypeptide chain comprising a hinge and a Fc region. In some embodiments, the third polypeptide chain comprises the amino acid sequence of SEQ ID NO: 32.

In some embodiments, the first linker comprises the amino acid sequence of SEQ ID NO: 1, SEQ ID NO: 2, SEQ ID NO: 3, or SEQ ID NO: 4. In some embodiments, the second linker comprises the amino acid sequence of SEQ ID NO: 1, SEQ ID NO: 2, SEQ ID NO: 3, or SEQ ID NO: 4.

In some embodiments, the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14; the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17; the anti-CD28 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and the anti-CD28 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

In some embodiments, the anti-PD-L1 VH domain comprises the amino acid sequence of SEQ ID NO: 24, and the anti-PD-L1 VL domain comprises the amino acid sequence of SEQ ID NO: 25. In some embodiments, the anti-CD28 VH domain comprises the amino acid sequence of SEQ ID NO: 26. In some embodiments, the anti-CD28 VL domain comprises the amino acid sequence of SEQ ID NO: 27.

In some embodiments, the heavy chain comprises the amino acid sequence of SEQ ID NO: 30 or SEQ ID NO: 31. In some embodiments, the light chain comprises the amino acid sequence of SEQ ID NO: 28 or SEQ ID NO: 29.

Provided herein is a protein wherein
(a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
(b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.

In some embodiments, the heavy chain comprises an IgG, IgE, IgM, IgD, IgA, or IgY constant region. In some embodiments, the heavy chain comprises an IgG1, IgG2, IgG3, IgG4, IgAQ1 or IgA2 constant region. In some embodiments, the heavy chain comprises an immunologically inert constant region. In some embodiments, the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.

Provided herein is an immunoconjugate comprising a protein disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising a protein or an immunoconjugate disclosed herein, and a pharmaceutically acceptable carrier, diluent or excipient.

Provided herein is a nucleic acid molecule encoding (a) the heavy chain amino acid sequence; (b) the light chain amino acid sequence; or (c) both the heavy chain and the light chain amino acid sequences of the protein disclosed herein.

Provided herein is an expression vector comprising a nucleic acid molecule disclosed herein.

Provided herein is a recombinant host cell comprising a nucleic acid molecule or an expression vector disclosed herein.

Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Provided herein is a method for enhancing an anti-cancer immune response in a subject, the method comprising administering to the subject a therapeutically effective amount of a protein, an immunoconjugate, or a pharmaceutical composition disclosed herein.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, an immunoconjugate, or a pharmaceutical composition disclosed herein.

In some embodiments of the methods provided herein, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

### BRIEF DESCRIPTION OF THE DRAWINGS

FIG. 1A depicts a diagram of an exemplary asymmetric one-arm construct of the protein molecule disclosed herein in intact (left), activated protease-cleaved (middle) and deactivated protease-cleaved (right) conformations. In an intact conformation, the anti-PD-L1 Fab binding domain is exposed and able to bind their cognate target. The anti-CD28 Fab domain is inhibited from binding by linkers in both the heavy and light chains that are both proteolytically cleavable and may be sequentially cleaved by matrix metalloproteases (MMPs) and/or cathepsins. A first cleavage event creates an intermediate active state which allows both anti-PD-L1 and anti-CD28 Fabs from a single protein construct to bind their cognate targets, thereby potentially directing activation of CD28+ T cells in the PD-L1+ immunosuppressive microenvironment. A second cleavage dissociates the anti-PD-L1 and anti-CD28 Fabs, removing the ability of a single molecule to bind both PD-L1 and CD28 simultaneously. FIG. 1B depicts a symmetric two-arm construct and a symmetric one-arm construct of the protein molecule described herein. FIG. 1B depicts two diagrams. One shows that of an intact symmetric two arm LB (LPD) protein (left) consisting of two heavy chains and two light chains and a diagram of an intact symmetric one arm LB (LPD) protein consisting of heavy chain and light chain (right). Symmetric two arm LPD proteins consist of two light chain fragments, each comprised of two Fabs linked by a lower hinge linker (LHL). Each heavy chain is comprised of two Fabs linked by a lower hinge linker (LHL) an Fc hinge and Fc fragment containing both CH2 and CH3 domains (left). A symmetric one arm LB proteins consist of one light chain molecule that is comprised of two Fabs linked by a lower hinge linker (LHL) and an Fc fragment containing both CH2 and CH3 domains with either N-linked glycosylation stie (CH2) or Knobs mutations (CH2). The light chains is paired with one heavy chain, comprised of two Fabs linked by a lower hinge linker (LHL) an Fc hinge and Fc fragment containing both CH2 and CH3 domains with either N-linked glycosylation sites (CH2) or Knobs mutations (CH3) (right).

FIG. 2 depicts a diagram of the mechanism of activity of activatable bispecific protein molecules provided herein. As the liberated activity of the CD28 binding domain is monomeric in nature, it cannot cause CD28 activation directly and relies upon binding to PD-L1+ cells, leading to polyvalent presentation of CD28 binding in trans to nearby immune cells to induce CD28 signaling. The activated molecule thereby simultaneously blocks PD-L1/PD1 signaling to reinstate uninhibited TCR “Signal 1” and provides CD28 signaling to amplify T cell activity via “Signal 2”.

FIG. 3 depicts surface plasmon resonance (SPR) binding of each CD28 V domain (in monovalent Fab format) to human recombinant CD28.

FIG. 4 shows ELISA binding of purified IgG_001 and IgG_002 or TGN1412 (IgG4) to human/cyno and mouse CD28 protein in ELISA. The negative control protein used is human IgG1 isotype. TGN1412, IgG_001 and IgG_002 bind to similar levels to human/cyno CD28, no signal was detected for isotype controls. No binding for any protein was detected for mouse CD28 recombinant protein.

FIGS. 5A-H depicts MMP12 digested LPD proteins run on SDS PAGE under reducing or non-reducing conditions. 2 μg/lane of exemplary LPB proteins-1, -2, -3, -6, -7, -8, -9, -10, -11, -12 were loaded on reducing/non-reducing SDS-PAGE respectively after incubation with MMP12 for the indicated times. FIG. 5A shows digestion profiles of LPD28-1 and LPD28-2 proteins incubated at 0 h, 1 h, 2 h, 4 h and 8 h with MMP12. Notably, a prominent 25 kDa/50 kDa band in reducing/non-reducing SDS-PAGE respectively appears, resulting from light chain cleavage and disappearing intact heavy chains at 75 kDa in reducing conditions (and 150 kDa in non-reducing conditions). Appearance of the lower band and disappearance of the higher band can be observed in concert, demonstrating that linkers in both the heavy and light chains become cleaved. FIG. 5B shows digestion profiles of LPD28-3 protein incubated at 0 h, 15 min, 30 min, 1 h, 2 h, 4 h and 8 h with MMP12. Notably, a prominent 25 kDa/50 kDa band in reducing/non-reducing SDS-PAGE respectively appears, resulting from light chain cleavage and disappearing intact heavy chains at 75 kDa in reducing conditions (and 150 kDa in non-reducing conditions). Appearance of the lower band and disappearance of the higher band can be observed in concert, demonstrating that linkers in both the heavy and light chains become cleaved. FIG. 5C shows digestion profiles of LPB28-6 incubated for 0 h, 15 min, 30 min, 1 h, 2 h, 4 h and 24 h with MMP12. Notably, prominent 25 kDa and 50 kDa band in reducing SDS-PAGE appear, resulting from light chain and heavy chain cleavage and accompanying disappearing intact chains at 75 kDa in reducing conditions (and 150 kDa in non-reducing conditions). FIG. 5D shows digestion profiles of LPB28-7 incubated for 0 h, 15 min, 30 min, 1 h, 2 h, 4 h and 24 h with MMP12. Notably, prominent 25 kDa and 50 kDa bands in reducing SDS-PAGE appear, resulting from light chain and heavy chain cleavage and accompanying disappearing intact chains at 75 kDa in reducing conditions (and 150 kDa in non-reducing conditions). FIG. 5E shows digestion profiles of LPB28-8 and LPD28-9 incubated for 0 h, 15 min, 30 min, 1 h, 2 h, 4 h and 24 h with MMP12. Notably, prominent 25 kDa and 50 kDa bands in reducing SDS-PAGE appear, resulting from light chain and heavy chain cleavage and accompanying disappearing intact chains at 75 kDa in reducing conditions (and 150 kDa in non-reducing conditions). FIG. 5F shows digestion profiles of LPB28-10 incubated for 0 h, 15 min, 30 min, 1 h, 2 h, 4 h and 8 h with MMP12. Notably, prominent 25 kDa and ~55 kDa bands in reducing and ~50 kD and ~120 kDa in reducing SDS-PAGE, respectively appears, resulting from light chain cleavage and disappearing intact heavy chains at 75 kDa in reducing conditions (and 250 kDa in non-reducing conditions). Appearance of the lower band and disappearance of the higher band can be observed in concert, demonstrating that linkers in both the heavy and light chains become cleaved. FIG. 5G shows digestion profiles of LPB28-11 incubated for 0 h, 15 min, 30 min, 1 h, 2 h, 4 h and 24 h with MMP12. Notably, prominent 25 kDa and ~55 kDa bands in reducing and ~50 kD and ~120 kDa in reducing SDS-PAGE, respectively appears, resulting from light chain cleavage and disappearing intact heavy chains at 75 kDa in reducing conditions (and 250 kDa in non-reducing conditions). Appearance of the lower band and disappearance of the higher band can be observed in concert, demonstrating that linkers in both the heavy and light chains become cleaved. FIG. 5H shows digestion profiles of LPB28-12 and LPD28-13 incubated for 0 h, 15 min, 30 min, 1 h, 2 h, 4 h and 24 h with MMP12. Notably, prominent 25 kDa and ~55 kDa bands in reducing and ~50 kD and ~120 kDa in nonreducing SDS-PAGE, respectively, appear. These are resulting from light chain cleavage and disappearing intact heavy chains at 75 kDa in reducing conditions (and 250 kDa in non-reducing conditions). Appearance of the lower band and disappearance of the higher band can be observed in concert, demonstrating that linkers in both the heavy and light chains become cleaved. Loading mistakes were corrected in reducing gels as depicted by boxes where lanes were moved.

FIG. 6A shows ELISA binding of purified IgG_001 and IgG_002 to human/cyno CD28 protein. Purified IgG_001 and IgG_002 proteins were used as positive controls while human IgG1 isotype was used as negative control. FIG. 6B shows human/cyno CD28 recombinant protein binding of intact or 0 h, 15 min, 30 min, 1 h, 2 h, 4 h and 24 h MMP12 treated LPD28-3. Intact LPD28-3 showed binding at highest concentrations tested only, whereas all MMP12 treated LPD28-3 proteins showed strong engagement for all tested concentrations. FIG. 6C shows human/cyno CD28 recombinant protein binding of intact or 0 h, 15 min, 30 min, 1 h, 2 h, 4 h and 8 h MMP12 treated LPD28-10. Intact LPD28-10 showed binding at highest concentrations tested only, whereas all MMP12 treated LPD28-10 proteins showed strong engagement for all tested concentrations. FIG. 6D shows human/cyno CD28 recombinant protein binding of intact or 0 h, 15 min, 30 min, 1 h, 2 h, 4 h and 24 h MMP12 treated LPD28-11. Intact LPD28-11 showed some level of binding at highest concentrations tested only, whereas all MMP12 treated LPD28-11 proteins showed strong engagement.

FIG. 7A depicts Jurkat cell binding of intact or 0 h, 15 min, 1 h, 8 h MMP12 treated LPD28-3. No binding to Jurkat cells was observed for intact LPD28-3 (0 min) or isotype control. In contrast, all MMP12 treated samples showed similar binding levels to Jurkat cells. FIG. 7B depicts Jurkat cell binding of intact or 0 h, 15 min, 1 h and 8 h MMP12 treated LPD28-10. No binding to Jurkat cells was observed for intact LPD28-10 (0 min) or isotype control. In contrast, all MMP12 treated samples showed similar binding levels to Jurkat cells. FIG. 7C depicts Jurkat cell binding of intact or 0 h, 15 min, 1 h, and 24 h MMP12 treated LPD28-11. Low levels of binding to Jurkat cells was observed for intact LPD28-11 (0 min). No binding was detected for isotype control. In contrast, all MMP12 treated samples showed similar binding levels to Jurkat cells.

FIG. 8 depicts activity of CD28 binding IgG proteins and on Jurkat IL2 reporter cells in presence or absence of CD3 (SP34) stimulation. In the absence of CD3 (SP34) costimulation, luminescence signal was detected for IgG_001 and TGN1412 but not IgG_002. In the presence of CD3 stimulation, both TGN1412 and IgG_002 showed a clear increase in luminescence signal over CD3 only stimulation as represented by the dotted line.

FIGS. 9A-M depict activity of intact or MMP12 treated LPD proteins in the presence or absence of CD3 stimulation and the presence of absence of human PDL1+ MDA-MB-231 cells. FIG. 9A shows activity testing of intact and 2 h MMP12 treated test article LPD28-1 and LPD28-2 in the presence of CD3 stimulation and MDA-MB-231 cells. Intact LPD28-1 showed no activity over CD3 stimulation level while 2 h MMP12 treated LPD28-1 showed clear stimulation of luminescence signals above CD3 signals. In contrast, LPD28-2 treated with MMP12 for 2 h showed no activity over CD3 stimulation level. FIG. 9B showed activity testing of intact and 2 h MMP12 treated test article LPD28-3 in the presence of CD3 stimulation and MDA-MB-231 cells. Intact or MMP12 treated LPD28-3 showed no activity over CD3 stimulation level. FIG. 9C shows activity testing of intact and 2 h MMP12 treated test article LPD28-3 in the absence of CD3 stimulation and presence of MDA-MB-231 cells. Intact or MMP12 treated LPD28-3 showed no activity over background levels. FIG. 9D shows activity testing of intact and 2 h MMP12 treated test article LPD28-3 in the presence of CD3 stimulation but absence of MDA-MB-231 cells. Neither intact nor MMP12 treated LPD28-3 showed activity over CD3 stimulation levels. FIG. 9E shows activity testing of intact and 2 h MMP12 treated test article LPD28-3 in the absence of CD3 stimulation and MDA-MB-231 cells. Neither intact nor MMP12 treated LPD28-3 showed activity over background signals. FIG. 9F shows activity testing of intact and MMP12 treated test article LPD28-8 in the presence of CD3 stimulation and MDA-MB-231 cells. Intact or MMP12 treated LPD28-3 showed no activity over CD3 stimulation level. FIG. 9G shows activity testing of intact and MMP12 treated test article LPD28-8 in the absence of CD3 stimulation and presence of MDA-MB-231 cells. Intact or MMP12 treated LPD28-8 showed no activity over background levels. FIG. 9H shows activity testing of intact and MMP12 treated test article LPD28-8 in the presence of CD3 stimulation but absence of MDA-MB-231 cells. Neither intact nor MMP12 treated LPD28-8 showed activity over CD3 stimulation levels. FIG. 9I shows activity testing of intact and MMP12 treated test article LPD28-8 in the absence of CD3 stimulation and MDA-MB-231 cells. Neither intact nor MMP12 treated LPD28-38 showed activity over background signals. FIG. 9J shows activity testing of intact and MMP12 treated test article LPD28-9 in the presence of CD3 stimulation and MDA-MB-231 cells. Intact or MMP12 treated LPD28-9 showed no activity over CD3 stimulation level. FIG. 9K shows activity testing of intact and MMP12 treated test article LPD28-9 in the absence of CD3 stimulation and presence of MDA-MB-231 cells. Intact or MMP12 treated LPD28-9 showed no activity over background levels. FIG. 9L shows activity testing of intact and MMP12 treated test article LPD28-9 in the presence of CD3 stimulation but absence of MDA-MB-231 cells. Neither intact nor MMP12 treated LPD28-9 showed activity over CD3 stimulation levels. FIG. 9M shows activity testing of intact and MMP12 treated test article LPD28-9 in the absence of CD3 stimulation and MDA-MB-231 cells. Neither intact nor MMP12 treated LPD28-9 showed activity over background signals.

FIG. 10A shows activity testing of intact and MMP12 treated test article LPD28-10 in the presence of CD3 stimulation and MDA-MB-231 cells. Intact LPD28-10 showed no activity over CD3 stimulation level. All MMP12 treated samples showed clear induction of luminescence signals over background signals FIG. 10B shows activity testing of intact and MMP12 treated test article LPD28-10 in the absence of CD3 stimulation and presence of MDA-MB-231 cells. Intact LPD28-10 showed no activity over background levels. All MMP12 treated samples showed clear induction of luminescence signals over background signals. FIG. 10C shows activity testing of intact test article LPD28-10 in the presence of CD3 stimulation but absence of MDA-MB-231 cells. Intact LPD28-10 showed no activity over CD3 stimulation levels. FIG. 10D shows activity testing of intact and MMP12 treated test article LPD28-10 in the absence of CD3 stimulation and MDA-MB-231 cells. Intact LPD28-10 showed no activity over background signals while all MMP12 treated samples show clear activation over background. FIG. 10E shows activity testing of intact and MMP12 treated test article LPD28-11 in the presence of CD3 stimulation and MDA-MB-231 cells. Intact LPD28-11 showed very low activity over CD3 stimulation level at the highest concentration. All MMP12 treated samples showed clear induction of luminescence signals over background signals FIG. 10F shows activity testing of intact and MMP12 treated test article LPD28-11 in the absence of CD3 stimulation and presence of MDA-MB-231 cells. Neither intact LPD28-11 no MMP12 treated LPD28-11 showed activity over background levels. FIG. 10G shows activity testing of intact test article LPD28-11 in the presence of CD3 stimulation but absence of MDA-MB-231 cells. Intact LPD28-11 showed low levels of activity over CD3 stimulation levels at the highest concentrations tested. All MMP12 treated LPD28-11 show activity. FIG. 10H shows activity testing of intact and MMP12 treated test article LPD28-11 in the absence of CD3 stimulation and MDA-MB-231 cells. Neither intact LPD28-11 nor MMP12 treated test articles showed activity over background signals. FIG. 10I shows activity testing of intact and MMP12 treated test article LPD28-12 in the presence of CD3 stimulation and MDA-MB-231 cells. Intact LPD28-12 showed no activity over CD3 stimulation level. All MMP12 treated samples showed clear induction of luminescence signals over background signals FIG. 10J shows activity testing of intact and MMP12 treated test article LPD28-12 in the absence of CD3 stimulation and presence of MDA-MB-231 cells. Intact LPD28-12 showed no activity over background levels. All MMP12 treated samples showed clear induction of luminescence signals over background signals. FIG. 10K shows activity testing of intact test article LPD28-12 in the presence of CD3 stimulation but absence of MDA-MB-231 cells. Intact LPD28-12 showed some activity over CD3 stimulation levels at the highest concentration tested. All MMP12 treated samples showed clear induction of luminescence signals over background signals. FIG. 10L shows activity testing of intact and MMP12 treated test article LPD28-12 in the absence of CD3 stimulation and MDA-MB-231 cells. Intact LPD28-12 showed no activity over background signals while all MMP12 treated samples showed clear activation over background. FIG. 10M shows activity testing of intact and MMP12 treated test article LPD28-13 in the presence of CD3 stimulation and MDA-MB-231 cells. Intact LPD28-13 showed no activity over CD3 stimulation level. All MMP12 treated samples showed clear induction of luminescence signals over background signals FIG. 10N shows activity testing of intact and MMP12 treated test article LPD28-13 in the absence of CD3 stimulation and presence of MDA-MB-231 cells. Intact LPD28-13 showed no activity over background levels. All MMP12 treated samples showed clear induction of luminescence signals over background signals. FIG. 10O shows activity testing of intact test article LPD28-13 in the presence of CD3 stimulation but absence of MDA-MB-231 cells. Intact LPD28-13 showed some activity over CD3 stimulation levels. All MMP12 treated samples showed clear induction of luminescence signals over background signals FIG. 10P shows activity testing of intact and MMP12 treated test article LPD28-13 in the absence of CD3 stimulation and MDA-MB-231 cells. Intact LPD28-13 showed no activity over background signals while all MMP12 treated samples show clear activation over background.

FIGS. 11A-O show activity testing of test articles in a mixed lymphocyte reaction and presence or absence of human PDL1+ MBA-MB-231 cells. LPD28-10 results are shown from 3 different donor pairs. FIG. 11A-B show IL2 release in supernatants of MLR assay in the absence (left) or presence (right) of MDA-MB-231 cells. Neither intact nor MMP12 treat LPD28-1 proteins resulted in increase of IL2 release over background regardless of conditions tested. FIG. 11C shows IFNg release in supernatants of MLR assay in the absence (left) or presence (right) of MDA-MB-231 cells. Neither intact nor MMP12 treat LPD28-1 proteins resulted in increase of IFNg release over background regardless of conditions tested. FIGS. 11D-F show IL2 release in supernatants of MLR assay in the absence of MDA-MB-231 cells. Intact LPD28-10 proteins resulted in no detectable increase of IL2 release over PDL1 blocking (atezolizumab) treatment only. In contrast MMP12 treated LPD28-10 resulted in clear increase of IL2 release over background in the absence of PDL1 engagement. FIGS. 11G-I show IL2 release in supernatants of MLR assay in the presence of MDA-MB-231 cells. Intact LPD28-10 proteins resulted in no detectable increase of IL2 release over PDL1 blocking (atezolizumab) treatment only. In contrast MMP12 treated LPD28-10 resulted in clear increase of IL2 release over background. FIGS. 11J-L show IFNg release in supernatants of MLR assay in the absence of MDA-MB-231 cells. Intact LPD28-10 proteins resulted in some detectable increase of IFNg release over PDL1 blocking (atezolizumab) treatment only. In contrast MMP12 treated LPD28-10 resulted in clear increase of IFNg release over background in the absence of PDL1 engagement. FIGS. 11M-11O shows IFNg release in supernatants of MLR assay in the presence of MDA-MB-231 cells. Intact LPD28-10 proteins resulted in no detectable increase of IFNg release over PDL1 blocking (atezolizumab) treatment only. In contrast MMP12 treated LPD28-10 resulted in clear increase of IFNg release over background.

FIG. 12 depicts primary T cell killing of cancer cell line (MDA-MB-231) mediated purified IgG molecules TGN1412 or IgG_001 and indicated conditions as measured by the Incucyte live cell analysis platform.

FIGS. 13A-D depict primary T cell mediated cell killing of cancer cell line MDA-MB-231 cells by intact or MMP12 incubated LPD proteins with indicated concentrations as measured by the Incucyte® live cell analysis platform. FIG. 13A depicts primary T cell killing of cancer cell line MDA-MB-231 cells mediated by intact or 5 min MMP12 incubated LPD28-1 as measured by the Incucyte® live cell analysis platform. The positive control was a PDL1×CD3 engager for comparison. FIG. 13B depicts primary T cell killing of cancer cell line MDA-MB-231 cells mediated by intact or 15 min or 30 min MMP12 incubated LPD28-10 as measured by the Incucyte® live cell analysis platform. FIG. 13C depicts primary T cell killing of cancer cell line MDA-MB-231 cells mediated by intact or 15 min MMP12 incubated LPD28-12 as measured by the Incucyte® live cell analysis platform. FIG. 13D depicts primary T cell killing of cancer cell line MDA-MB-231 cells mediated by intact or 15 min MMP12 incubated LPD28-13 as measured by the Incucyte® live cell analysis platform.

FIGS. 14A-F depict cytokine release data from PBMC isolated from 20 healthy donors and incubated with intact LPD molecules or control proteins coated on 96 well plates. Incubation was stopped after 72 h and supernatants were tested for cytokines as indicated. FIG. 14A depicts IL2 release stimulated by positive and negative control proteins, buffer (PBS), isotype controls as well as intact LPD molecules. FIG. 14B depicts IFNg release stimulated by positive and negative control proteins, buffer (PBS), isotype controls as well as intact LPD molecules. FIG. 14C depicts IL-10 release stimulated by positive and negative control proteins, buffer (PBS), isotype controls as well as intact LPD molecules. FIG. 14D depicts IL-13 release stimulated by positive and negative control proteins, buffer (PBS), isotype controls as well as intact LPD molecules. FIG. 14E depicts TNFalpha release stimulated by positive and negative control proteins, buffer (PBS), isotype controls as well as intact LPD molecules. FIG. 14F depicts IL-6 release stimulated by positive and negative control proteins, buffer (PBS), isotype controls as well as intact LPD molecules.

FIGS. 15A-B depict cytokine release data from PBMC isolated from 10 healthy donors and incubated with intact LPD molecules or control proteins. LPD28-1 and LPD28-2 were incubated at 3 concentrations (C1=15 μg/mL, C2-2 μg/mL and C3=1 μg/mL). Relevant negative and positive controls were included in the assay such as Blank/Medium (negative control), LPS at 1 concentration (1 μg/mL), commercially available CD28 superagonist at 2 concentrations (15 and 1 μg/mL), Tegenero IgG1 and IgG4 at 2 concentrations (15 and 1 μg/mL). After 24 h, supernatant was harvested and analysed for the secretion of IL-2, IL-6, IL-8, IL-10, IL-13, IFN-γ and TNF-α by Multiplex. FIG. 15A depicts fold changes over buffer control of cytokine release induced by control proteins or intact LPD proteins as indicated. FIG. 15B depicts fold changes over buffer control of cytokine release from RESTORE assay induced by control proteins or intact LPD proteins as indicated.

FIG. 16 depicts the structure of an exemplary symmetric two-arm construct of the protein molecule described herein.

### DETAILED DESCRIPTION

There are two key issues that restrict the efficacy in CD28-activating, tumor-targeting bispecific antibody drugs for oncology:
1) That the antibody target protein found on cancer cells (e.g., PD-L1) is potentially expressed on many different cell classes in the body, not just on the tumor cell. This off-tumor target expression often leads to dose-limiting side effect risks as the CD28 binding domain in a standard bispecific molecule is constitutively active and may therefore direct T cell activation in the presence of any target-positive cell, whether it is in diseased tissue or not. Off-tumor target expression may also lead to antigen “sink” effects, which reduces the amount of drug penetrating the tumor.
2) CD28-positive cells are found in the bloodstream and at high concentration in secondary lymphoid tissues, creating a large sink effect for this arm, affecting biodistribution and free drug availability for tumor penetration.

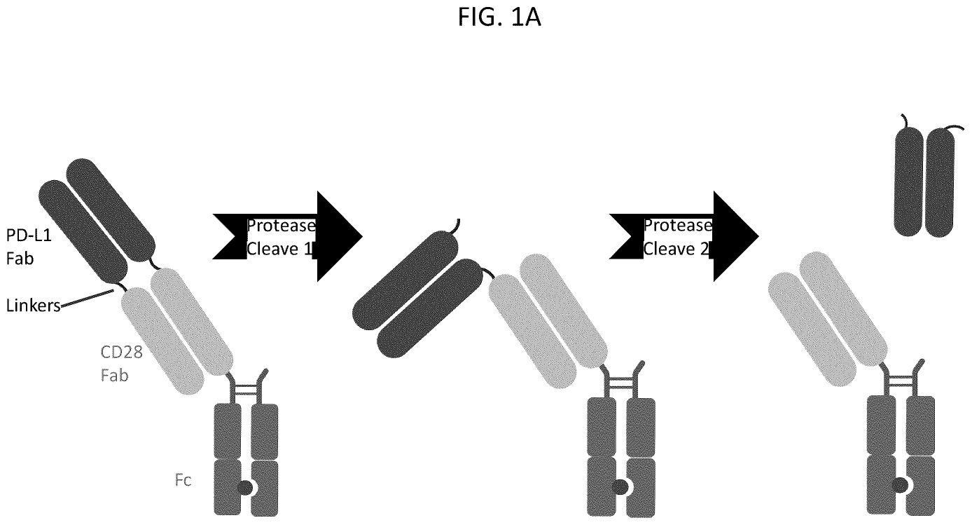

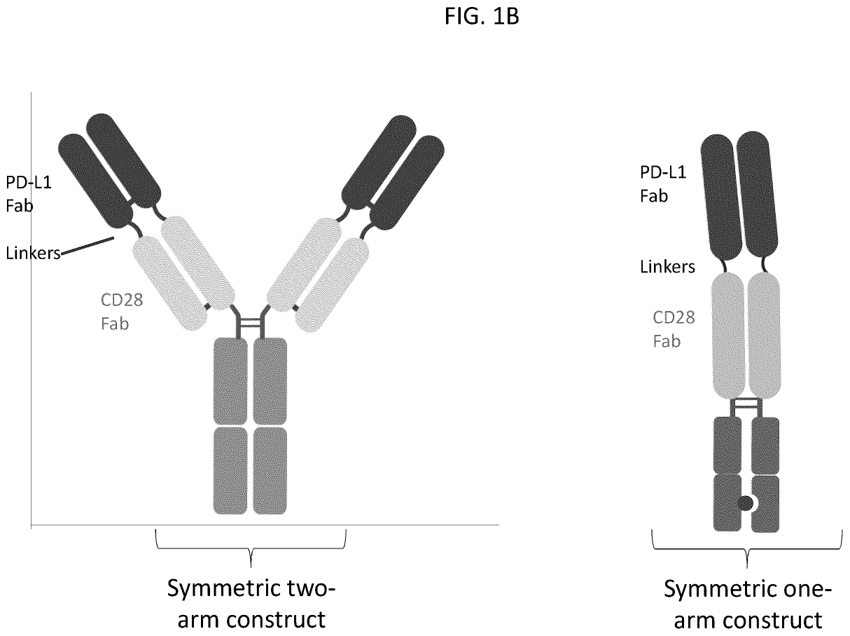

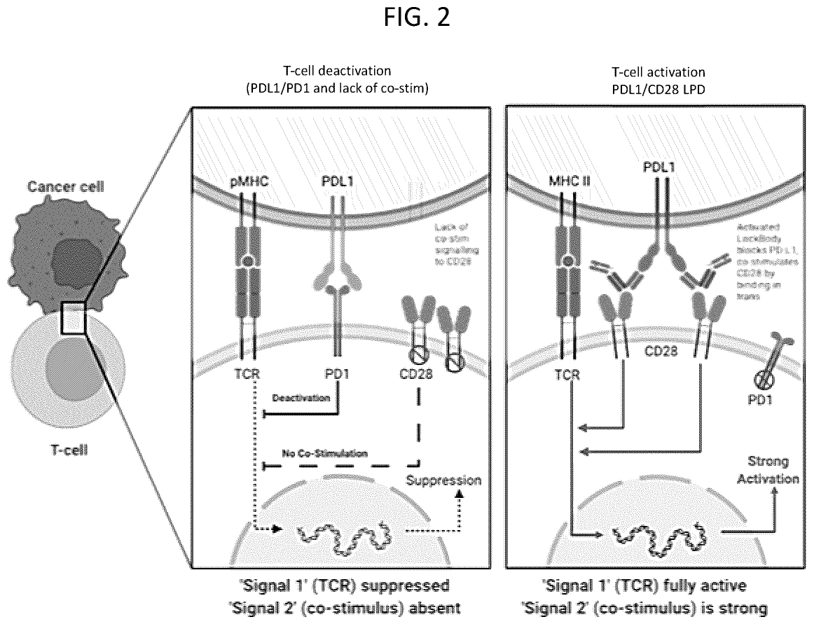

Both factors described above minimize the potential safety and efficacy of bispecific antibodies that drive CD28 engagement and activation. The anti-PD-L1 and anti-CD28 proteins (see, e.g., FIG. 1) provided herein overcome the peripheral sink and toxicity issues by minimizing binding of CD28 outside of diseased tissue. This effect is achieved by adding PD-L1 binding domains and linkers above (i.e., amino-terminal to) the CD28 binding domains. The use of appropriate upper domain and linker combinations results in a configuration that minimizes binding activity in the lower (i.e., carboxy-terminal) CD28 domain. The PD-L1 domain then drives high concentration in PD-L1 enriched tumor microenvironments. The protein construct linker system exploits the elevated MMP and cathepsin activity that is common in solid tumors to cleave the linker peptides, exposing the CD28 binding domains and thereby conditionally activating the CD28-activating activity in the tumor, rather than the periphery. These combined biological functions thereby afford the molecule the potential to avoid the peripheral CD28 sink and maximize T cell immune responses to cancer cells, as outlined in FIG. 2.

Provided herein are proteins that are conditionally active in diseased human tissues. The proteins of the disclosure are fully active in specifically binding and blocking PD-L1 throughout the body; exhibit minimized binding of CD28 in healthy tissue; and become highly activated in CD28 binding and activation once in the PD-L1-positive diseased tissue environment. A protein of the disclosure comprises a CD28 binding domain that is masked by a PD-L1 binding domain in non-diseased tissues. The protein also comprises two peptide linkers that are cleaved by one or more proteases expressed in a diseased tissue (e.g, a tumor). The linker cleavage unmasks the CD28 binding domain in the diseased tissue, thus allowing binding and/or function of the protein selectively in the diseased tissue.

### Protein Molecules

Provided herein are proteins comprising two Fab fragments (an anti-PD-L1 Fab and an anti-CD28 Fab). The protein is monovalent when in the intact structure and can only have a maximum of monovalent CD28 binding when activated, to minimize peripheral toxicity risk associated with bivalent, activating anti-CD28 antibodies.

In some embodiments, a protein comprises a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD28 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD28 VL domain, and a second immunoglobulin light chain constant region. In some embodiments, the heavy chain further comprises an immunoglobulin hinge region and an Fc domain at its C-terminus. In some embodiments, the heavy chain comprises in N-terminus to C-terminus order, the anti-PD-L1 VH domain, the first CH1 domain, the first linker, the anti-CD28 VH domain, the second CH1 domain, a hinge, a CH2 domain, and a CH3 domain.

In some embodiments, the protein further comprises a third polypeptide chain comprising a hinge and a Fc region. The third polypeptide chain may be referred to as a “Fc-stump”.

A diagram of an illustrative protein of the disclosure, with labeled domains, is shown in FIG. 1.

In some embodiments, the linker described herein is cleavable by a protease. In some embodiments, the protease is found in diseased tissues. In some embodiments, the first linker and/or the second linker are cleavable by matrix metalloproteases (MMPs) and/or cathepsins found in diseased tissues, such as tumors. The linkers in the protein are immunoglobulin-derived hinge sequences that are both proteolytically sensitive and may be sequentially cleaved, with a first cleavage taking the intact structure and creating an intermediate active state which allows an anti-PD-L1 Fab and an anti-CD28 Fab from a single protein construct to bind their cognate targets. A second cleavage event in the second linker removes the covalent linkage between the anti-PD-L1 Fab and the anti-CD28 Fab, abrogating the ability of the molecule to recruit T cell killing of PD-L1+ cells. This secondary cleavage event thereby constitutes a “self-destruct mechanism” that minimizes risk of activated molecule escaping the tumor microenvironment. Cleaved linkers based on immunoglobulin hinge sequences may also recruit increased immune effector function (antibody-dependent cellular cytotoxicity (ADCC), complement-dependent cytotoxicity (CDC) and antibody-dependent cellular phagocytosis (ADCP)) at the cell membrane via endogenous anti-hinge antibodies, which are a known phenomenon in human patients with (and even without) underlying autoreactive disease.

In some embodiments, a protein comprises one or more amino acid sequences provided in Table 1 or Table 2.

TABLE 1
Exemplary sequences

| Domain or Region | Sequence | SEQ ID NO |
|---|---|---|
| Linker LHL | GPAPELLGGGS | 1 |
| Linker LHL-F | GPAPLGLGGGS | 2 |
| Linker LHLX | GPAPELLGGGGS | 3 |
| Linker LHLX-F | GPAPLGLGGGGS | 4 |
| Linker LHL-FR | GPAPLGLRGGS | 33 |
| Human CH1 domain | ASTKGPSVFPLAPSSKSTSGGTAALGCLVKDYFPEPVTV | 5 |
| | SWNSGALTSGVHTFPAVLQSSGLYSLSSVVTVPSSSLGT | |
| | QTYICNVNHKPSNTKVDKKVEPKSC | |
| Human CH1-hinge- | ASTKGPSVFPLAPSSKSTSGGTAALGCLVKDYFPEPVTV | 6 |
| CH2—CH3 sequence | SWNSGALTSGVHTFPAVLQSSGLYSLSSVVTVPSSSLGT | |
| (IgG1 wild type) | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLH | |
| | QDWLNGKEYKCKVSNKALPAPIEKTISKAKGQPREPQV | |
| | YTLPPSRDELTKNQVSLTCLVKGFYPSDIAVEWESNGQP | |
| | ENNYKTTPPVLDSDGSFFLYSKLTVDKSRWQQGNVFSC | |
| | SVMHEALHNHYTQKSLSLSPGK | |
| Human Cₖₐₚₚₐ domain | RTVAAPSVFIFPPSDEQLKSGTASVVCLLNNFYPREAKV | 7 |
| (Ck) | QWKVDNALQSGNSQESVTEQDSKDSTYSLSSTLTLSKA | |
| | DYEKHKVYACEVTHQGLSSPVTKSFNRGEC | |
| Human IgG1-3M | ASTKGPSVFPLAPSSKSTSGGTAALGCLVKDYFPEPVTV | 8 |
| | SWNSGALTSGVHTFPAVLQSSGLYSLSSVVTVPSSSLGT | |
| | QTYICNVNHKPSNTKVDKKVEPKSCDKTHTCPPCPAPEA | |
| | AGAPSVFLFPPKPKDTLMISRTPEVTCVVVDVSHEDPEV | |
| | KFNWYVDGVEVHNAKTKPREEQYNSTYRVVSVLTVLH | |
| | QDWLNGKEYKCKVSNKALPAPIEKTISKAKGQPREPQV | |
| | YTLPPSRDELTKNQVSLTCLVKGFYPSDIAVEWESNGQP | |
| | ENNYKTTPPVLDSDGSFFLYSKLTVDKSRWQQGNVFSC | |
| | SVMHEALHNHYTQKSLSLSPGK | |
| Human IgG2 wild | ASTKGPSVFPLAPCSRSTSESTAALGCLVKDYFPEPVTVS | 9 |
| type | WNSGALTSGVHTFPAVLQSSGLYSLSSVVTVPSSNFGTQ | |
| | TYTCNVDHKPSNTKVDKTVERKCCVECPPCPAPPVAGP | |
| | SVFLFPPKPKDTLMISRTPEVTCVVVDVSHEDPEVQFNW | |
| | YVDGVEVHNAKTKPREEQFNSTFRVVSVLTVVHQDWL | |
| | NGKEYKCKVSNKGLPAPIEKTISKTKGQPREPQVYTLPP | |
| | SREEMTKNQVSLTCLVKGFYPSDISVEWESNGQPENNY | |
| | KTTPPMLDSDGSFFLYSKLTVDKSRWQQGNVFSCSVMH | |
| | EALHNHYTQKSLSLSPGK | |
| Human IgG4 wild | ASTKGPSVFPLAPCSRSTSESTAALGCLVKDYFPEPVTVS | 10 |
| type | WNSGALTSGVHTFPAVLQSSGLYSLSSVVTVPSSSLGTK | |
| | TYTCNVDHKPSNTKVDKRVESKYGPPCPSCPAPEFLGGP | |
| | SVFLFPPKPKDTLMISRTPEVTCVVVDVSQEDPEVQFNW | |
| | YVDGVEVHNAKTKPREEQFNSTYRVVSVLTVLHQDWL | |
| | NGKEYKCKVSNKGLPSSIEKTISKAKGQPREPQVYTLPPS | |
| | QEEMTKNQVSLTCLVKGFYPSDIAVEWESNGQPENNYK | |
| | TTPPVLDSDGSFFLYSRLTVDKSRWQEGNVFSCSVMHE | |
| | ALHNHYTQKSLSLSLGK | |
| Human IgG4(S228P) | ASTKGPSVFPLAPCSRSTSESTAALGCLVKDYFPEPVTVS | 11 |
| | WNSGALTSGVHTFPAVLQSSGLYSLSSVVTVPSSSLGTK | |
| | TYTCNVDHKPSNTKVDKRVESKYGPPCPPCPAPEFLGGP | |
| | SVFLFPPKPKDTLMISRTPEVTCVVVDVSQEDPEVQFNW | |
| | YVDGVEVHNAKTKPREEQFNSTYRVVSVLTVLHQDWL | |
| | NGKEYKCKVSNKGLPSSIEKTISKAKGQPREPQVYTLPPS | |
| | QEEMTKNQVSLTCLVKGFYPSDIAVEWESNGQPENNYK | |
| | TTPPVLDSDGSFFLYSRLTVDKSRWQEGNVFSCSVMHE | |
| | ALHNHYTQKSLSLSLGK | |
| Anti-PD-L1 HCDR1 | TYAIS | 12 |
| (Kabat) | | |
| Anti-PD-L1 HCDR2 | GIIPIFGKAHYAQKFQG | 13 |
| (Kabat) | | |
| Anti-PD-L1 HCDR3 | KFHFVSGSPFGMDV | 14 |
| (Kabat) | | |
| Anti-PD-L1 HCDR1 | GDTFSTY | 34 |
| (Chothia) | | |
| Anti-PD-L1 HCDR2 | IPIFGK | 35 |
| (Chothia) | | |
| Anti-PD-L1 HCDR3 | KFHFVSGSPFGMDV | 14 |
| (Chothia) | | |
| Anti-PD-L1 HCDR1 | GDTFSTYA | 36 |
| (IMGT) | | |
| Anti-PD-L1 HCDR2 | IIPIFGKA | 37 |
| (IMGT) | | |
| Anti-PD-L1 HCDR3 | ARKFHFVSGSPFGMDV | 38 |
| (IMGT) | | |
| Anti-PD-L1 LCDR1 | RASQSVSSYLA | 15 |
| (Kabat) | | |
| Anti-PD-L1 LCDR2 | DASNRAT | 16 |
| (Kabat) | | |
| Anti-PD-L1 LCDR3 | QQRSNWPT | 17 |
| (Kabat) | | |
| Anti-PD-L1 LCDR1 | RASQSVSSYLA | 15 |
| (Chothia) | | |
| Anti-PD-L1 LCDR2 | DASNRAT | 16 |
| (Chothia) | | |
| Anti-PD-L1 LCDR3 | QQRSNWPT | 17 |
| (Chothia) | | |
| Anti-PD-L1 LCDR1 | QSVSSY | 39 |
| (IMGT) | | |
| Anti-PD-L1 LCDR2 | DA | |
| (IMGT) | | |
| Anti-PD-L1 LCDR3 | QQRSNWPT | 17 |
| (IMGT) | | |
| Anti-CD28 HCDR1 | SYYIH | 18 |
| Anti-CD28 HCDR2 | SIYPGNVNTNYNEKEKD | 19 |
| Anti-CD28 HCDR3 | SHYGLDWNFDV | 20 |
| Anti-CD28 LCDR1 | QASQNIYVWLN | 21 |
| Anti-CD28 LCDR2 | KASNLHT | 22 |
| Anti-CD28 LCDR3 | QQGQTYPYT | 23 |
| Anti-CD28 HCDR1 | SYYIH | 40 |
| (Kabat) | | |
| Anti-CD28 HCDR2 | SIYPGNVNTNYNEKEKD | 41 |
| (Kabat) | | |
| Anti-CD28 HCDR3 | SHYGLDWNFDV | 42 |
| (Kabat) | | |
| Anti-CD28 HCDR1 | GYTFTSY | 43 |
| (Chothia) | | |
| Anti-CD28 HCDR2 | YPGNVN | 44 |
| (Chothia) | | |
| Anti-CD28 HCDR3 | SHYGLDWNFDV | 42 |
| (Chothia) | | |
| Anti-CD28 HCDR1 | GYTFTSYY | 45 |
| (IMGT) | | |
| Anti-CD28 HCDR2 | IYPGNVNT | 46 |
| (IMGT) | | |
| Anti-CD28 HCDR3 | TRSHYGLDWNFDV | 47 |
| (IMGT) | | |
| Anti-CD28 LCDR1 | QASQNIYVWLN | 48 |
| (Kabat) | | |
| Anti-CD28 LCDR2 | KASNLHT | 49 |
| (Kabat) | | |
| Anti-CD28 LCDR3 | QQGQTYPYT | 50 |
| (Kabat) | | |
| Anti-CD28 LCDR1 | QASQNIYVWLN | 48 |
| (Chothia) | | |
| Anti-CD28 LCDR2 | KASNLHT | 49 |
| (Chothia) | | |
| Anti-CD28 LCDR3 | QQGQTYPYT | 50 |
| (Chothia) | | |
| Anti-CD28 LCDR1 | QNIYVW | 51 |
| (IMGT) | | |
| Anti-CD28 LCDR2 | KA | |
| (IMGT) | | |
| Anti-CD28 LCDR3 | QQGQTYPYT | 50 |
| (IMGT) | | |
| Anti-CD28-2 | GYTFTEYIIH | 53 |
| HCDR1 (Kabat) | | |
| Anti-CD28-2 | IGWFYPGSNDIQYNAQFKG | 54 |
| HCDR2 (Kabat) | | |
| Anti-CD28-2 | RDDFSGYDALPY | 55 |
| HCDR3 (Kabat) | | |
| Anti-CD28-2 | GYTFTEY | 56 |
| HCDR1 (Chothia) | | |
| Anti-CD28-2 | YPGSND | 57 |
| HCDR2 (Chothia) | | |
| Anti-CD28-2 | RDDFSGYDALPY | 55 |
| HCDR3 (Chothia) | | |
| Anti-CD28-2 | GYTFTEYI | 58 |
| HCDR1 (IMGT) | | |
| Anti-CD28-2 | FYPGSNDI | 59 |
| HCDR2 (IMGT) | | |
| Anti-CD28-2 | ARRDDFSGYDALPY | 60 |
| HCDR3 (IMGT) | | |
| Anti-CD28-2 LCDR1 | KTNENIYSNLA | 61 |
| (Kabat) | | |
| Anti-CD28-2 LCDR2 | AATHLVE | 62 |
| (Kabat) | | |
| Anti-CD28-2 LCDR3 | QHFWGTPCT | 63 |
| (Kabat) | | |
| Anti-CD28-2 LCDR1 | KTNENIYSNLA | 61 |
| (Chothia) | | |
| Anti-CD28-2 LCDR2 | AATHLVE | 62 |
| (Chothia) | | |
| Anti-CD28-2 LCDR3 | QHFWGTPCT | 63 |
| (Chothia) | | |
| Anti-CD28-2 LCDR1 | ENIYSN | 64 |
| (IMGT) | | |
| Anti-CD28-2 LCDR2 | AA | |
| (IMGT) | | |
| Anti-CD28-2 LCDR3 | QHFWGTPCT | 63 |
| (IMGT) | | |
| Anti-PD-L1 VH | QVQLVQSGAEVKKPGSSVKVSCKTSGDTFSTYAISWVR | 24 |
| domain | QAPGQGLEWMGGIIPIFGKAHYAQKFQGRVTITADESTS | |
| | TAYMELSSLRSEDTAVYFCARKFHFVSGSPFGMDVWGQ | |
| | GTTVTVSS | |
| Anti-PD-L1 VL | EIVLTQSPATLSLSPGERATLSCRASQSVSSYLAWYQQK | 25 |
| domain | PGQAPRLLIYDASNRATGIPARFSGSGSGTDFTLTISSLEP | |
| | EDFAVYYCQQRSNWPTFGQGTKVEIK | |
| Anti-CD28 VH | QVQLVQSGAEVKKPGASVKVSCKASGYTFTSYYIHWVR | 26 |
| domain | QAPGQGLEWIGSIYPGNVNTNYNEKEKDRATLTVDTSIS | |
| | TAYMELSRLRSDDTAVYFCTRSHYGLDWNFDVWGQGT | |
| | TVTVSS | |
| Anti-CD28 VL | DIQMTQSPSSLSASVGDRVTITCQASQNIYVWLNWYQQ | 27 |
| domain | KPGKAPKLLIYKASNLHTGVPSRFSGSGSGTDFTLTISSL | |
| | QPEDIATYYCQQGQTYPYTFGGGTKVEIK | |
| Anti-CD28-2 VH | QVQLQQSGAELKKPGASVKVSCKASGYTFTEYIIHWIKL | 66 |
| domain | RSGQGLEWIGWFYPGSNDIQYNAQFKGKATLTADKSSS | |
| | TVYMELTGLTPEDSAVYFCARRDDFSGYDALPYWGQG | |
| | TLVTVSA | |
| Anti-CD28-2 VL | DIQMTQSPSSLSASVGDRVTITCKTNENIYSNLAWYQQK | 67 |
| domain | DGKSPQLLIYAATHLVEGVPSRFSGSGSGTQYSLTISSLQ | |
| | PEDFGNYYCQHFWGTPCTFGGGTKLEIK | |

In the VH and VL domain sequences, the CDR sequences are underlined.

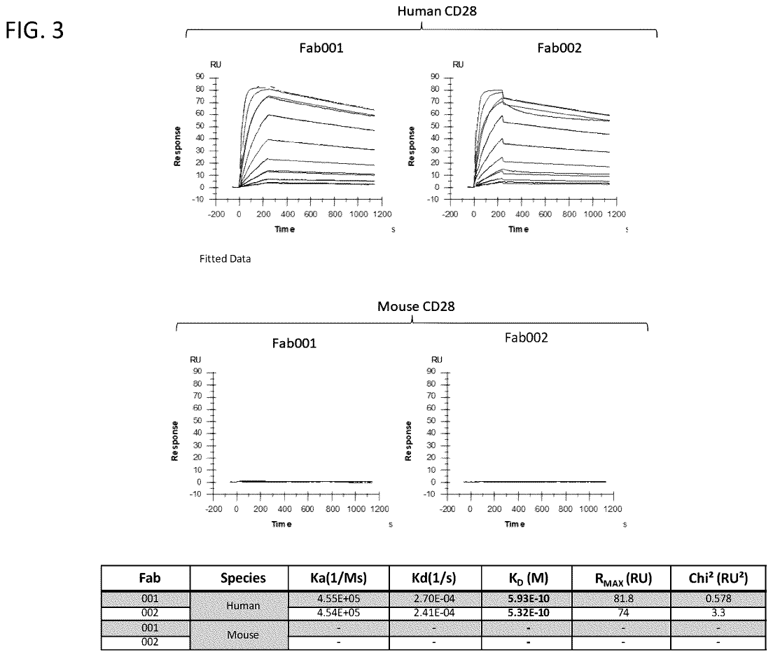

Table 2 describes selected exemplary proteins described herein generated with selected sequences from Table 1 as described. Both anti-CD28 Fab proteins were characterized further in SPR binding experiments as described in FIG. 3.

TABLE 2
Chain sequence combinations of illustrative
anti-CD3 Fab sequences

| Name | Heavy Chain | Light Chain |
|---|---|---|
| Anti-CD28 Fab-001 | Anti - CD28 VH- | Anti-CD28 VL- |
| | Human CH1 domain | Human Cₖₐₚₚₐ |
| | | domain (Ck) |
| Anti-CD3 Fab-002 | Anti - CD28-2 VH - | Anti-CD28 -2 VL- |
| | Human CH1 domain | Human Cₖₐₚₚₐ |
| | | domain (Ck) |

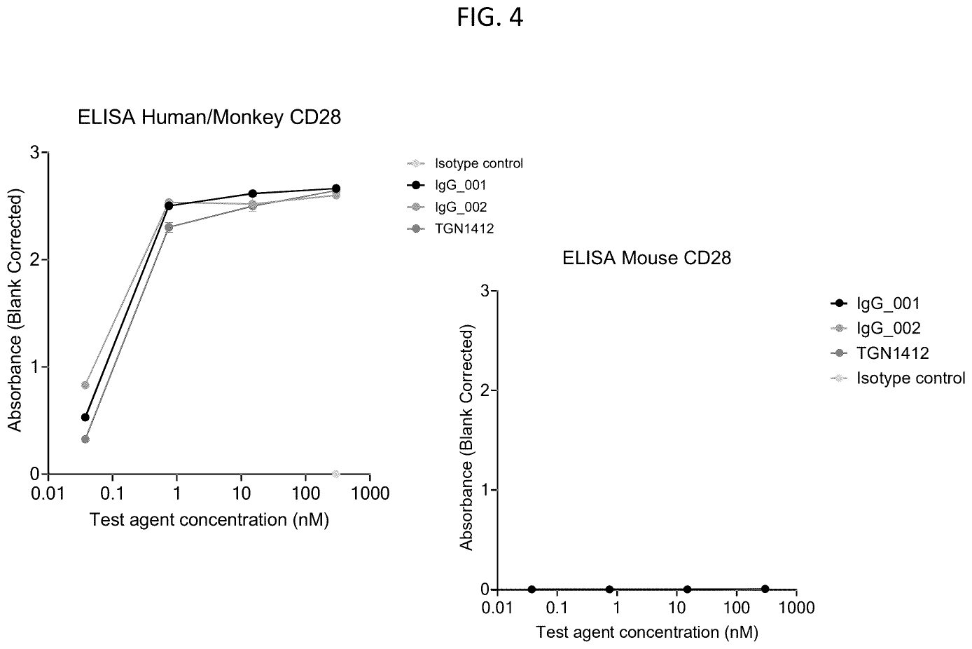

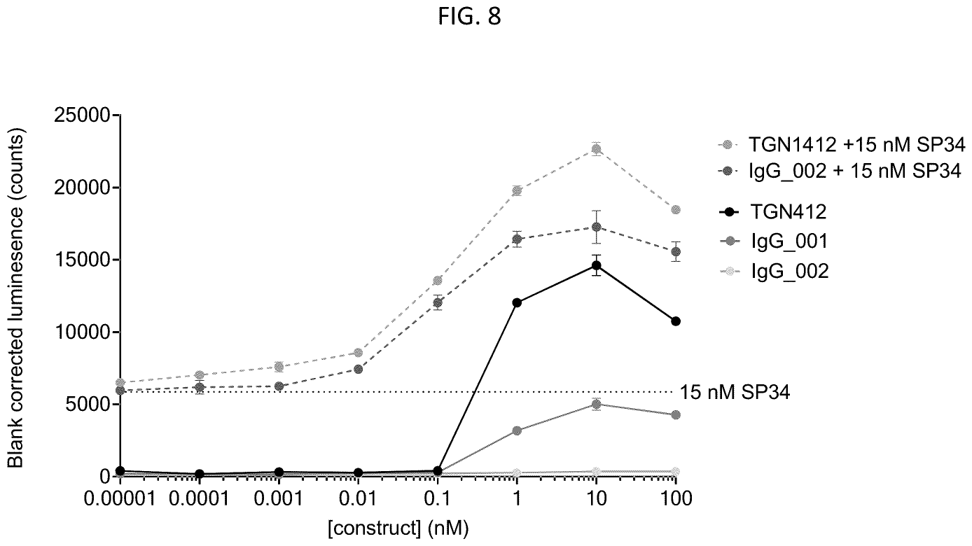

Table 3 describes selected IgG proteins generated with selected sequences from Table 1 as described. Both CD28 IgG proteins were characterized further in ELISA binding experiments and Jurkat reporter assay as described in FIG. 4 and FIG. 8.

TABLE 3
Chain sequence combinations of illustrative
anti-CD28 IgG sequences

| Name | Heavy Chain | Light Chain | Fc |
|---|---|---|---|
| IgG_001 | Anti - CD28 VH- | Anti-CD28 VL- | Human IgG1-3M |
| | Human CH1 domain | Human Cₖₐₚₚₐ | |
| | | domain (Ck) | |
| IgG_002 | Anti - CD28-2 VH - | Anti-CD28 -2 VL- | Human IgG1-3M |
| | Human CH1 domain | Human Cₖₐₚₚₐ | |
| | | domain (Ck) | |

TABLE 4
Full-length sequences of illustrative heavy and light chains

| Name | SEQ ID NO | Amino Acid sequence |
|---|---|---|
| 1L-F | 28 | EIVLTQSPATLSLSPGERATLSCRASQSVSSYLAWYQQKPGQAP |
| | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLGGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC |
| 1L-FX | 29 | EIVLTQSPATLSLSPGERATLSCRASQSVSSYLAWYQQKPGQAP |
| | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLGGGGSDIQMTQSPSSLSASVGDRVTITCQASQNIYVWLNWY |
| | | QQKPGKAPKLLIYKASNLHTGVPSRFSGSGSGTDFTLTISSLQPE |
| | | DIATYYCQQGQTYPYTFGGGTKVEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC |
| 2L-F | 68 | EIVLTQSPATLSLSPGERATLSCRASQSVSSYLAWYQQKPGQAP |
| | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLGGGSDIQMTQSPSSLSASVGDRVTITCKTNENIYSNLAWYQ |
| | | QKDGKSPQLLIYAATHLVEGVPSRFSGSGSGTQYSLTISSLQPE |
| | | DFGNYYCQHFWGTPCTFGGGTKLEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC |
| 1L | 69 | EIVLTQSPATLSLSPGERATLSCRASQSVSSYLAWYQQKPGQAP |
| | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPE |
| | | LLGGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC |
| 1L-FR | 70 | EIVLTQSPATLSLSPGERATLSCRASQSVSSYLAWYQQKPGQAP |
| | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLRGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC |
| 1L-F- | 71 | EIVLTQSPATLSLSPGERATLSCRASQSVSSYLAWYQQKPGQAP |
| G4S_KiH_Fc | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLGGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GECGGGGSGGGGSGGGGSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLSCAVKGFYPSDIAVEWESNGQPENNY |
| | | KTTPPVLDSDGSFFLVSKLTVDKSRWQQGNVFSCSVMHEALH |
| | | NHYTQKSLSLSPGK |
| 1L-FX- | 72 | EIVLTQSPATLSLSPGERATLSCRASQSVSSYLAWYQQKPGQAP |
| G4S_KiH_Fc | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLGGGGSDIQMTQSPSSLSASVGDRVTITCQASQNIYVWLNWY |
| | | QQKPGKAPKLLIYKASNLHTGVPSRFSGSGSGTDFTLTISSLQPE |
| | | DIATYYCQQGQTYPYTFGGGTKVEIKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSFN |
| | | RGECGGGGSGGGGSGGGGSVFLFPPKPKDTLMISRTPEVTCVV |
| | | VDVSHEDPEVKFNWYVDGVEVHNAKTKPREEQYNSTYRVVS |
| | | VLTVLHQDWLNGKEYKCKVSNKALPAPIEKTISKAKGQPREPQ |
| | | VYTLPPSRDELTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| 1L- | 73 | EIVLTQSPATLSLSPGERATLSCRASQSVSSYLAWYQQKPGQAP |
| G4S_KiH_Fc | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPE |
| | | LLGGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GECGGGGSGGGGSGGGGSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLSCAVKGFYPSDIAVEWESNGQPENNY |
| | | KTTPPVLDSDGSFFLVSKLTVDKSRWQQGNVFSCSVMHEALH |
| | | NHYTQKSLSLSPGK |
| 1L-FR- | 74 | EIVLTQSPATLSLSPGERATLSCRASQSVSSYLAWYQQKPGQAP |
| G4S_KiH_Fc | | RLLIYDASNRATGIPARFSGSGSGTDFTLTISSLEPEDFAVYYCQQ |
| | | RSNWPTFGQGTKVEIKRTVAAPSVFIFPPSDEQLKSGTASVVC |
| | | LLNNFYPREAKVQWKVDNALQSGNSQESVTEQDSKDSTYSLS |
| | | STLTLSKADYEKHKVYACEVTHQGLSSPVTKSFNRGECGPAPL |
| | | GLRGGSDIQMTQSPSSLSASVGDRVTITCQASQNIYVWLNWYQ |
| | | QKPGKAPKLLIYKASNLHTGVPSRFSGSGSGTDFTLTISSLQPED |
| | | IATYYCQQGQTYPYTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSFNR |
| | | GECGGGGSGGGGSGGGGSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSRDELTKNQVSLSCAVKGFYPSDIAVEWESNGQPENNY |
| | | KTTPPVLDSDGSFFLVSKLTVDKSRWQQGNVFSCSVMHEALH |
| | | NHYTQKSLSLSPGK |
| 1H- | 30 | QVQLVQSGAEVKKPGSSVKVSCKTSGDTFSTYAISWVRQAPG |
| KiH_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPCRDELTK |
| | | NQVSLWCLVKGFYPSDIAVEWESNGQPENNYKTTPPVLDSDG |
| | | SFFLYSKLTVDKSRWQQGNVFSCSVMHEALHNHYTQKSLSLS |
| | | PGK |
| 1HX- | 31 | QVQLVQSGAEVKKPGSSVKVSCKTSGDTFSTYAISWVRQAPG |
| KiH_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGGSQVQLVQSGAEVKKPGASVK |
| | | VSCKASGYTFTSYYIHWVRQAPGQGLEWIGSIYPGNVNTNYNE |
| | | KEKDRATLTVDTSISTAYMELSRLRSDDTAVYFCTRSHYGLDW |
| | | NFDVWGQGTTVTVSSASTKGPSVFPLAPSSKSTSGGTAALGCL |
| | | VKDYFPEPVTVSWNSGALTSGVHTFPAVLQSSGLYSLSSVVTV |
| | | PSSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPCRDELTK |
| | | NQVSLWCLVKGFYPSDIAVEWESNGQPENNYKTTPPVLDSDG |
| | | SFFLYSKLTVDKSRWQQGNVFSCSVMHEALHNHYTQKSLSLS |
| | | PGK |
| 2H_ | 75 | QVQLVQSGAEVKKPGSSVKVSCKTSGDTFSTYAISWVRQAPG |
| Ki_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGSQVQLQQSGAELKKPGASVKV |
| | | SCKASGYTFTEYIIHWIKLRSGQGLEWIGWFYPGSNDIQYNAQF |
| | | KGKATLTADKSSSTVYMELTGLTPEDSAVYFCARRDDFSGYD |
| | | ALPYWGQGTLVTVSAASTKGPSVFPLAPSSKSTSGGTAALGCL |
| | | VKDYFPEPVTVSWNSGALTSGVHTFPAVLQSSGLYSLSSVVTV |
| | | PSSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPCRDELTK |
| | | NQVSLWCLVKGFYPSDIAVEWESNGQPENNYKTTPPVLDSDG |
| | | SFFLYSKLTVDKSRWQQGNVFSCSVMHEALHNHYTQKSLSLS |
| | | PGK |
| 1H- | 76 | QVQLVQSGAEVKKPGSSVKVSCKTSGDTFSTYAISWVRQAPG |
| FR_KiH_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPLGLRGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPCRDELTK |
| | | NQVSLWCLVKGFYPSDIAVEWESNGQPENNYKTTPPVLDSDG |
| | | SFFLYSKLTVDKSRWQQGNVFSCSVMHEALHNHYTQKSLSLS |
| | | PGK |
| 1H- | 77 | QVQLVQSGAEVKKPGSSVKVSCKTSGDTFSTYAISWVRQAPG |
| G4S_KiH_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCGGGGSGGGGSG |
| | | GGGSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSRDELTKNQV |
| | | SLWCLVKGFYPSDIAVEWESNGQPENNYKTTPPVLDSDGSFFL |
| | | YSKLTVDKSRWQQGNVFSCSVMHEALHNHYTQKSLSLSPGK |
| 1HX- | 78 | QVQLVQSGAEVKKPGSSVKVSCKTSGDTFSTYAISWVRQAPG |
| G4S_KiH_Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGGSQVQLVQSGAEVKKPGASVK |
| | | VSCKASGYTFTSYYIHWVRQAPGQGLEWIGSIYPGNVNTNYNE |
| | | KEKDRATLTVDTSISTAYMELSRLRSDDTAVYFCTRSHYGLDW |
| | | NFDVWGQGTTVTVSSASTKGPSVFPLAPSSKSTSGGTAALGCL |
| | | VKDYFPEPVTVSWNSGALTSGVHTFPAVLQSSGLYSLSSVVTV |
| | | PSSSLGTQTYICNVNHKPSNTKVDKKVEPKSCGGGGSGGGGSG |
| | | GGGSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSRDELTKNQV |
| | | SLWCLVKGFYPSDIAVEWESNGQPENNYKTTPPVLDSDGSFFL |
| | | YSKLTVDKSRWQQGNVFSCSVMHEALHNHYTQKSLSLSPGK |
| 1H- | 79 | QVQLVQSGAEVKKPGSSVKVSCKTSGDTFSTYAISWVRQAPG |
| FR-G4S_ | | QGLEWMGGIIPIFGKAHYAQKFQGRVTITADESTSTAYMELSSL |
| KIH_Fc | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPLGLRGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCGGGGSGGGGSG |
| | | GGGSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSRDELTKNQV |
| | | SLWCLVKGFYPSDIAVEWESNGQPENNYKTTPPVLDSDGSFFL |
| | | YSKLTVDKSRWQQGNVFSCSVMHEALHNHYTQKSLSLSPGK |
| 1H-Fc | 80 | QVQLVQSGAEVKKPGSSVKVSCKTSGDTFSTYAISWVRQAPG |
| | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDELTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSP |
| | | GK |
| 2H-Fc | 81 | QVQLVQSGAEVKKPGSSVKVSCKTSGDTFSTYAISWVRQAPG |
| | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPELLGGGSQVQLQQSGAELKKPGASVKV |
| | | SCKASGYTFTEYIIHWIKLRSGQGLEWIGWFYPGSNDIQYNAQF |
| | | KGKATLTADKSSSTVYMELTGLTPEDSAVYFCARRDDFSGYD |
| | | ALPYWGQGTLVTVSAASTKGPSVFPLAPSSKSTSGGTAALGCL |
| | | VKDYFPEPVTVSWNSGALTSGVHTFPAVLQSSGLYSLSSVVTV |
| | | PSSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDELTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSP |
| | | GK |
| 1H- | 82 | QVQLVQSGAEVKKPGSSVKVSCKTSGDTFSTYAISWVRQAPG |
| FR-Fc | | QGLEWMGGIIPIFGKAHYAQKFQGRVTITADESTSTAYMELSSL |
| | | RSEDTAVYFCARKFHFVSGSPFGMDVWGQGTTVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKKVEPKSCGPAPLGLRGGSQVQLVQSGAEVKKPGASVKV |
| | | SCKASGYTFTSYYIHWVRQAPGQGLEWIGSIYPGNVNTNYNEK |
| | | EKDRATLTVDTSISTAYMELSRLRSDDTAVYFCTRSHYGLDWN |
| | | FDVWGQGTTVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCDKTHTCPPCPAP |
| | | EAAGAPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDELTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSP |
| | | GK |
| G4S- | 83 | GGGGSGGGGSGGGGSVFLFPPKPKDTLMISRTPEVTCVVVDVS |
| Holes- | | HEDPEVKFNWYVDGVEVHNAKTKPREEQYNSTYRVVSVLTV |
| Fc | | LHQDWLNGKEYKCKVSNKALPAPIEKTISKAKGQPREPQVYTL |
| | | PPSRDELTKNQVSLSCAVKGFYPSDIAVEWESNGQPENNYKTT |
| | | PPVLDSDGSFFLVSKLTVDKSRWQQGNVFSCSVMHEALHNHY |
| | | TQKSLSLSPGK |
| G4S- | 84 | GGGGSGGGGSGGGGSVFLFPPKPKDTLMISRTPEVTCVVVDVS |
| Knobs- | | HEDPEVKFNWYVDGVEVHNAKTKPREEQYNSTYRVVSVLTV |
| Fc | | LHQDWLNGKEYKCKVSNKALPAPIEKTISKAKGQPREPQVYTL |
| | | PPSRDELTKNQVSLWCLVKGFYPSDIAVEWESNGQPENNYKTT |
| | | PPVLDSDGSFFLYSKLTVDKSRWQQGNVFSCSVMHEALHNHY |
| | | TQKSLSLSPGK |
| Fc- | 32 | SCPPCPAPEAAGAPSVFLFPPKPKDTLMISRTPEVTCVVVDVSH |
| stump | | EDPEVKFNWYVDGVEVHNAKTKPREEQYNSTYRVVSVLTVL |
| | | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYT |
| | | QKSLSLSPGK |

The anti-PD-L1/anti-CD28 protein design may be based on sequences derived from IgG1, IgG2, IgG3, IgG4, IgE, IgM, or IgA and may or may not have effector function capacity.

Proteins disclosed herein comprise domains and regions of antibody molecules. The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. IgG, IgA, and IgD constant regions comprise a flexible hinge region between the CH1 domain and the CH2 domain. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4. The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, Chothia, C. et al. (1987) J. Mol. Biol. 196:901-917, or IMGT Lefranc MP. Unique database numbering system for immunogenetic analysis. Immunol Today. 1997 November; 18 (11): 509.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

A protein provided herein comprises two Fab fragments. A Fab fragment is a monovalent antigen-binding fragment consisting of the VL, VH, CL and CH1 domains.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies. As used herein, the terms “immunological binding” and “immunological binding properties” refer to the non-covalent interactions of the type which occur between an immunoglobulin molecule (e.g., antibody or antigen-binding portion thereof), or a protein comprising an immunoglobulin-derived binding domain(s) and an antigen for which the immunoglobulin or protein is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind PD-L1 or CD28 when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

Functionally, the binding affinity of a protein provided herein may be within the range of 10⁻⁵ M to 10⁻¹² M. For example, the binding affinity of a protein provided herein is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD28 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD28 VL domain, and a second immunoglobulin light chain constant region.

In some embodiments, the heavy chain comprises in N-terminus to C-terminus order, the anti-PD-L1 VH domain, the first CH1 domain, the first linker, the anti-CD28 VH domain, the second CH1 domain, a hinge, a CH2 domain, and a CH3 domain.

In some embodiments, the protein further comprises a third polypeptide chain comprising a hinge and a Fc region. In some embodiments, the third polypeptide chain comprises the amino acid sequence of SEQ ID NO: 32.

In some embodiments, the first linker comprises the amino acid sequence of SEQ ID NO: 1, SEQ ID NO: 2, SEQ ID NO: 3, or SEQ ID NO: 4. In some embodiments, the second linker comprises the amino acid sequence of SEQ ID NO: 1, SEQ ID NO: 2, SEQ ID NO: 3, or SEQ ID NO: 4.

In some embodiments, the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14; the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17; the anti-CD28 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and the anti-CD28 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

In some embodiments, the anti-PD-L1 VH domain comprises the amino acid sequence of SEQ ID NO: 24, and the anti-PD-L1 VL domain comprises the amino acid sequence of SEQ ID NO: 25.

In some embodiments, the anti-CD28 VH domain comprises the amino acid sequence of SEQ ID NO: 26.

In some embodiments, the anti-CD28 VL domain comprises the amino acid sequence of SEQ ID NO: 27.

In some embodiments, the heavy chain comprises the amino acid sequence of SEQ ID NO: 30 or SEQ ID NO: 31. In some embodiments, the heavy chain comprises an amino acid sequence that is at least 90%, 91%, 92%, 93%, 94%, 95%, 96%, 97%, 98% or 99% identical to the amino acid sequence of SEQ ID NO: 30 or SEQ ID NO: 31.

In some embodiments, the light chain comprises the amino acid sequence of SEQ ID NO: 28 or SEQ ID NO: 29. In some embodiments, the heavy chain comprises an amino acid sequence that is at least 90%, 91%, 92%, 93%, 94%, 95%, 96%, 97%, 98% or 99% identical to the amino acid sequence of SEQ ID NO: 28 or SEQ ID NO: 29.

In some embodiments, provided herein is a protein wherein
(a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
(b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.

Also provided herein is a protein comprising a heavy chain and a light chain, wherein the heavy chain comprises an amino acid sequence provided herein, with 1, 2 or 3 conservative amino acid substitutions; and wherein the light chain comprises an amino acid sequence provided herein, with 1, 2 or 3 conservative amino acid substitutions. In some embodiments, conservative amino acid substitutions are made only in the FR sequences and not in the CDR sequences. In some embodiments, conservative amino acid substitutions are not made in the first linker or the second linker sequences.

In some embodiments, a protein provided herein comprises an immunoglobulin heavy chain constant region at the C-terminus of the heavy chain. In some embodiments, the immunoglobulin heavy chain constant region is IgG, IgE, IgM, IgD, IgA or IgY. In some embodiments, the immunoglobulin heavy chain constant region is IgG1, IgG2, IgG3, IgG4, IgAQ1 or IgA2. In some embodiments, the immunoglobulin heavy chain constant region is IgG1. In some embodiments, the immunoglobulin heavy chain constant region is immunologically inert. In some embodiments, the immunoglobulin heavy chain constant region comprises one or more mutations to reduce or prevent FcγR binding, antibody-dependent cell-mediated cytotoxicity (ADCC) activity, antibody-dependent cellular phagocytosis (ADCP), and/or complement-dependent cytotoxicity (CDC) activity. In some embodiments, the immunoglobulin heavy chain constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments, a protein provided herein may comprise an immunoglobulin light chain constant region that is a kappa light chain. In some embodiments a kappa light chain comprises SEQ ID NO: 7.

In some embodiments, a protein provided herein may comprise an immunoglobulin light chain constant region that is a lambda light chain.

In some embodiments, a protein provided herein may comprise an immunoglobulin heavy chain constant region comprising an amino acid sequence of an Fc region of human IgG4, human IgG4 (S228P), human IgG2, human IgG1, human IgG1 effector null. For example, the human IgG4 (S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, a protein may comprise an immunoglobulin heavy chain constant region comprising the amino acid sequence of any one of SEQ ID NO: 5, SEQ ID NO: 6, SEQ ID NO: 8, SEQ ID NO: 9, SEQ ID NO: 10 and SEQ ID NO: 11.

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example, RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers. Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

TABLE 5
Chain sequence combinations used in illustrative proteins

| | Heavy Chain | Light Chain | Heavy Chain |
|---|---|---|---|
| Name | Construct | Construct | Construct 2 |
| LPD28-1 | 1H-KiH_Fc | 1L | Fc-stump |
| | (SEQ ID NO: 30) | (SEQ ID NO: 69) | (SEQ ID NO: 32) |
| LPD28-2 | 1HX-KiH-Fc | 1LX | Fc-stump |
| | (SEQ ID NO: 31) | (SEQ ID NO: 29) | (SEQ ID NO: 32) |
| LPD28-3 | 2H-KiH_Fc | 2L-F | Fc-stump |
| | (SEQ ID NO: 75) | (SEQ ID NO: 68) | (SEQ ID NO: 32) |
| LPD28-4 | 1H-FR-KiH_Fc | 1L | Fc-stump |
| | (SEQ ID NO: 76) | (SEQ ID NO: 69) | (SEQ ID NO: 32) |
| LPD28-5 | 1H-KiH_Fc | 1L-FR | Fc-stump |
| | (SEQ ID NO: 30) | (SEQ ID NO: 70) | (SEQ ID NO: 32) |
| LPD28-6 | 1H-G4S_KiH_Fc | 1L-F-G4S_KiH_Fc | |
| | (SEQ ID NO: 77) | (SEQ ID NO: 71) | |
| LPD28-7 | 1HX-G4S_KiH_Fc | 1L-FX-G4S_KiH_Fc | |
| | (SEQ ID NO: 78) | (SEQ ID NO: 72) | |
| LPD28-8 | 1H-FR-G4S_KiH_Fc | 1L-G4S_KiH_Fc | |
| | (SEQ ID NO: 79) | (SEQ ID NO: 73) | |
| LPD28-9 | 1H-G4S_KiH_Fc | 1L-FR-G4S_KiH_Fc | |
| | (SEQ ID NO: 77) | (SEQ ID NO: 74) | |
| LPD28-10 | 1H-Fc | 1L-F | |
| | (SEQ ID NO: 80) | (SEQ ID NO: 28) | |
| LPD28-11 | 2H-Fc | 2L-F | |
| | (SEQ ID NO: 81) | (SEQ ID NO: 68) | |
| LPD28-12 | 1H-FR-Fc | 1L | |
| | (SEQ ID NO: 82) | (SEQ ID NO: 69) | |
| LPD28-13 | 1H-Fc | 1L-FR | |
| | (SEQ ID NO: 80) | (SEQ ID NO: 70) | |

### Pharmaceutical Compositions

The activatable proteins provided herein (also referred to herein as “active compounds”) can be incorporated into pharmaceutical compositions suitable for administration. Such compositions typically comprise a protein (or an immunoconjugate comprising said protein), and a pharmaceutically acceptable carrier, diluent or excipient. Such materials should be non-toxic and should not interfere with the efficacy of the protein. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the protein, an increase in its lifespan and/or in its efficacy in the body or an increase in its solubility in solution.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, NJ) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the protein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Proteins

Provided herein is a nucleic acid molecule (e.g., an isolated nucleic acid molecule) encoding an amino acid sequence of a protein disclosed herein (or an amino acid sequence of a (i) VH domain, (ii) a VL domain, or (iii) both a VH domain and a VL domain of a protein). Further provided herein is a nucleic acid molecule (e.g., an isolated nucleic acid molecule) encoding (i) a heavy chain, (ii) a light chain, or (iii) both a heavy chain and a light chain of a protein disclosed herein. In some embodiments, a nucleic acid molecule encoding a VH domain, a VL domain, a heavy chain or a light chain comprises a signal sequence (or encodes a leader peptide). In some embodiments, a nucleic acid molecule encoding a VH domain, a VL domain, a heavy chain or a light chain does not comprise a signal sequence (or does not encode a leader peptide).

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a heavy chain and a light chain of a protein disclosed herein. In some embodiments, a recombinant host cell comprises (i) a first vector or a first nucleic acid molecule encoding a heavy chain of a protein disclosed herein and (ii) a second vector or a second nucleic acid molecule encoding a light chain of a protein disclosed herein.

Protein molecules of the invention, or portions thereof, can be produced using techniques well known in the art, for example, recombinant technologies, phage display technologies, synthetic technologies, computational technologies or combinations of such technologies or other technologies readily known in the art.

Further provided herein is a method for producing a protein disclosed herein, the method comprising: culturing a recombinant host cell comprising an expression vector described herein under conditions whereby the nucleic acid segment is expressed, thereby producing the protein. The protein may then be isolated from the host cell or culture. Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Proteins disclosed herein can be produced by any of a variety of methods known to those skilled in the art. In certain embodiments, proteins disclosed herein can be produced recombinantly. For example, nucleic acid sequences encoding one or more of the heavy chains or light chains provided herein, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, light chain proteins and heavy chain proteins are produced in a cell with a signal sequence that is removed upon production of a mature protein disclosed herein.

Those skilled in the art will be able to determine whether a protein comprising a given polypeptide sequence binds to PD-L1 protein and/or CD28 protein using standard methodologies, for example, Western blots, ELISA, and the like.

### Medical Uses of Activatable Proteins

Provided herein are methods and uses of activatable proteins, immunoconjugates, and pharmaceutical compositions disclosed herein for providing a therapeutic benefit to a subject with cancer.

An activatable protein, immunoconjugate, or pharmaceutical composition disclosed herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the protein, immunoconjugate, or pharmaceutical composition to a subject in need thereof.

Provided herein is a method for enhancing an anti-cancer immune response in a subject, the method comprising administering to the subject a therapeutically effective amount of a protein, an immunoconjugate, or a pharmaceutical composition disclosed herein. In some embodiments, an anti-cancer immune response is a T cell response. In some embodiments, an anti-cancer immune response is a complement response.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, immunoconjugate, or a pharmaceutical composition disclosed herein.

Provided herein is a method for reducing the size of a tumor in a subject, the method comprising administering to the subject a therapeutically effective amount of a protein, an immunoconjugate, or a pharmaceutical composition disclosed herein.

Provided herein is a method for inhibiting the growth of a tumor in a subject, the method comprising administering to the subject a therapeutically effective amount of a protein, an immunoconjugate, or a pharmaceutical composition disclosed herein.

In some embodiments, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

In some embodiments, the cancer is a solid tumor. In some embodiments, the cancer is a hematological cancer.

In some embodiments, a cancer of hematological tissues is a lymphoma. In some embodiments, the cancer is mantle cell lymphoma, acute lymphoblastic leukemia, chronic lymphocytic leukemia, Non-Hodgkin's lymphoma, Hodgkin's lymphoma, acute myeloid leukemia (AML), B-lymphoid leukemia, blastic plasmocytoid dendritic neoplasm (BPDCN), or hairy cell leukemia.

As used herein, the term “effective amount” or “therapeutically effective amount” refers to the amount of a pharmaceutical agent, e.g., a protein, an immunoconjugate, or a pharmaceutical composition disclosed herein, which is sufficient to reduce or ameliorate the severity and/or duration of a cancer, or one or more symptoms thereof, prevent the advancement of a disease, cause regression of a disease, prevent the recurrence, development, onset or progression of one or more symptoms associated with a disease, or enhance or improve the prophylactic or therapeutic effect(s) of another related therapy (e.g., prophylactic or therapeutic agent) for a cancer.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g., decisions on dosage etc., is within the responsibility of general practitioners and other medical doctors and may depend on the severity of the symptoms and/or progression of a disease being treated. Appropriate doses of antibody-based protein molecules are well known in the art (Ledermann J. A. et al., 1991, Int. J. Cancer 47:659-664; Bagshawe K. D. et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody-based protein molecule may be determined by comparing its in vitro activity and in vivo activity in an animal model. Methods for extrapolation of effective dosages in mice and other test animals to humans are known. The precise dose will depend upon a number of factors, including whether the antibody-based protein is for prevention or for treatment, the size and location of the area to be treated, the precise nature of the antibody-based protein, and the nature of any detectable label or other molecule attached to the antibody-based protein.

A typical protein dose will be in the range 100 μg to 1 g for systemic applications, and 1 μg to 1 mg for intradermal injection. An initial higher loading dose, followed by one or more lower doses, may be administered. In some embodiments, the protein is an IgG1 or IgG4 isotype. A dose for a single treatment of an adult subject may be proportionally adjusted for children and infants. Treatments may be repeated at daily, twice-weekly, weekly or monthly intervals, at the discretion of the physician. The treatment schedule for a subject may be dependent on the pharmacokinetic and pharmacodynamic properties of the protein composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, a protein, an immunoconjugate, or a pharmaceutical composition disclosed herein may be administered as a sub-cutaneous injection. Sub-cutaneous injections may be administered using an auto-injector, for example for long term prophylaxis/treatment.

In some embodiments, the therapeutic effect of a protein, an immunoconjugate, or a pharmaceutical composition disclosed herein may persist for several half-lives, depending on the dose. For example, the therapeutic effect of a single dose of a protein, an immunoconjugate, or a pharmaceutical composition disclosed herein may persist in a subject for 1 month or more, 2 months or more, 3 months or more, 4 months or more, 5 months or more, or 6 months or more.

In some embodiments, a subject may be treated with a protein, an immunoconjugate, or a pharmaceutical composition disclosed herein and an additional therapeutic agent or therapy that is used to treat a cancer or a symptom or complication of a cancer. The protein, immunoconjugate, or pharmaceutical composition disclosed herein and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is a protein, an immunoconjugate, or a pharmaceutical composition disclosed herein, for use in the treatment of a disease or a disorder.

Provided herein is a protein, an immunoconjugate, or a pharmaceutical composition disclosed herein, for use as a medicament.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

The terms “amino-terminal”, “N-terminus”, “carboxyl-terminal”, and “C-terminus” are used herein to denote positions within polypeptide chains. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence but is not necessarily at the carboxyl-terminus of the complete polypeptide.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | −3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The term “immunoconjugate” refer to a protein of the disclosure that is conjugated to a cytotoxic, a cytostatic and/or a therapeutic agent.

The term “isolated molecule” (where the molecule is, for example, a protein, a nucleic acid, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “inhibit”, “block”, or “neutralize”, as used herein with respect to bioactivity of a protein disclosed herein means the ability of the protein to substantially antagonize, prohibit, prevent, restrain, slow, disrupt, eliminate, stop, reduce or reverse for example progression, strength, or severity of that which is being inhibited including, but not limited to, the binding of PD-L1 to PD-1, or the binding of CD28 to CD80 and/or CD86.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

PD-L1 is also known as programmed cell death ligand 1, CD274, B7-H, B7H1, PDCD1L1, PDCD1LG1, PDL1, and hPD-L1.

CD28 is also known as cluster of differentiation 28 and TP44. CD28 is a type I transmembrane protein.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a protein or a pharmaceutical composition provided herein that is effective to treat or prevent a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

As used herein and unless otherwise stated, the terms “hinge linker”, “linker”, “hinge”, “first linker”, “second linker”, “lower hinge linker” (“LHL”), “inter-Fab linker”, and derivations thereof, in plural or singular form, refer to a sequence, for example derived from an immunoglobulin hinge region, that can link two polypeptides, for example polypeptides of different Fab regions, and is separate from any hinge sequence in an immunoglobulin hinge region that may be part of a protein of the present invention.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

The disclosure will be further clarified by the following example, which is intended to be purely exemplary of the disclosure and in no way limiting.

### EXAMPLE

The present disclosure will be more specifically illustrated by the following Examples. However, it should be understood that the present disclosure is not limited by these examples in any manner.

The examples of the present disclosure describe the generation and optimization of the conditionally active therapeutic proteins disclosed herein.

### Example 1: Materials and Methods

### Cloning, Transient Expression, Purification and Characterization of Proteins

Polypeptide-encoding DNA sequences were cloned via restriction-ligation cloning into separate human IgG1 heavy and light-chain constant region-encoding expression cassettes in separate plasmid vectors, to create activatable constructs for expression. Proteins were expressed in CHO cells and purified from culture supernatants via a combination of Protein A affinity chromatography (ProA), Ion Exchange Chromatography (IEX) and/or Size Exclusion Chromatography (SEC). Purified proteins were characterized by SEC, SDS-PAGE and mass spectrometry.

### Metalloprotease Digestion

Protein constructs were incubated for time increments between 0 and 24 hours at 37° C. with either full lengths human Matrix Metalloprotease (MMP) enzyme (LPD28-1 and LPD28-2) or catalytic MMP12 domain only (LPD28-3, -4, -5, -6, 7, -8, -9, -10, -11, -12, -13). MMP12 enzyme as added at a ratio of 1% total MMP to protein construct (wt/wt) in Tris buffered saline (pH7.4) containing 5 mM CaCl₂). The reactions were stopped by the addition of 20 mM EDTA and then samples tested for binding or functional activity as described.

### Surface Plasmon Resonance of Fab Proteins

In order to assess the binding of test Fabs to human or mouse CD28 (Acro Biosystems, Newark, USA), multi-cycle kinetic analysis was performed at 25° C. on a Biacore® 8K (serial no. 2724204) HBS-P+ (Cytiva, Marlborough, USA) supplemented with 0.1% BSA (Sigma, Dorset, UK) was used as running buffer as well as for ligand and analyte dilutions. CD28 proteins were diluted to 1.0 μg/mL in running buffer and at the start of each cycle loaded onto F_{c}2, of a series S CM5 chip (Cytiva, Marlborough, USA) previously coupled with an anti-His capture antibody (Cytiva, Marlborough, USA) using standard amine chemistry. Ligand was captured at a flow rate of 10 μl/min to give an immobilization level (R_{L}) of ~60 RU. The surface was then allowed to stabilize. Multi-cycle kinetic data was obtained antigens as the analyte injected at a flow rate of 30 μl/min to try and minimize any potential mass transfer effects. A 9 point 2-fold dilution of purified Fab titrated from 0.39 nM to 100 nM was prepared in running buffer for Fab proteins. For each concentration, the association phases were monitored for 240 seconds, and the dissociation phase was measured for 900 seconds. Regeneration of the sensor chip surface was conducted between cycles using 10 mM glycine pH 2.1. Multiple repeats of a blank and of antigen were programmed into the kinetic run, in order to check the stability of both the surface and analyte over the kinetic cycles. The signal from the reference F_{c}1 (no ligand captured) was subtracted from that of F_{c}2 to correct for bulk effect and differences in non-specific binding to a reference surface. The signal from each blank run (ligand captured but no antigen) was subtracted to correct for differences in surface stability. Binding was analysed using 1:1 binding analysis due to the high affinity interaction between the antibody and antigen. The data was analysed using a procedure referred to as double referencing. This refers to the process of first subtracting the reference channel response (F_{c}1) and then subtracting the zero concentration sensorgram to compensate for bulk effects, baseline drift and small differences between the reference and the active channel.

### T-Cell Activation Bioassay

Functional activity of protein constructs was assessed in a co-culture assay using MDA-MB-231 (PD-L1 high) with a Jurkat reporter cell line (Promega-IL2 Promotor). MDA-MB-231 (PD-L1 high) cells (500000 cell/well for 96-well or 50000 cell/well for 384-well) were seeded into 96-well or 384-well white clear bottomed tissue culture treated plates in Hybri-Care medium (ATCC) supplemented with 10% FBS and incubated at 37° C. overnight in a CO₂ incubator. Medium was removed and control antibodies or protein constructs (+/− pre-digestion with MMP3/7/12) prepared in assay medium (RPMI supplemented with 10% FBS) were added to the cells. Jurkat Effector cells (IL2) were thawed and diluted according to the manufacturer's protocol, then added to the assay wells. Following a 6-hour incubation at 37° C. in a CO₂ incubator the plates were re-equilibrated to room temperature and luciferase activity determined by addition of Bio-Glo reagent for 5-10 minutes and measurement of luminescent signal (RLU). Fold induction was determined by calculating the ratio of the sample RLU/RLU in the absence of antibody following subtraction of background luminescence signal.

### ELISA Binding of IgG and LB Proteins

For ELISA binding assays, 384 well or 96 well plates were coated with 1 ug/ml and incubated overnight at 4° C., protected from light. Plates were washed twice with PBS/0.05% Tween 20 before blocking with block buffer (3% milk protein in PBS) for 1 hour at room temperature. Next, test agent or controls were loaded and incubated for 1 hour at room temperature. Plates were then washed three times with PBS/0.05% Tween 20. Finally, HRP conjugated secondary antibody, diluted 1/5,000 in block buffer, was added and incubated for 1 hour at room temperature. Plates were again washed three times with PBS/0.05% Tween before addition of TMB substrate and incubation for 6 minutes at room temperature. Subsequently, the reaction was stopped with stop solution. Absorbance was read at 450 nm and 570 nm. Absorbance at 570 nm was subtracted from absorbance at 450 nm for each plate.

### Cell Binding of LB Proteins

Jurkat cells were harvested, counted and washed once with PBS, before staining with Zombie UV viability dye diluted 1 in 1000 in PBS for 30 minutes at room temperature (RT). Induction was determined by calculating the ratio of the sample RLU/RLU in the absence of antibody following subtraction of background luminescence signal. Cells were washed with staining buffer (0.1% BSA in PBS), resuspended in staining buffer and separated into test aliquots. Cells were incubated with LB proteins at the indicated times for 30 min on ice. Cells were washed with staining buffer twice, before incubating with secondary antibody, or staining buffer alone, for 60 minutes on ice. Following secondary antibody incubation, cells were washed twice with staining buffer before fixing with 4% PFA for 10 minutes at RT. Fixed cells were resuspended in staining buffer and stored at 4° C. prior to analysis using the BD LSR Fortessa flow cytometer.

### MLR Assay

PBMCs were retrieved from cryogenic storage and thawed in serum free medium. Monocytes were isolated by positive magnetic separation and seeded at 2×106 cells/mL in CellGenix DC medium supplemented with gentamycin (50 μg/mL), human granulocyte-macrophage colony-stimulating factor (GM-CSF) (100 μg/mL) and human interleukin-4 (IL-4) (100 μg/mL). Cells were allowed to differentiate into iDCs for five days at 37° C., 5% CO2. At day five, MDA-MB-231 cells were harvested and irradiated at 25 Gy. After irradiation, cells were washed 3 times with total RPMI. Cells were then counted, plated at 10000 cells/well in a 96-well plate and allowed to settle for at least 1 h at 37° C., 5% CO2. Also at day 5, fresh PBMCs were retrieved from cryogenic storage and thawed in serum free medium. Allogenic CD3+ T cells were isolated by negative magnetic separation. iDCs were harvested and co-cultured in the 96-well plate, containing MDA-MB-231 cells, with the CD3+ T cells at a 1:10 ratio in total RPMI, in the presence or absence of test compounds and their relevant controls. The co-culture was incubated for two to five days at 37° C., 5% CO2. After two (IL-2 measurement) and five days (IFN-γ measurement) of incubation with test and/or control compounds, plates were centrifuged at 800 g for 5 min at 4° C. and supernatants were transferred to V-bottom plates. These plates were subsequently centrifuged at 800 g for 5 min at 4° C. and supernatants were transferred to new V-bottom plates. The plates were sealed and stored at −80° C. until further use. IL-2 and IFN-γ concentrations of the supernatants were measured by ELISA using the LEGEND MAX™ Human IL-2 kit and LEGEND MAX™ Human IFN-γ kit, respectively, with pre-coated plates according to Manufacturer's instructions. Primary T cell killing MDA-MB-231 Respective tumor cells were counted and seeded into the appropriate 96-well plates and left to adhere for approximately 24 h. PBMC cells were isolated from whole blood by centrifugation followed immediately by T cell isolation. To this end, PBMCs were incubated with a Pan T cell MicroBead cocktail and subsequently applied to a MACS separator column. Flow through cells representing enriched T cells were collected and diluted to desired levels. For the Incucyte® live cell analysis platform, LPD proteins or buffer with appropriate concentrations of Annexin V stain were added to adherent tumor target cells followed immediately by T cells and placement of plates into the Incucyte® S3 Live-Cell Analysis System incubator. Scanning intervals were set at every 4 h for 150 h. Annexin V data was normalized against Buffer/T-cell/Tumor cell signals before plotting.

### Cytokine Release Assay-Solid Phase Assay

The test samples and two controls provided were used in the plate bound assay at 1 μg/well. PHA (Sigma Aldrich, Poole, UK) and Anti-CD3 (OKT3) (Biolegend, San Diego, USA) were used as positive controls (final assay concentration 10 μg/mL and 1 μg/well respectively).

Negative control cultures were established in which PBMC cultures were treated with PBS. Clinical antibodies, TGN1412 (Evitria, Zurich, Switzerland) and Erbitux® (Eli Lilly, Indianapolis, USA), were used at final assay coating density of 1 μg/well. 60 μL of each antibody control or sample was added into a 96-well polypropylene plate (Corning, Amsterdam, The Netherlands) in duplicate and incubated for 2 hours at room temperature. Following incubation, plates were washed with 2×200 μL PBS followed by 200 μL media to remove any unbound antibodies. 100 μL culture media was added to the wells or media containing PHA to the relevant control wells, along with 100 μl freshly isolated PBMC at 2×10⁶ cells/mL. Plates were then incubated at 37° C. with 5% CO₂. (Method based on that used in Eastwood et. al 2013). Supernatants were taken from the cultures after 72 hours incubation and stored at −80° C. until use. Supernatants were analysed for cytokine levels using magnetic Milliplex kits (Merck Millipore, Hertfordshire, UK), according to the manufacturer's instructions. Supernatants, standards serially diluted five-fold and quality controls in culture media were incubated with pre-mixed capture beads for 16-18 hours followed by additional incubation with detection reagents. Data were acquired using a FLEXMap 3D® (Bio-Rad, Hertfordshire, UK). Cytokine secretion levels were determined in pg/ml from a standard curve using xPONENT® software (Bio-Rad). The mean of the sample and control duplicates were utilised for the analysis, in which the LQC and HQC (determined as the lowest and highest quantifiable calibrator) were used as the detection limits, with any values below and above the limits being replaced with the LQC and HQC values, respectively.

### PBMC Cytokine Release Assay

Cryopreserved PBMCs were thawed, counted and resuspended in the culture medium to a suitable concentration. Next, the cells were seeded at 2×105 cells per well into 96-well round-bottom plates containing the 5-fold concentrated test samples as well as the controls. The cells were kept in culture for 24 h at 37° C. and 5% CO2. After 24 h, the plates were centrifugated at 300 g for 10 minutes (min) at room temperature (RT) and supernatant was harvested and transferred into new 96-well, non-sterile, V-bottom plates. After a second centrifugation at 800 g for 3 min at RT of each plate, supernatant was again harvested and transferred into a different 96-well non-sterile V-bottom 96-well plates and stored at −80° C. until further analysis. Cytokine release assay: soluble incubation of test agents.

### PBMC Restore Cytokine Release Assay.

Cryopreserved PBMCs were thawed, counted and resuspended in the culture medium to a suitable concentration. Next, the cells were seeded at 1.5×107 cells per well into 24-well plates and incubated for 48 h at 37° C. and 5% CO2. After the pre-incubation, PBMCs were harvested, counted and seeded at 2×105 cells per well into 96-well round-bottom plates containing the 5-fold concentrated test samples as well as the controls. The cells were kept in culture for 24 h at 37° C. and 5% CO2. After 24 h, the plates were centrifugated at 300 g for 10 minutes (min) at room temperature (RT) and supernatant was harvested and transferred into new 96-well, non-sterile, V-bottom plates. After a second centrifugation at 800 g for 3 min at RT of each plate, supernatant was again harvested and transferred into a different 96-well non-sterile V-bottom 96-well plates and stored at −80° C. until further analysis. Cytokine release assay: soluble incubation of test agents.

### Example 2: Results and Discussion

### Protein Construct Design Cloning, Expression, and Characterization

To produce proteins for functional testing, DNA cassettes for each construct type were designed using combinations of the variable domain, constant domain and linker sequences found in Table 1. These CD28 humanized variants were then combined with PD-L1 variable domains, constant domains and linkers to form full-length heavy and light chain sequences (Table 4). Using these full-length chains, 2 initial designs were synthesized and cloned into expression vectors encoding human IgG1 heavy and light chain sequences and a free hinge-Fc fragment (asymmetric one arm constructs) (Table 5 and FIG. 1). Additional designs were synthesized and cloned into expression vectors encoding human IgG1 heavy and light chain sequences to create symmetric one arm or two arm constructs resembling a more classical IgG phenotype (Table 5, FIGS. 1A and B). Two CD28 antibody sequences were expressed as Fabs, termed Fab_001 (CD28 TGN derived sequence) and Fab_002 (Table 2) and IgG termed IgG_001 and IgG_002 (Table 3). Fab 001 and Fab_002 were tested for CD28 binding in surface plasmon resonance (FIG. 3). Fab_001 was confirmed to bind human/cyno recombinant CD28 with high affinity of KD 0.59 nM. Fab_002 showed similar high affinity to human/cyno recombinant CD28 protein (KD 0.53 nM). Neither protein showed binding to mouse CD28 protein as expected. IgG_001 and IgG_002 were subsequently tested for binding to coated human/cyno CD28 protein in ELISA (FIG. 4). As shown in Biacore experiments, both IgG_001 and IgG_002 showed similarly high binding affinity to human/cyno CD28 but no binding to mouse CD28. IgG1 isotype control showed no binding. TGN1412 (IgG4 isotype) showed overlapping binding profiles to IgG_001 confirming that removal of an unpaired cysteine as well as IgG1 isotype does not affect binding affinity of IgG_001.

Next, the LPD proteins described in Table 5 were produced by transient transfection of CHO cells, then purified by ProA, IEX and/or SEC. After the ProA step, proteins were examined for yield and uniformity by SEC (Table 6). Fully purified proteins demonstrated high purity (>95%) and uniformity by analytical SEC, demonstrating that the best-behaved constructs can be expressed and purified as an intact, stable product, in a single process. Symmetric one arm construct showed a significantly lower purity after the initial ProA purification step (LPD28-6 to LPD28-9, Table 6).

TABLE 6
Productivity analyses for selected proteins

| | Main | HMW | LMW |
|---|---|---|---|
| Name | Product (%) | (%) | (%) |
| LPD28-1 | 79 | 9 | 12 |
| LPD28-2 | 77 | 6 | 17 |
| LPD28-3 | 55 | 23 | 21 |
| LPD28-4 | 49 | 26 | 25 |
| LPD28-5 | 44 | 22 | 34 |
| LPD28-6 | 21 | 74 | 5 |
| LPD28-7 | 27 | 70 | 3 |
| LPD28-8 | 27 | 71 | 2 |
| LPD28-9 | 26 | 71 | 3 |
| LPD28-10 | 75 | 23 | 2 |
| LPD28-11 | 75 | 23 | 2 |
| LPD28-12 | 67 | 33 | 0 |
| LPD28-13 | 82 | 28 | 0 |

HMW = Higher Molecular Weight Product
LMW = Lower Molecular Weight Product

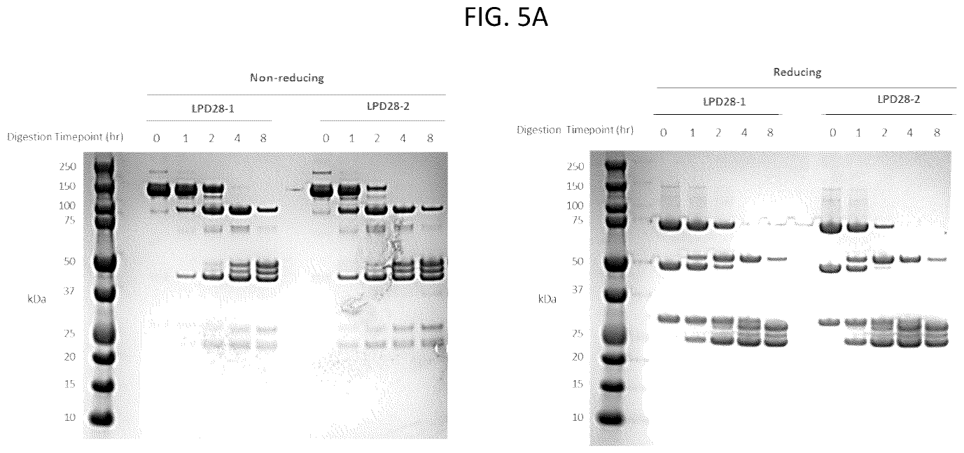

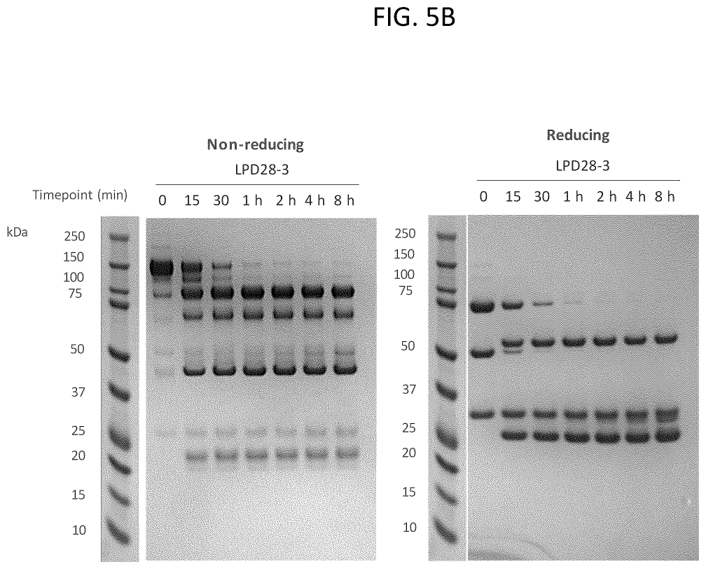

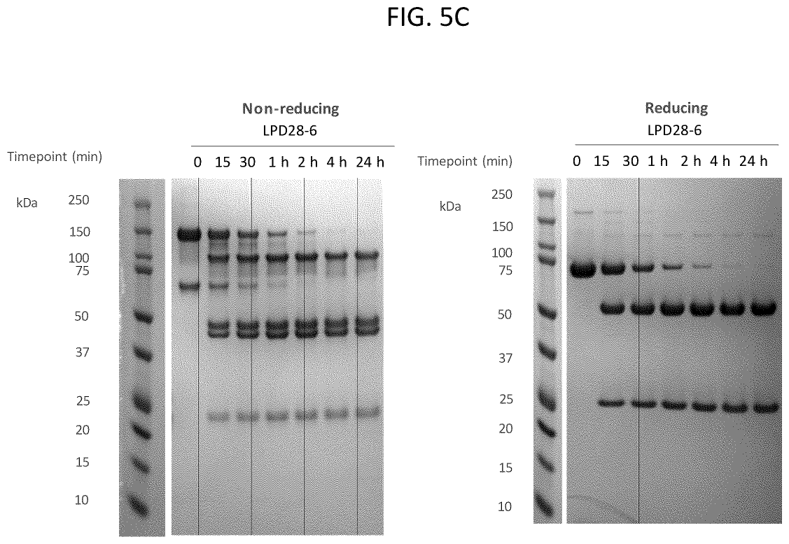

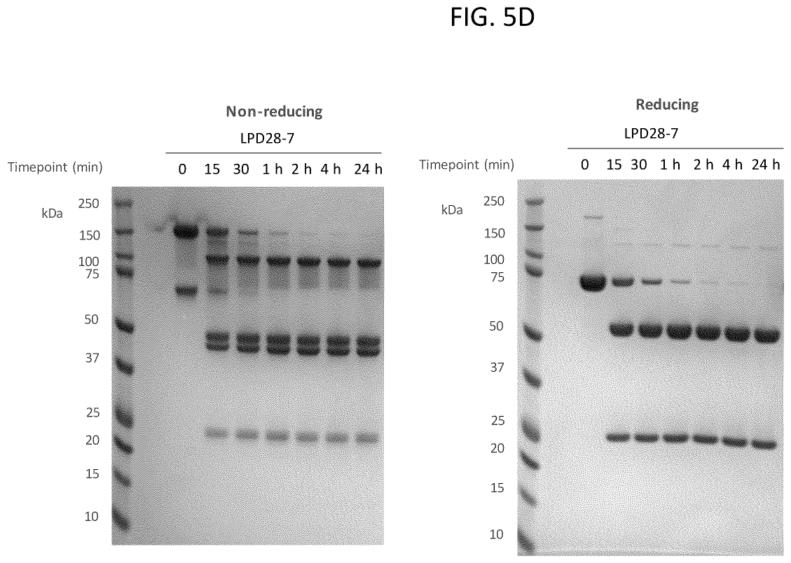

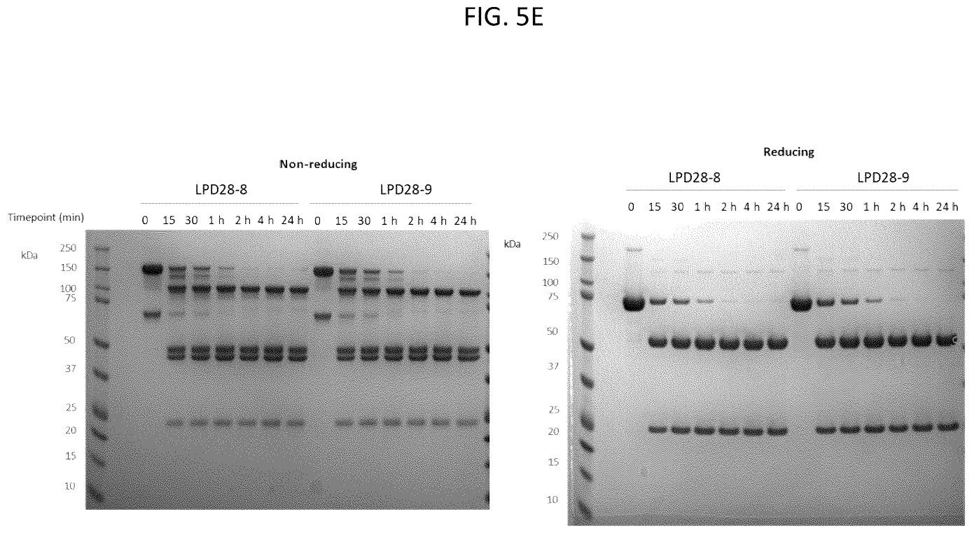

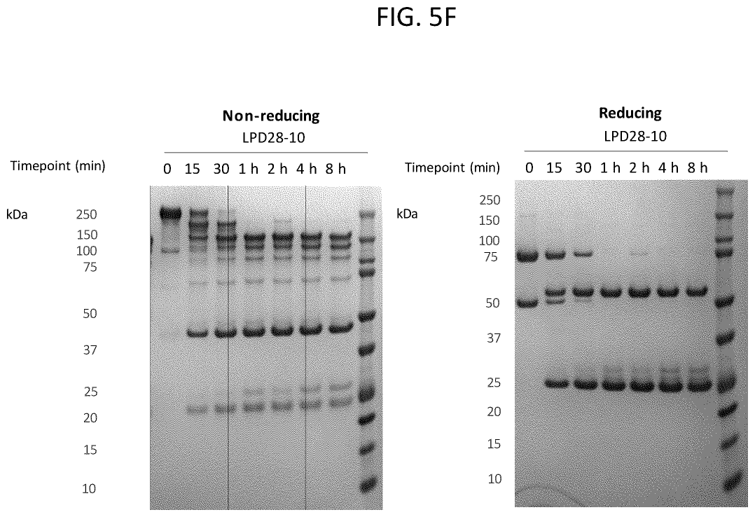

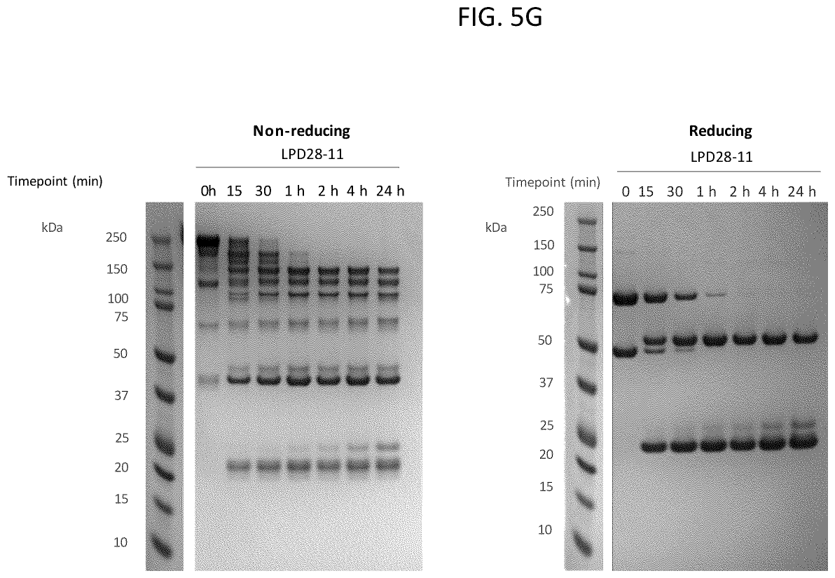

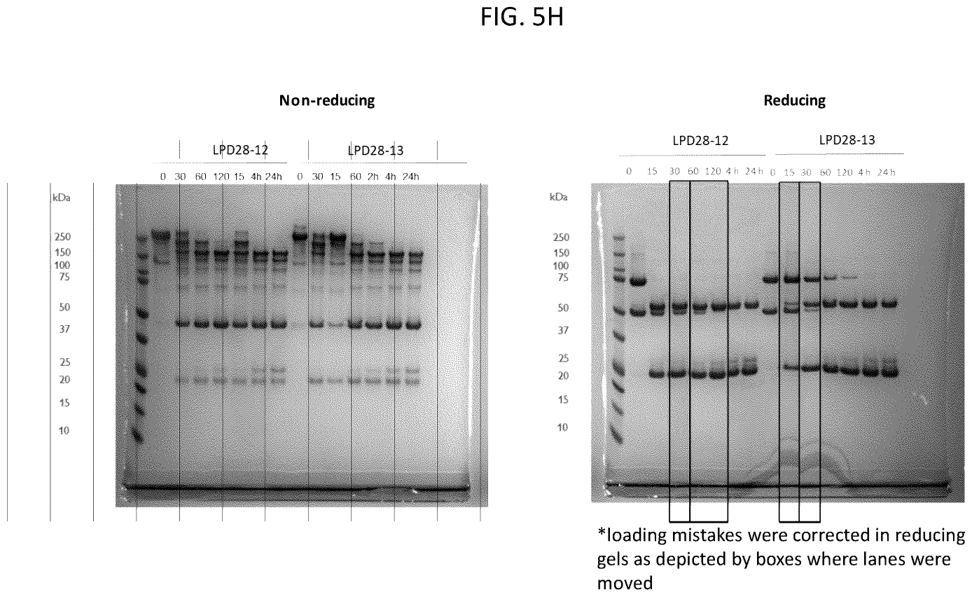

Uniform and fully purified molecules were incubated at 37° C. for the indicated times in the presence or absence of human MMP12 enzyme and digestions profiles were assessed using SDS-PAGE under reducing and non-reducing conditions (FIG. 5A to FIG. 5H). FIGS. 5A and B show digestion profiles of exemplar asymmetric one arm LPD proteins (LPD28-1, LPD28-2 and LPD28-3). LPD28-1 and LPD28-2 were treated with full length MMP12 enzyme while LPD28-3 and following molecules were digested with catalytic domain MMP12 enzyme. Nondigested (intact) proteins showed the expected band patterns in reducing and non-reducing SDS PAGE with one predominant band at 150 kDa visible in non-reducing, and 3 chains visible at 75 kDa (LB heavy chain), 50 kDa (LB light chain) and ~30 kDa (Fc Stump) in reducing gels (FIGS. 5A and B). Upon incubation with MMP12 at 37° C., marked differences in band distribution compared to undigested proteins suggested cleavage of both LB heavy chains and LB light chains. In reducing SDS-PAGE, the appearance of multiple 25-30 kDa bands (corresponding to cleaved V-C domain from either LB light or heavy chain, and proteolytically-released single chain Fc fragments) and ~55 kDa band (corresponding to VH-CH domain with Fc), and corresponding disappearance of intact LB light and heavy chain products at 75 and 50 kDa, demonstrates cleavage of chains in their LHL linkers, thereby creating the respective products as depicted in FIG. 5A and FIG. 5B.

Further MMP12 enzyme digests and SDS-PAGE analyses of additional LPD protein modalities (FIGS. 5C-H) demonstrated a different pattern reflecting their respective designs: Symmetric one arm construct digests are depicted in FIGS. 5C, D and E and symmetric two arm LPD proteins are depicted in FIGS. 5F, G, H. Undigested symmetric one arm LPD proteins showed the expected band patterns in reducing and non-reducing SDS PAGE with one predominant band at 150 kDa visible in non-reducing SDS-PAGE. As expected, only one chain is visible at 75 kDa in contrast to asymmetric LPD molecules (LB heavy chain and LB light chain) in reducing SDS-PAGE gels (FIGS. 5C, D, E). Upon incubation with MMP12 at 37° C., marked differences in band distribution compared to undigested proteins suggested cleavage of 75 kDa sized bands. In reducing SDS-PAGE, the appearance of 25-30 kDa bands corresponded to cleaved V-C domain from either LB light or heavy chain. In addition, this is accompanied by appearance of a ~55 kDa band corresponding to VH-CH and VL-CH domains with Fc. Undigested (intact) LPD light and heavy chain products at 75 kDa disappear with increasing MMP12 treatment times, demonstrating cleavage of either chain in both of the LHLs, thereby creating the respective products as depicted in FIGS. 5C, D and E. The selected symmetric one arm LPD proteins shown in FIG. 5C were designed to contain different hinge linkers: either LHL in combination with LHL-F (LPD28-6) or LHLX in combination with LHLX-F (LPD28-7) (FIG. 5C). In the symmetric one arm format, there is no discernible difference in susceptibility to MMP12 cleavage of LPD proteins containing either LHL in combination with LHL-F (LPD28-6) or LHLX LHLX-F (LPD28-7) (FIG. 5C). The selected symmetric one arm LPD proteins shown in FIG. 5D were designed to contain LPD hinge linkers LHL in combination with LHL-FR, with the LHL-FR appearing either in the VH chain (LPD28-8) or VL chain (LPD28-9) (FIG. 5D). LHL-FR was designed to theoretically show higher susceptibility to MMP cleavage. In the symmetric one arm format, there is no discernible difference in susceptibility to MMP12 cleavage of LPD proteins containing LHL-FR in either the VL or VH chain (FIG. 5D).

Undigested two arm LPD proteins showed the expected band patterns in reducing and non-reducing SDS PAGE with one predominant band at 250 kDa visible in non-reducing, and two chains visible at 75 kDa (LB heavy chain) and 50 kDa (LB light chain) in reducing gels (FIGS. 5F, G, H). Upon incubation with MMP12 at 37° C., marked differences in band distribution compared to intact/undigested proteins suggested cleavage of both LPD heavy chains and LPD light chains. In reducing SDS-PAGE, the appearance of multiple 25-30 kDa bands corresponds to cleaved V-C domain from either LPD light or heavy chain. The appearance of a ~55 kDa band in reducing SDS-PAGE corresponded to VH-CH domain with Fc. Undigested LPD light and heavy chain products at 75 and 50 kDa disappear with increased MMP12 treatment time, demonstrating cleavage of either intact chain in both of the LHLs, thereby creating the respective products as depicted in FIGS. 5F, 5G, and 5H. FIG. 5F and FIG. 5G depict two arm LPD proteins that were designed with LPD linkers LHL in their LPD heavy chains and LHL-F in their LPD light chains but contain different CD28 V domains, TGN derived domains are present in LPD28-10 while FR104 domains are present in LPD 28-11. In the two-arm format (FIG. 1B, left), there is no discernible difference in susceptibility to MMP12 cleavage of LPD proteins containing different CD28 V domains (FIGS. 5F and 5G)

LPD28-12 and LPD28-13 (FIG. 5H) contain LPD linkers LHL and LHL-FR in either the LPD light chain (LPD28-13) or LPD heavy chain (LPD28-12). LHL-FR was designed to be cleaved relatively faster by MMP12 than LHL-F and LHL LPD linkers. The intact heavy chain in LPD 28-12 showed rapid disappearance at 15 min in contrast to LPD 28-10 and LPB28-11 and LPD28-13 that contain LHL LPD linkers. LPD28-13 retained significant amounts of 75 kDa LB heavy chain band at the same MMP12 treatment times. Comparing LPD28-12 and LPD28-13 disappearance of intact LPD light chain bands, LPD28-13 which contains the LHL-FR linker in its LPD light chain shows increased disappearance speeds compared to LPD28-11 that contains a LHL linker in its LPD light chain. Almost no intact LPD light chain is visible after 30 min of MMP12 treatment time in LPD28-13 reducing SDS-PAGE. In contrast, intact LPD light chains are clearly visible at 60 min in LPD28-12 reducing SDS-PAGE. This confirmed an increased susceptibility to MMP12 cleavage of the LHL-FR linker in two arm LPD proteins, regardless of whether the LHL-FR linker is present in LPD light or heavy chains.

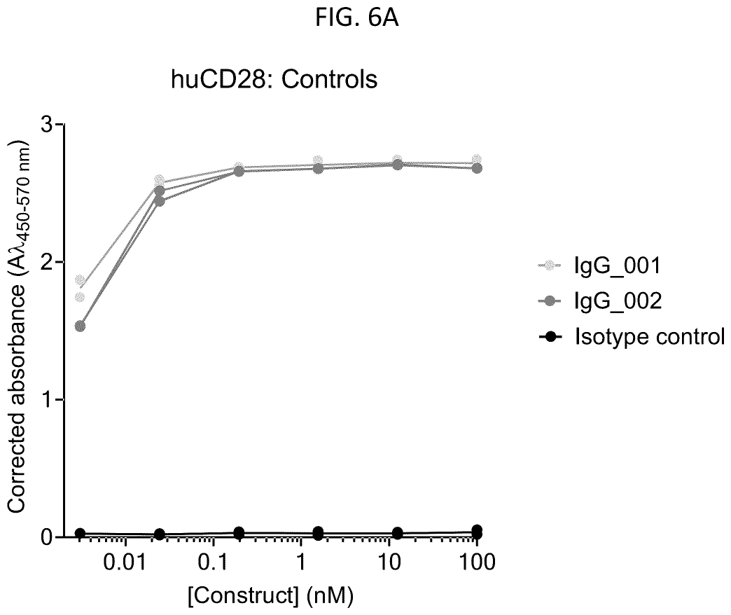

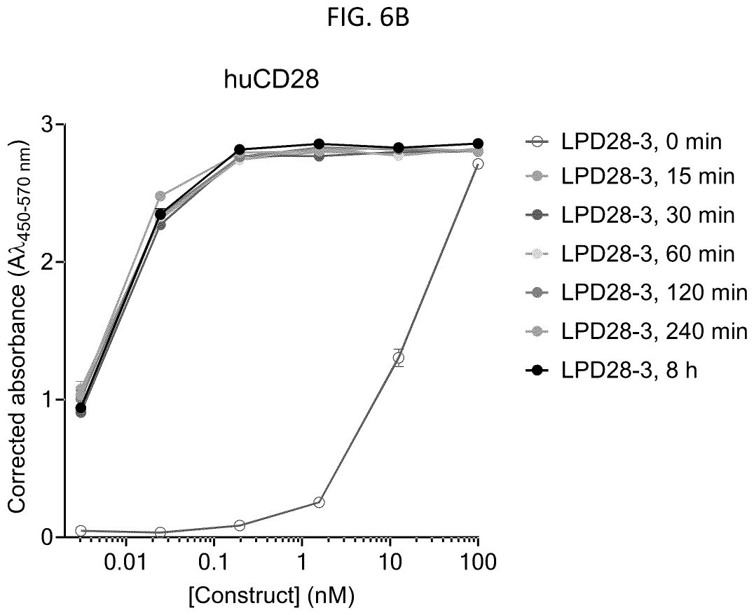

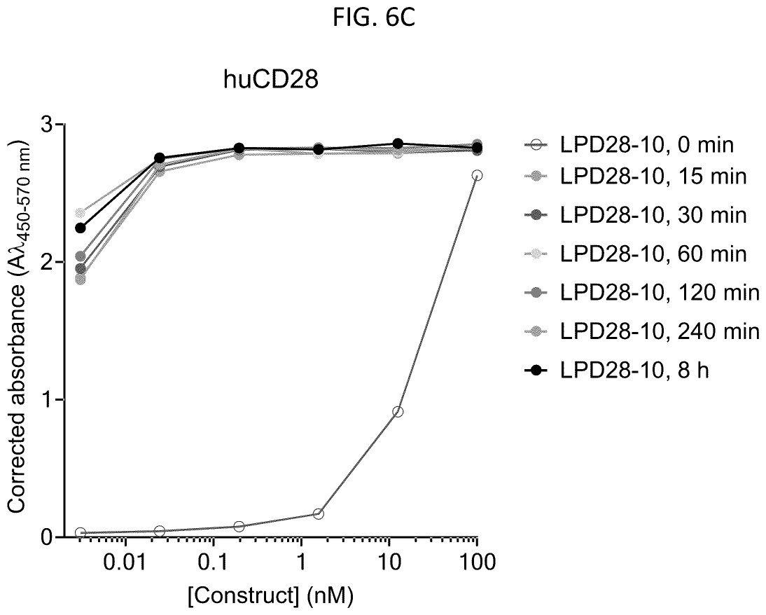

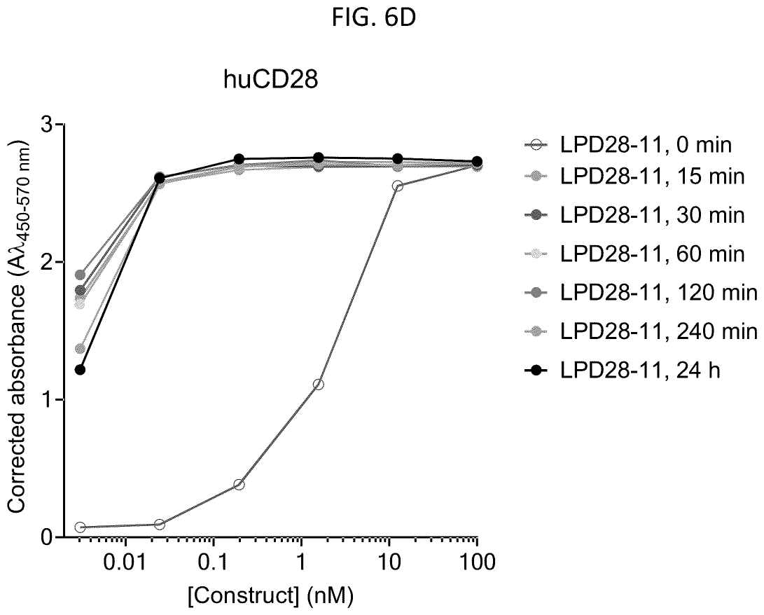

To assess the ability of undigested fully intact and MMP12 incubated proteins to interact with CD28, binding ELISAs to recombinant human CD28 domain were carried out. In parallel, purified IgG_001 and IgG_002 proteins were used as positive controls while isotype binding was used as negative control. As expected, both IgG_001 and IgG_002 showed strong binding signals to CD28 in ELISA while isotype control showed no binding (FIG. 6A). Interestingly, all exemplary proteins tested (LPD28-3, LPD28-10 and LPD-11; FIG. 6B-D) confirmed that CD28 binding was greatly diminished in fully intact proteins but strongly increased after incubation with MMP12 at 37° C. at the indicated times. Specifically, FIG. 6B shows ELISA binding of undigested (intact/0 min) or 15 min, 30 min, 1 h, 2 h, 4 h or 8 h MMP12 incubated LPD28-3 protein to human CD28. MMP12 treated LPD28-3 proteins bind to CD28 to high levels at all tested concentrations while undigested LPD28-3 shows significant CD28 binding signals at the two highest concentrations tested (12.5 and 100 nM) only. FIG. 6C shows ELISA binding of undigested (0 min) or 15 min, 30 min, 1 h, 2 h, 4 h or 8 h MMP12 incubated LPD28-10 protein to human CD28. MMP12 treated LPD28-10 proteins bind to CD28 to high levels similar to bivalent controls at all tested concentrations while intact LPD28-10 shows significant CD28 binding signals at the two highest concentrations tested (12.5 and 100 nM) only. FIG. 6D shows ELISA binding of undigested (0 min) or 15 min, 30 min, 1 h, 2 h, 4 h or 24 h MMP12 incubated LPD28-11 protein to human CD28. MMP12 treated LPD28-11 proteins bind to CD28 to high levels similar to bivalent IgG controls (FIG. 6A) at all tested concentrations while undigested LPD28-11 shows significant CD28 binding signals at the three highest concentrations tested (1.5, 12.5 and 100 nM) only (FIG. 6D). This data confirms that undigested/intact LPD28 molecules show severely impaired binding to recombinant CD28 but can be proteolytically activated to regain their full binding affinity.

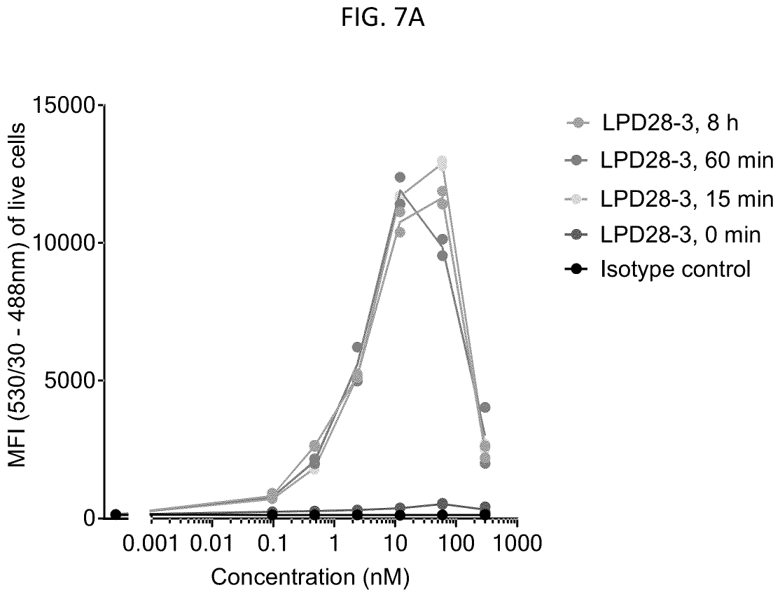

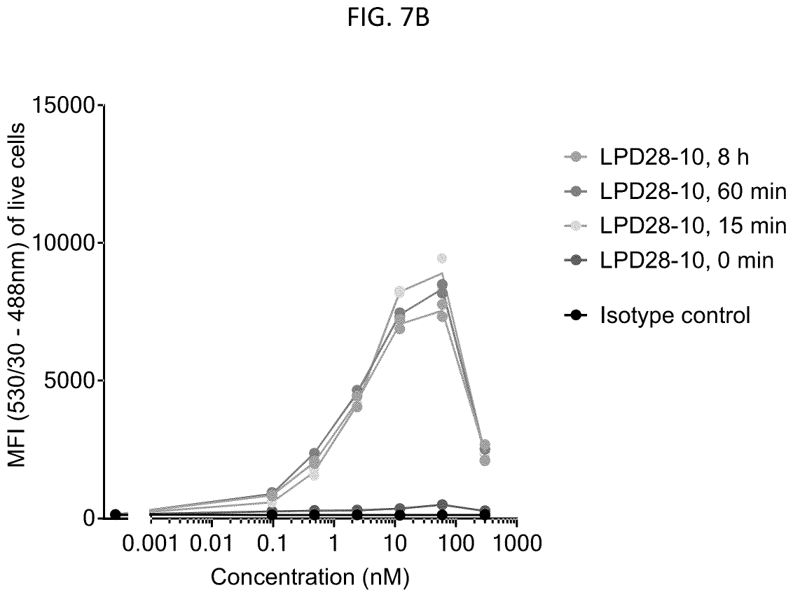

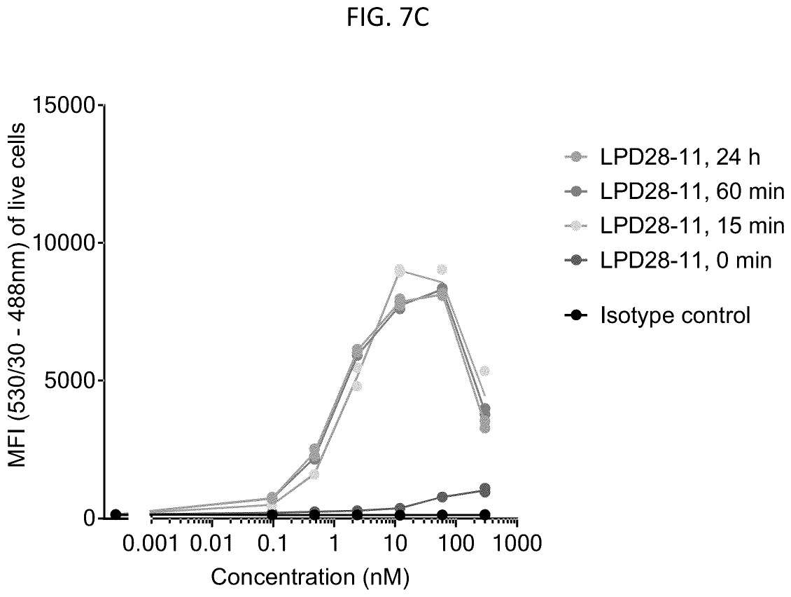

To further assess the ability of undigested fully intact and MMP12 incubated proteins to interact with CD28, Jurkat cell binding of selected proteins (LPD28-3, LPD28-10 and LPD-11; FIGS. 7A-C) was carried out. As shown previously with recombinant proteins, binding signals to Jurkat cells confirmed that CD28 binding in its native form displayed on the cell surface was greatly diminished in fully intact LPD proteins. Binding strongly increased after incubation with MMP12 at 37° C. at the indicated times. Specifically, FIG. 7A shows Jurkat cell binding of intact (0 min) or 15 min, 30 min, 1 h, 2 h, 4 h or 8 h MMP12 incubated LPD28-3 proteins. MMP12 treated LPD28-3 proteins bind to Jurkat cell CD28 to high levels at all tested concentrations while intact LPD28-3 showed no significant CD28 significant binding signals at any test concentration. FIG. 7B shows Jurkat cell binding of intact (0 min) or 15 min, 30 min, 1 h, 2 h, 4 h or 8 h MMP12 incubated LPD28-10 proteins. MMP12 treated LPD28-10 proteins bind to Jurkat cell CD28 to high levels at all tested concentrations while intact LPD28-10 showed no significant CD28 significant binding signals at any test concentration. FIG. 7C shows Jurkat cell binding of intact or 15 min, 30 min, 1 h, 2 h, 4 h or 24 h MMP12 incubated LPD28-11 proteins. MMP12 treated LPD28-11 proteins bind to Jurkat cell CD28 to high levels at all tested concentrations while undigested LPD28-11 showed some CD28 binding signals at the two highest tested concentrations only (60 and 300 nM). This confirmed the relatively higher CD28 binding signal observed for undigested LPD28-11 in ELISA (FIG. 6D). No cell binding for isotype control was detected (FIGS. 7A-C).

In order to show that CD28 V domains used in LPD proteins are functional and able to activate CD28+ Jurkat IL2 reporter cells as expected, purified IgG proteins were tested in this assay. FIG. 8 shows expected activation signals achieved with control proteins. Commercial TGN1412 (IgG4), IgG_001 and IgG_002 were incubated at indicated concentrations with and without CD3 costimulation. CD3 costimulation is provided by constitutive addition of 15 nM Anti CD3 antibody (SP34) to samples where indicated. As expected, luminescence signals could not be detected for IgG_002 (non superagonist) without CD3 stimulation. In contrast, TGN1412-IgG4 (superagonist) showed clear signal even in the absence of CD3 stimulation. IgG_001 contains a highly similar V domain to TGN1412 (IgG4) but was expressed as IgG1 Fc isotype. IgG_001 shows a clear signal in the absence of CD3 stimulation but lower than its TGN1412-IgG4 counterpart. It is noteworthy that CD28 binding ability of TGN1412 (IgG4) and IgG_001 in ELISA is indistinguishable. The importance and effects of Fc isotype for TGN mediated CD28 superagonism are well described. It is thought to largely relate to sterical differences of V domain display and relative rigidity of binding domains determined by the hinge sequences present in IgG4 vs IgG2 vs IgG1 Fc isotypes (Ball et al, J Immunol 2012). Both TGN1412 and IgG_002 also elicit luminescence signal in the presence of CD3 stimulation as expected.

Further, some exemplary LPD proteins with differing valencies, LHL linkers and CD28 V domain combinations were selected and tested for functionality in a mixed cell culture assay using a human PD-L1+ cell line MDA-MB-231 in combination with a Jurkat cell line engineered to provide reporter signal for human CD28 activation (IL2 promotor).

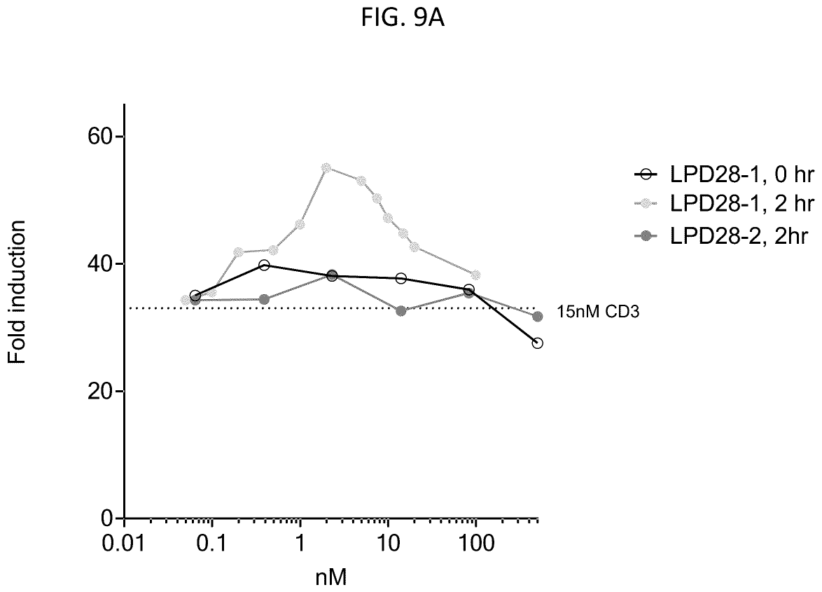

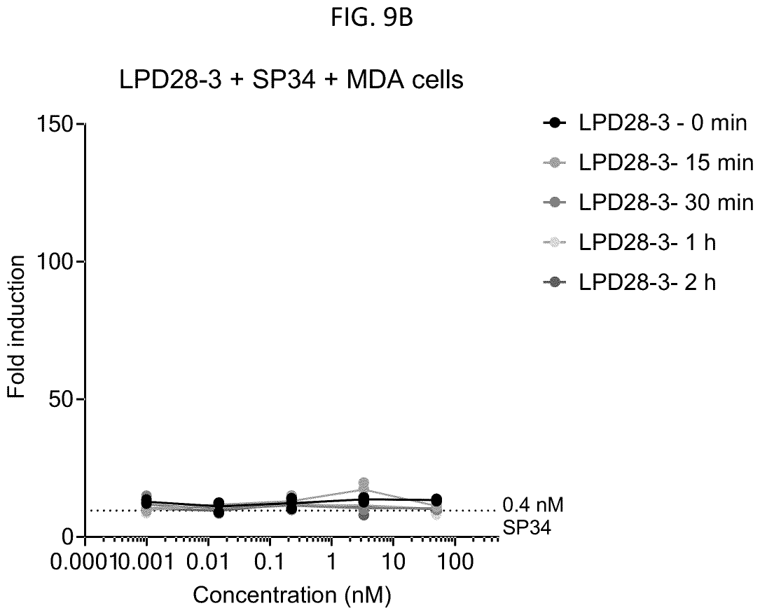

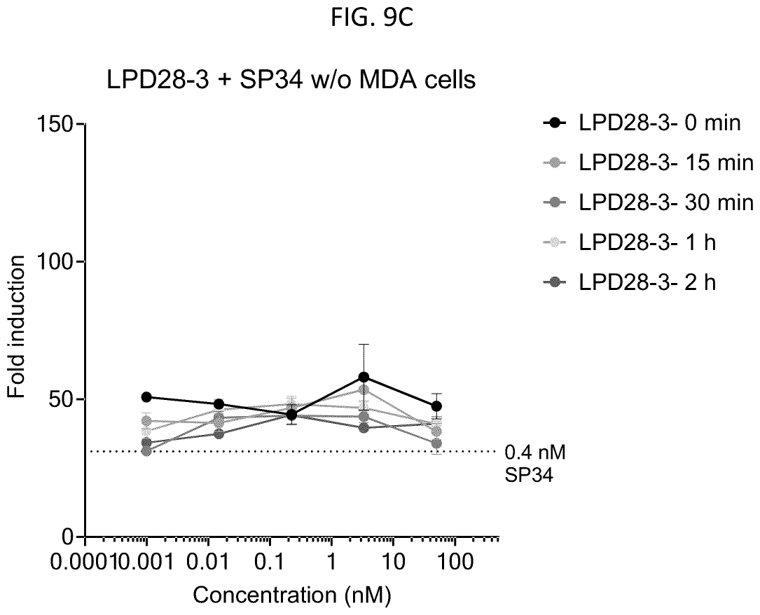

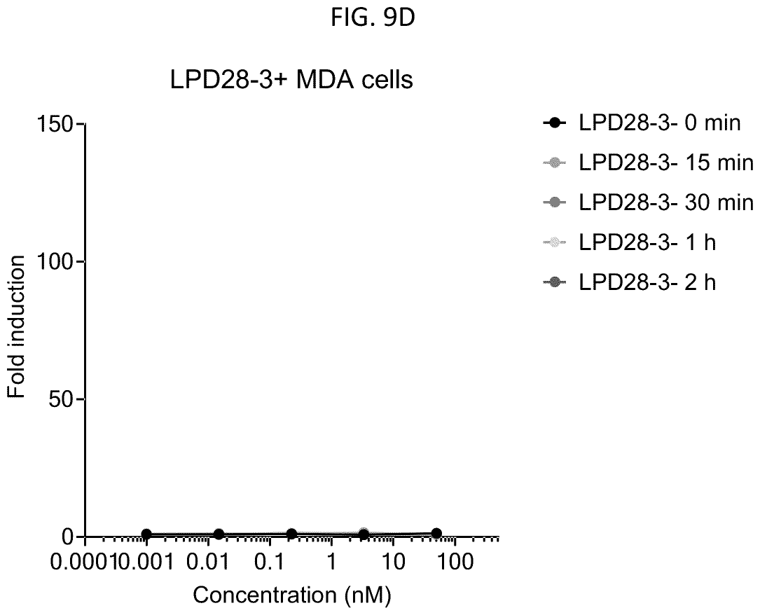

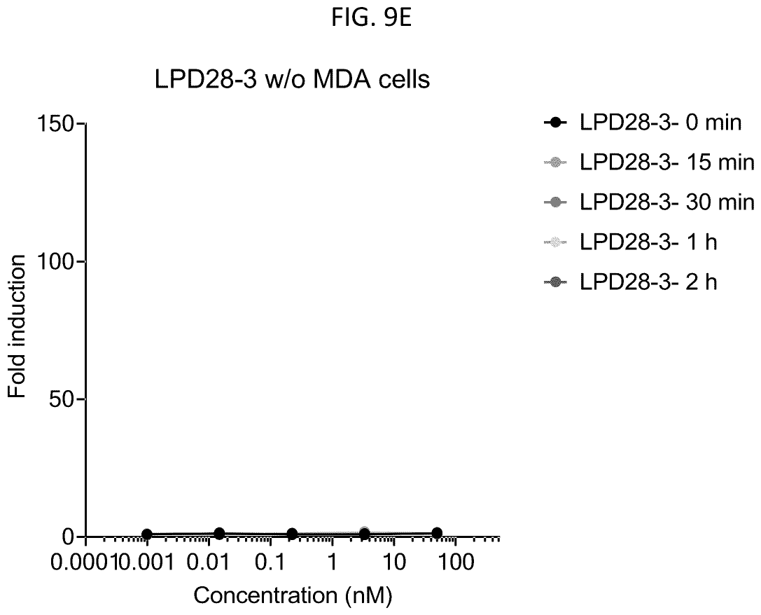

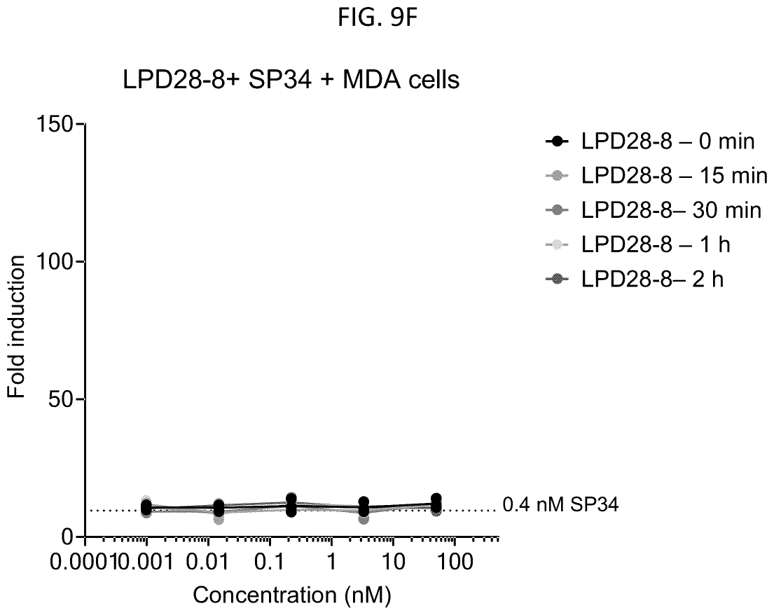

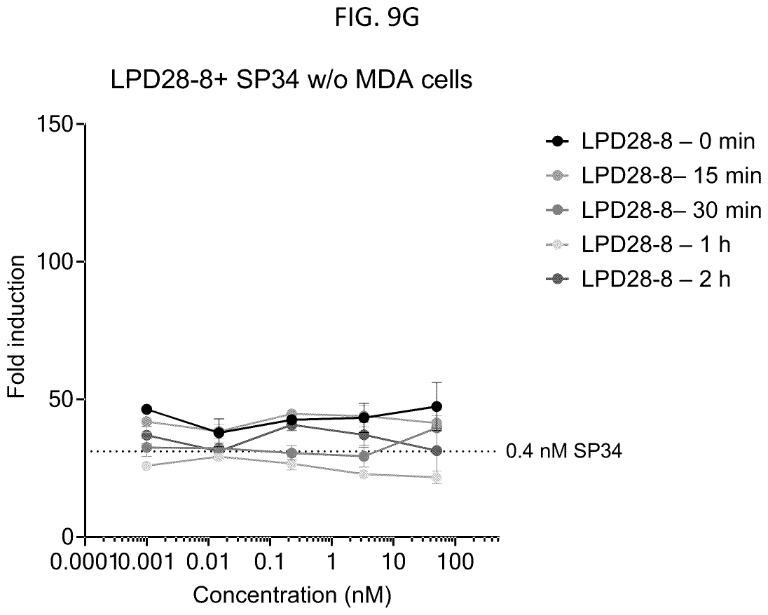

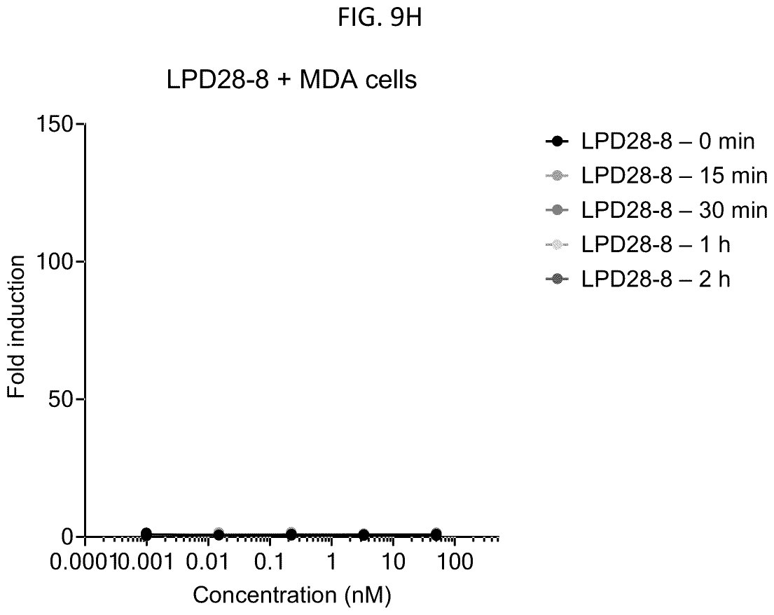

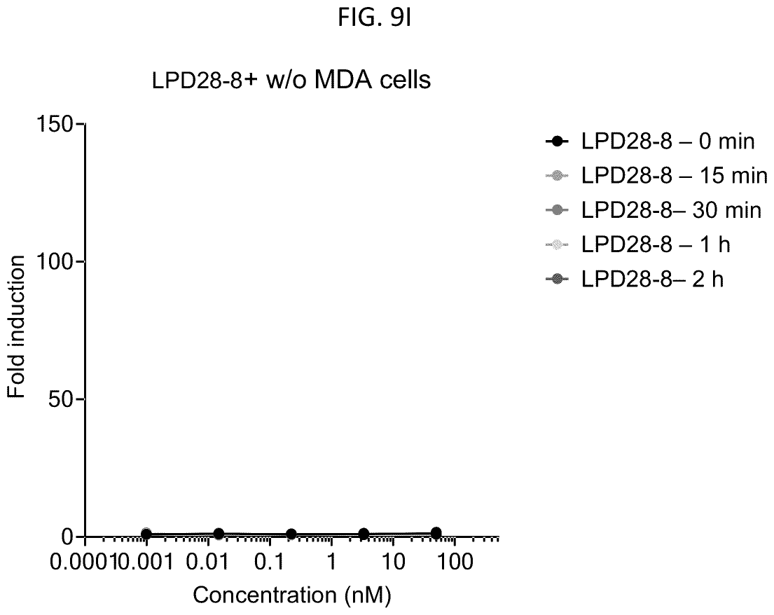

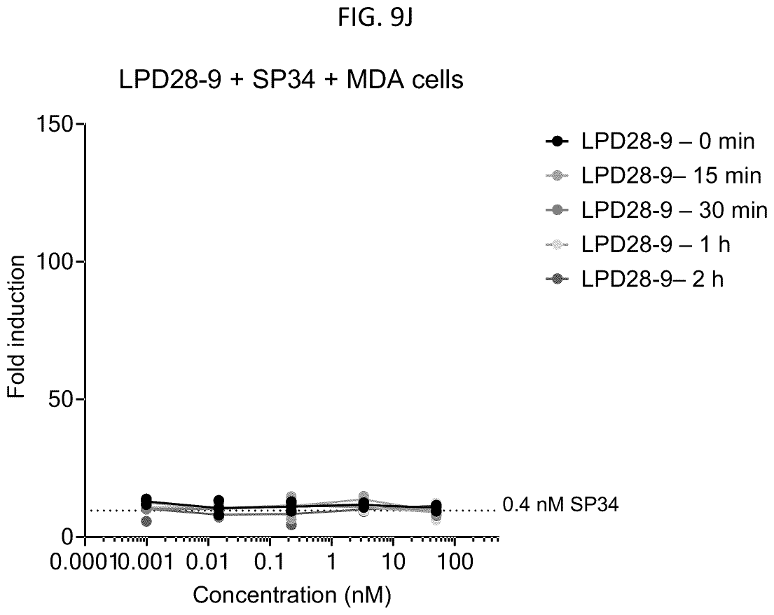

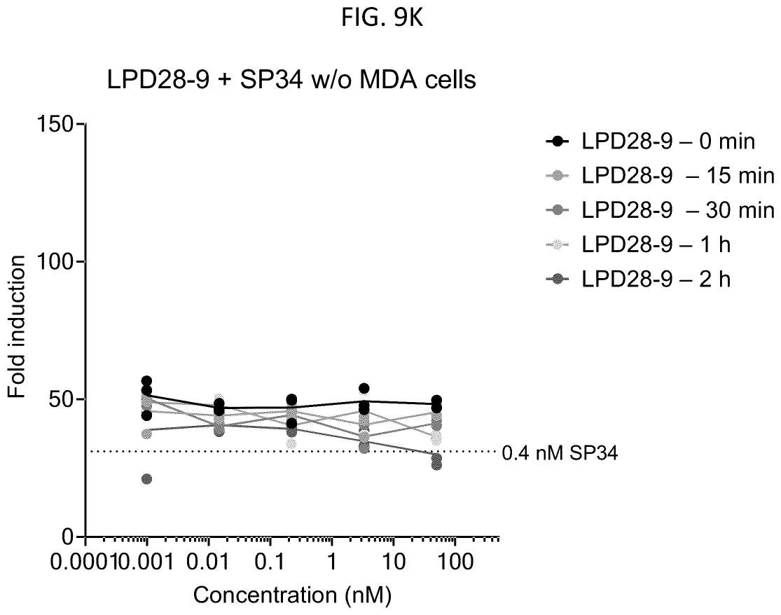

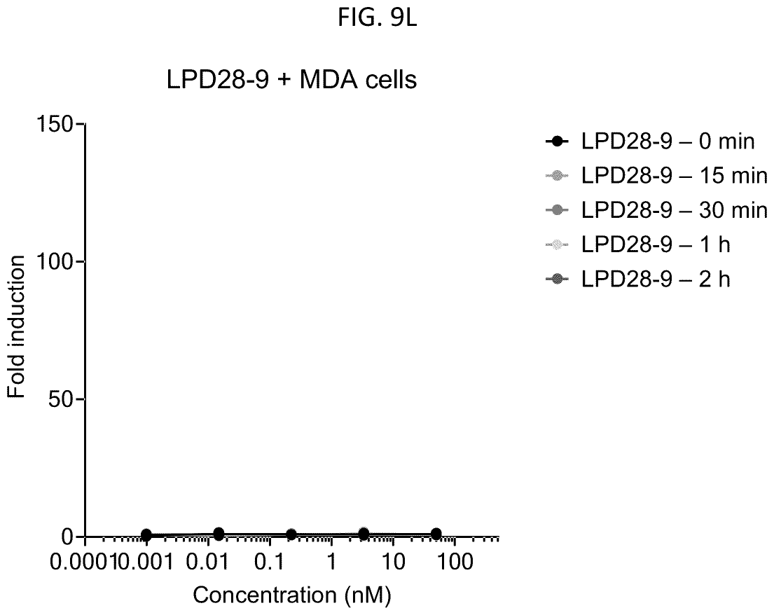

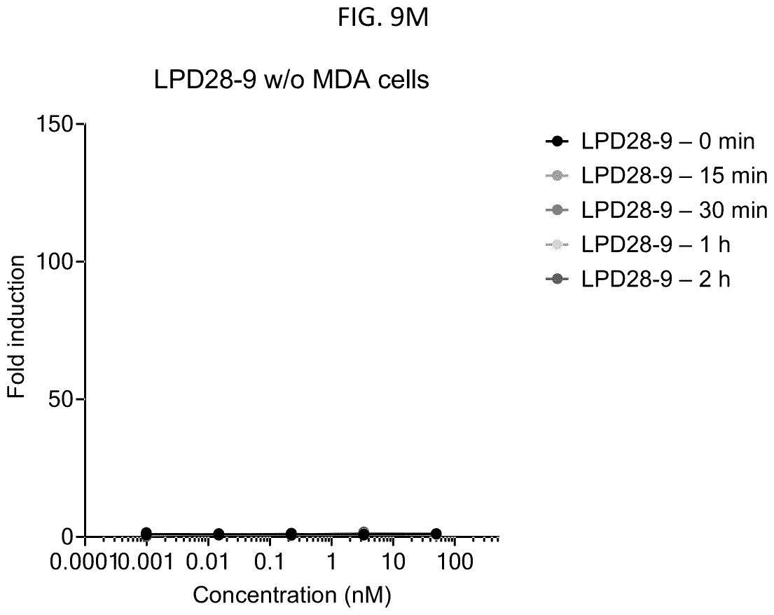

FIG. 9A shows fold induction of luminescence signals of 2 h MMP12 treated LPD28-1 and LPD28-2 in the presence of CD3 stimulation. LPD28-2 did not show significant induction of signal, while LPD28-1 had low signal for the highest concentrations but increased in activation signal with decreasing concentrations following a ‘hook effect’ or impairment of co-stimulation ability at high concentrations. This illustrates that CD28 activation is impaired in undigested/intact LPD molecules but can be restored by proteolytic cleavage.

Further, selected LPD proteins were tested for their ability to stimulate CD28 on Jurkat IL2 reporter cells in four assay conditions: in the presence and absence of a fixed CD3 stimulation signal in combination with the presence or absence of a human PD-L1+ cell line MDA-MB-231 and therefore PDL1 engagement.

LPD28-3 was not able to provoke any CD28 stimulatory signal, regardless of intact, MMP12 treatment or assay condition tested (FIGS. 9B-E).

Similarly symmetric constructs LPD28-8 and LPD28-9 are not able to provoke any CD28 stimulatory signal, regardless of condition tested or MMP12 incubation time (FIG. 9F-M). This shows that CD28 V domains, their epitope as well as LPD protein modality strongly influence their CD28 activating ability.

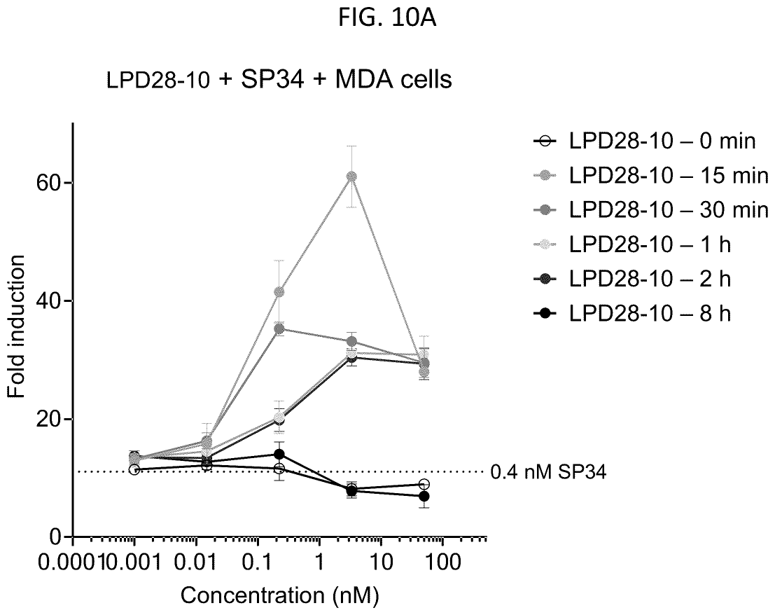

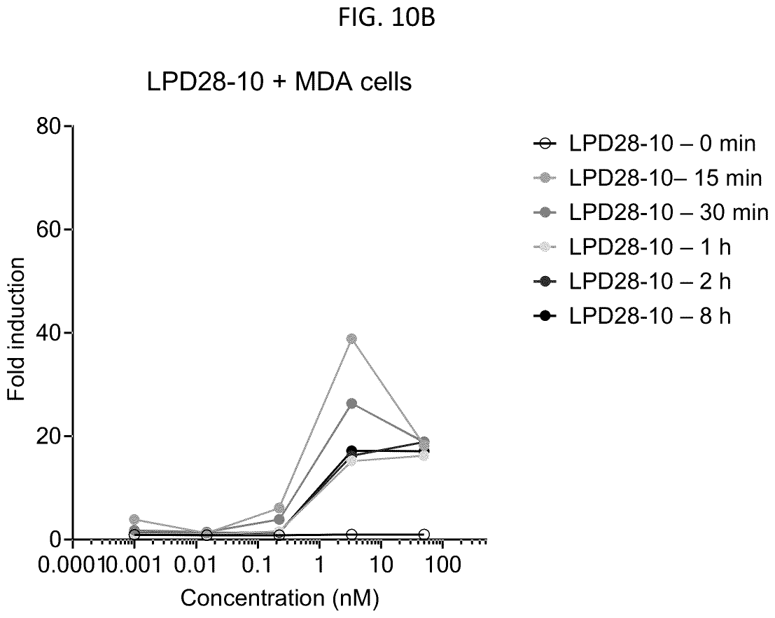

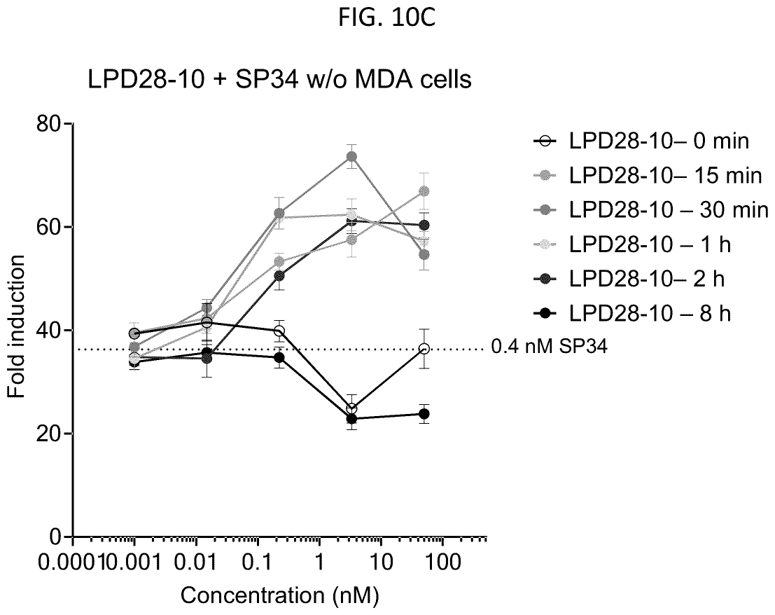

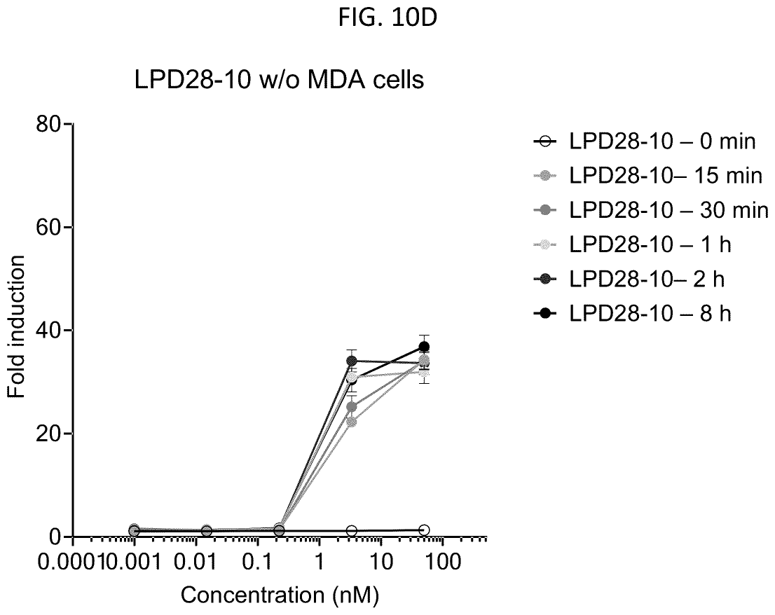

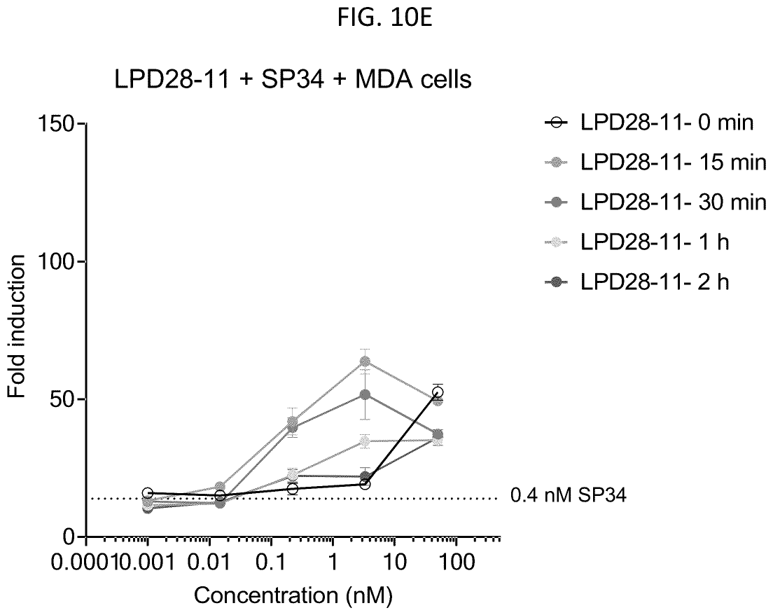

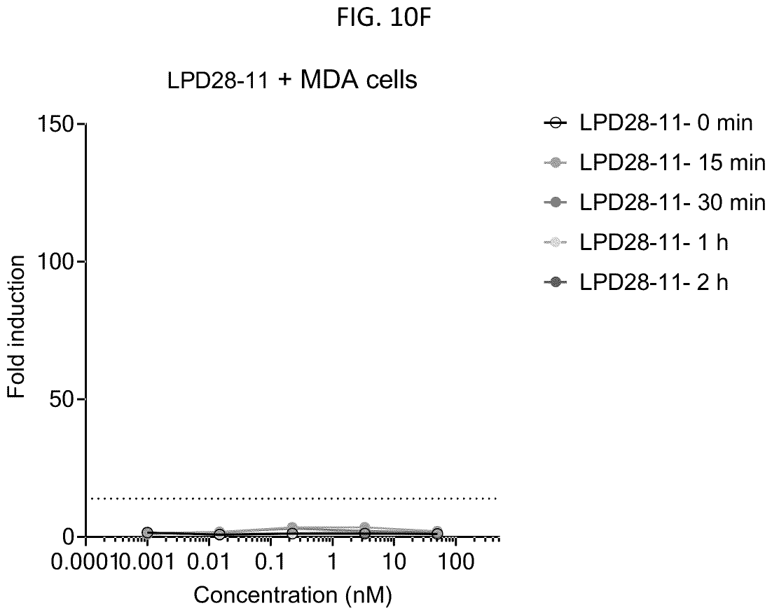

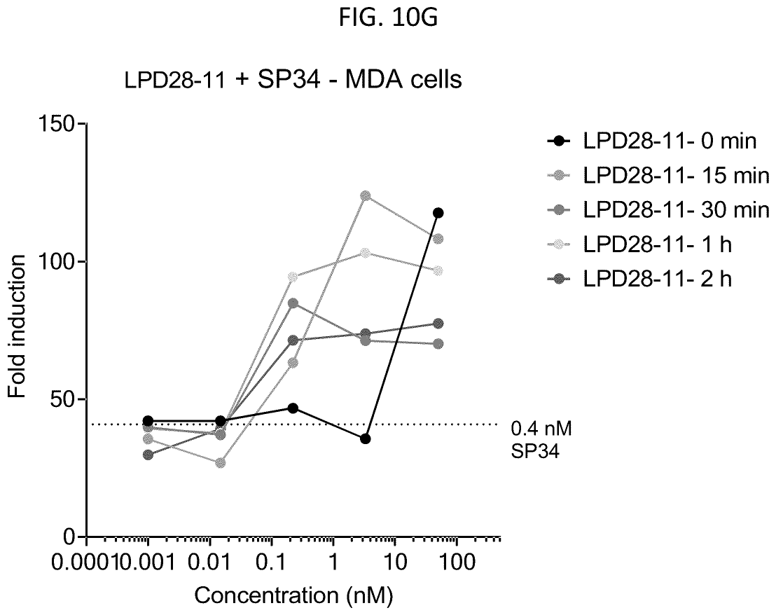

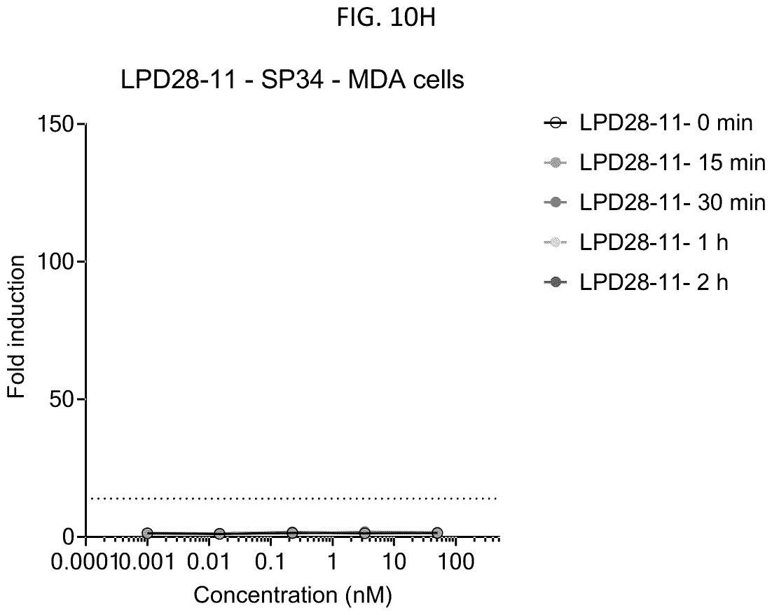

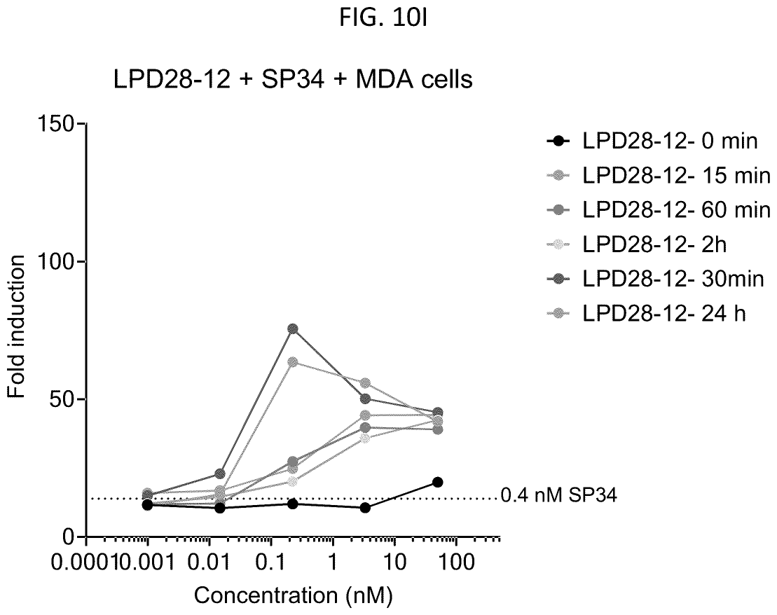

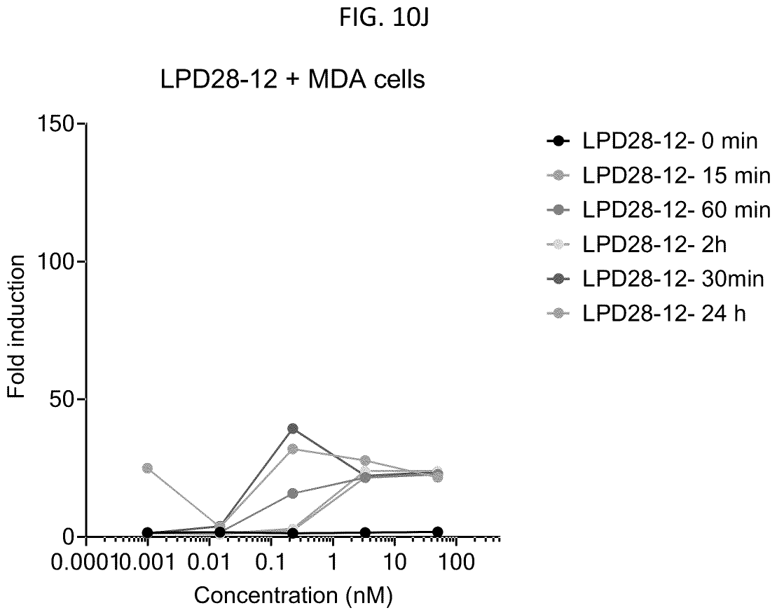

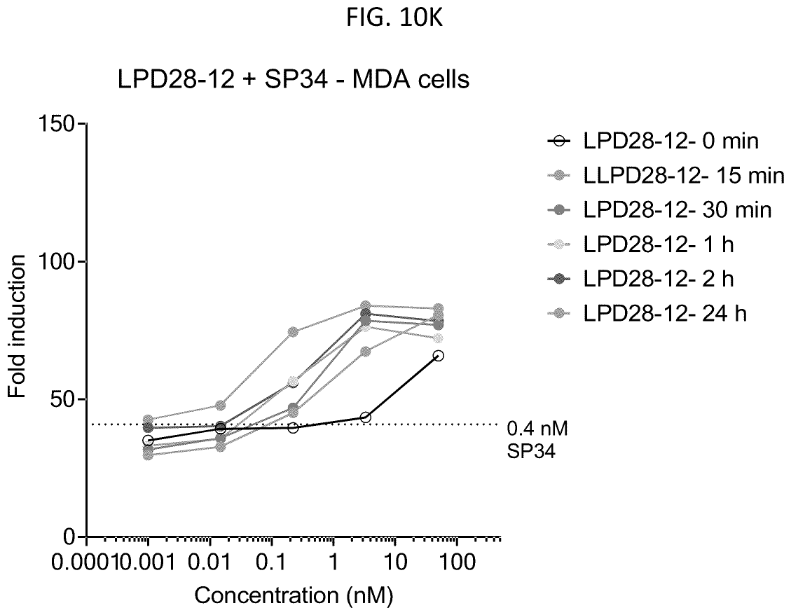

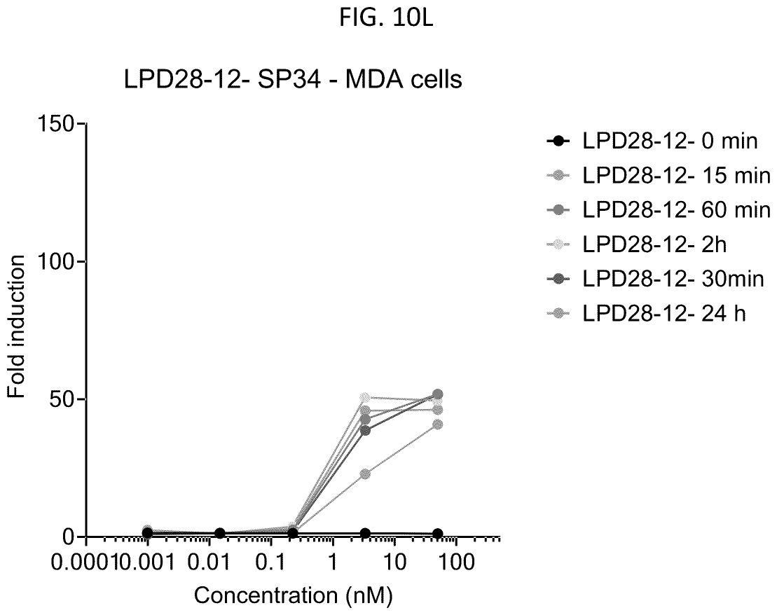

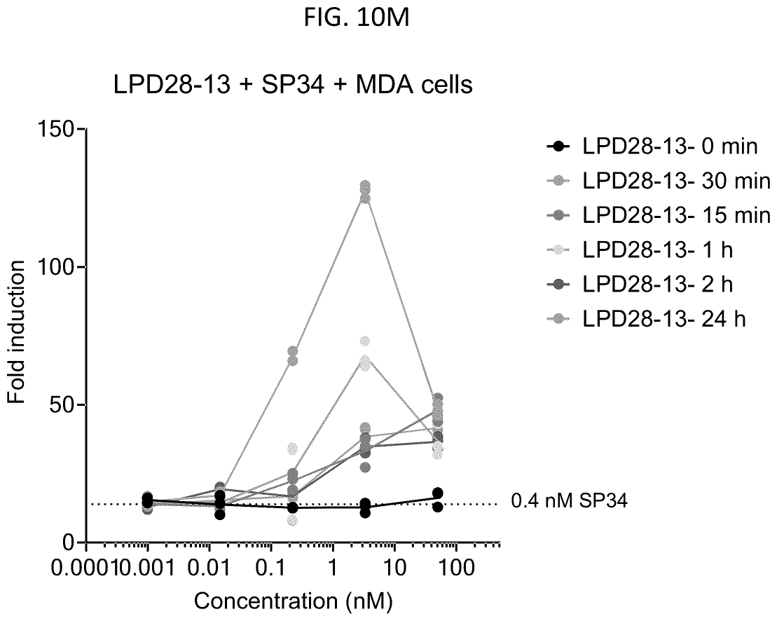

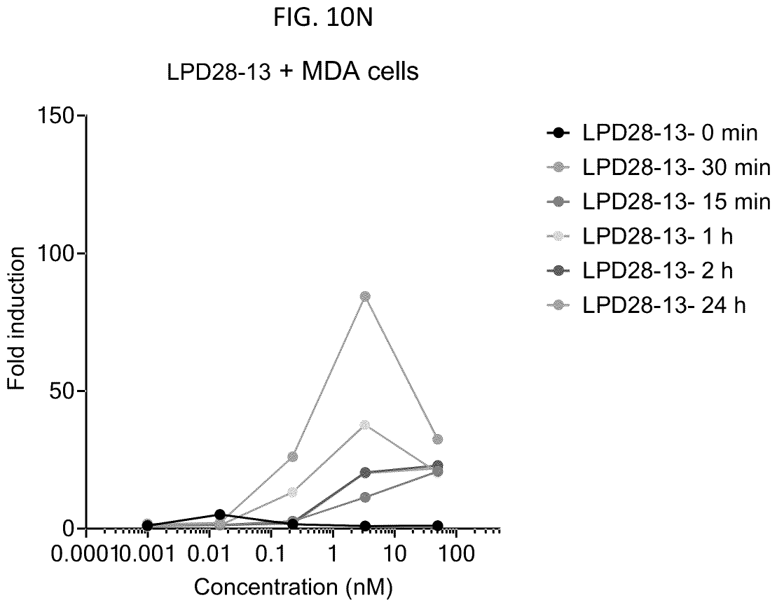

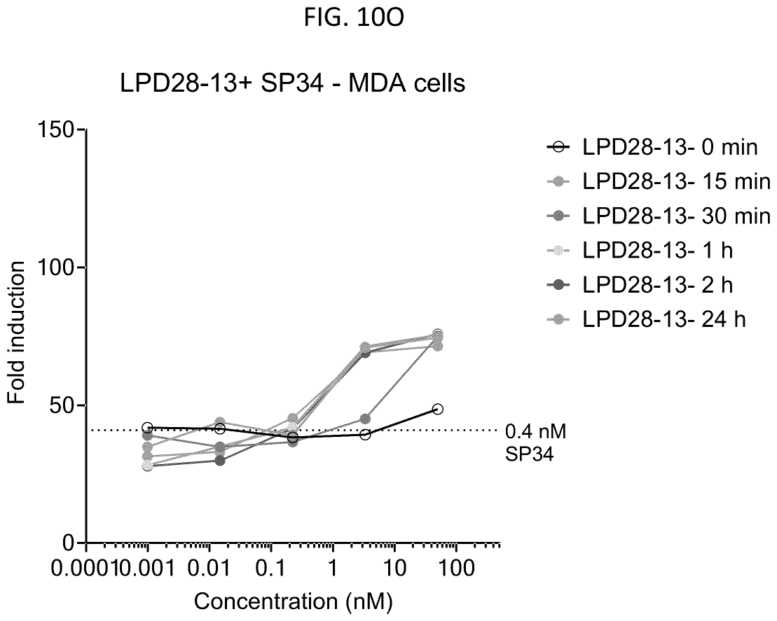

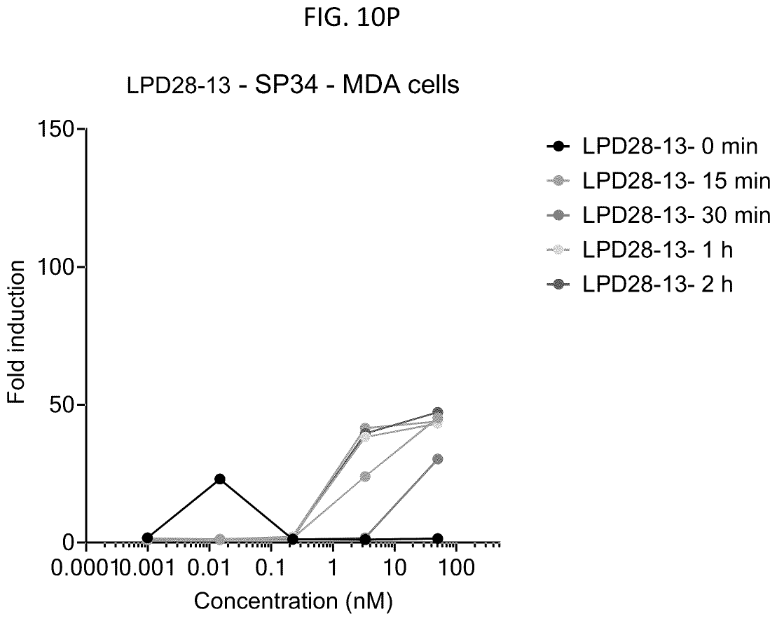

In contrast, two arm LPD molecules were able to elicit strong CD28 mediated luminescence responses dependent on the used condition, LHL linker combination and presence of specific CD28 V domain in the molecule. FIG. 10A shows that LPD28-10 achieved signals in the presence of CD3 stimulation and MDA-MB-231 cells. Importantly, intact/undigested and 8 h MMP12 treated LPD28-10 showed no signal over CD3 stimulation while highest signal of CD28 stimulation was achieved with LPD28-10 molecules treated for 15 min—this was accompanied by a marked hook effect as observed above for LPD28-1. 30 min MMP12 treatment resulted in slightly lower CD28 costimulation signal. 1 h and 2 h MMP12 treated LPD28-10 showed luminescence signals clearly above CD3 stimulation, confirming CD28 co-stimulation ability for 15 min, 30 min, 1 h and 2 h MMP12 treated LPD28-10. Absence of CD3 co-stimulation but presence of MDA-MB-231 cells showed no CD28 mediated luminescence signal for intact LPD28-10. In contrast, all MMP12 treated LPD28-10 molecules resulted in CD28 signal without the need for CD3 stimulation confirming the ability of TGN1412 derived CD28 V domains to elicit superagonistic signaling in two arm LPD protein formats. 15 min and 30 min digested LPD proteins again showed highest signals and a marked hook effect. 1 h, 2 h and 8 h treated LPD28-10 elicited similar luminescence signals suggesting a similar degree of digestion and removal of PDL1 binding domains from LPD28-10 (FIG. 10 B). This matches LPD28-10 digestion profiles in SDS-PAGE (FIG. 5F). Absence of MDA-MB-231 cells and therefore absence of PDL1 engagement resulted in overall higher fold inductions in Jurkat IL2 reporter cells speaking to the T cell suppressive nature of MDA-MB-231 cells. Again, intact and 8 h MMP12 digested LPD28-10 proteins did not show any stimulating activity. In contrast, all MMP12 digested LPD proteins showed similar luminescence signals with LPD28-10 treated for 15 min with MMP12 again resulting in highest signals (FIG. 10C). Interestingly, in the absence of PDL1 engagement (no MDA-MB-231 cells) and CD3 stimulation, all tested MMP12 treated LPD28-10 molecules stimulated Jurkat IL2 reporter cells to a similar degree (FIG. 10D). This contrasts 15 min and 30 min treated LPD28-10 fold induction signals shown in FIG. 10B in the presence of MDA-MB-231 cells. These results suggest the importance of synapse formation and simultaneous PDL1 V domain engagement to confer optimal (co)stimulatory activity.

In contrast to LPD28-10, LPD28-11 was generated containing CD28 V domains that are unable to produce superagonistic signals (FIG. 8). FIG. 10E shows that LPD28-11 was also able to achieve luminescence signals in the presence of CD3 stimulation and MDA-MB-231 cells. Intact LPD28-11 showed low signal over CD3 stimulation at the highest concentrations tested (50 nM) confirming ELISA and Jurkat cell binding results (FIG. 6D and FIG. 7C). Highest signal of CD28 stimulation was achieved with LPD28-11 molecules treated for 15 and 30 min—this was again accompanied by a marked hook effect as observed above for LPD28-1 and LPD28-10. 1 h and 2 h MMP12 treated LPD28-11 showed luminescence signals clearly above CD3 stimulation, confirming CD28 co-stimulation ability for 15 min, 30 min, 1 h and 2 h MMP12 treated LPD28-11. Absence of CD3 co-stimulation but presence of MDA-MB-231 cells showed no CD28 mediated luminescence signal for intact or MMP12 treated LPD28-11 as expected (FIG. 10F). Absence of MDA-MB-231 cells and therefore absence of PDL1 engagement again resulted in overall higher fold inductions in Jurkat IL2 reporter cells in the presence of CD3 stimulation as described above. Again, intact digested LPD28-11 showed stimulating activity at the highest concentration tested (FIG. 10G). In contrast, all MMP12 digested LPD proteins showed sustained stimulation activity across 3 of 5 tested concentrations with similar fold induction achieved across all MMP12 treatment times (FIG. 10G). As expected, in the absence of PDL1 engagement (no MDA-MB-231 cells) and CD3 stimulation, none of the tested intact or MMP12 treated LPD28-11 molecules stimulated Jurkat IL2 reporter cells (FIG. 10H).

Additional two arm LPD molecules, LPD28-12 and LPD28-13 both containing the TGN1412 derived CD28 binding V domains were tested in a similar fashion. LPD28-12 and -13 both contain LHL-FR linkers designed to confer faster MMP12 cleavage ability which was confirmed for LPD28-12 but not-13 in FIG. 5H. FIG. 10I shows that LPD28-12 achieved signals in the presence of CD3 stimulation and MDA-MB-231 cells. Importantly, intact LPD28-12 showed minimal signal over CD3 stimulation for the highest concentration tested. Strong signal of CD28 stimulation was achieved with LPD28-12 molecules treated for 15 min and 30 min, respectively—this was again accompanied by a marked hook effect as observed for previous LPD proteins. 1 h, 2 h and 24 h MMP12 treated LPD28-12 showed similar luminescence signals clearly above CD3 stimulation levels, confirming CD28 co-stimulation ability for 15 min, 30 min, 1 h, 2 h and 24 h MMP12 treated LPD28-12 (FIG. 10I). Absence of CD3 co-stimulation but presence of MDA-MB-231 cells showed no CD28 mediated luminescence signal for intact LPD28-12 (FIG. 10J). In contrast, all MMP12 treated LPD28-12 molecules resulted in CD28 signal without the need for CD3 stimulation confirming the ability of TGN1412 derived CD28 V domains to elicit superagonistic signaling in two arm LPD protein formats. The degree of LPD28-12 induced co-stimulation varies little between MMP12 treatment times, matching LPD28-12 digestion profiles in SDS-PAGE (FIG. 5H) which suggested full digestion of intact heavy chains at 15 min, with some remaining intact light chain remaining until 30 min digestion timepoints. Absence of MDA-MB-231 cells and therefore absence of PDL1 engagement resulted again in overall higher fold inductions in Jurkat IL2 reporter cells. Again, intact LPD28-12 protein showed some stimulating activity at its highest concentration tested (FIG. 10K). In contrast, all MMP12 digested LPD proteins showed similar luminescence signals with LPD28-12 treated for 15 min resulting in highest signals (FIG. 10K). As observed for LPD28-10, in the absence of PDL1 engagement (no MDA-MB-231 cells) and CD3 stimulation, all tested MMP12 treated LPD28-12 molecules stimulated Jurkat IL2 reporter cells to an almost indistinguishable degree (FIG. 10L).

Next, LPD28-13 was tested with Jurkat IL2 reporter cells as described. FIG. 10M shows that LPD28-13 achieved signals in the presence of CD3 stimulation and MDA-MB-231 cells. Importantly, intact LPD28-13 showed no signal over CD3 stimulation. Strong signal of CD28 stimulation was achieved with LPD28-13 molecules treated for 30 min and 1 h, respectively—this was accompanied by a marked hook effect as observed for previous LPD proteins. 15 min, 2 h and 24 h MMP12 treated LPD28-13 showed similar luminescence signals clearly above CD3 stimulation levels, confirming CD28 co-stimulation ability for 15 min, 30 min, 1 h, 2 h and 24 h MMP12 treated LPD28-12 (FIG. 10M) as before. Absence of CD3 co-stimulation but presence of MDA-MB-231 cells showed no CD28 mediated luminescence signal for intact LPD28-13 (FIG. 10N). In contrast, all MMP12 treated LPD28-13 molecules resulted in CD28 signal without the need for CD3 stimulation. Highest levels were achieved for 30 min digestion timepoint. All remaining MMP12 treated LPD28-13 proteins induced co-stimulation over background (FIG. 10N). Absence of MDA-MB-231 cells and therefore absence of PDL1 engagement resulted again in overall higher fold inductions in Jurkat IL2 reporter cells. Intact LPD28-13 protein showed some stimulating activity at its highest concentration tested (FIG. 10O). All MMP12 digested LPD proteins showed similar luminescence signals (FIG. 10O). As observed for LPD28-10, in the absence of PDL1 engagement (no MDA-MB-231 cells) and CD3 stimulation, all tested MMP12 treated LPD28-13 molecules stimulated Jurkat IL2 reporter cells to a very similar degree (FIG. 10L) compared to results shown in the presence of MDA-MB-231 cells (FIGS. 10M and N). Taken together, this data confirms the importance of synapse formation and simultaneous PDL1 V domain engagement for LPD proteins to confer optimal (co)stimulatory CD28 activity. This shows that CD28 V domains, their epitope, LPD linker designs as well as LPD protein modality strongly influence the capacity of LPD proteins to activate CD28.

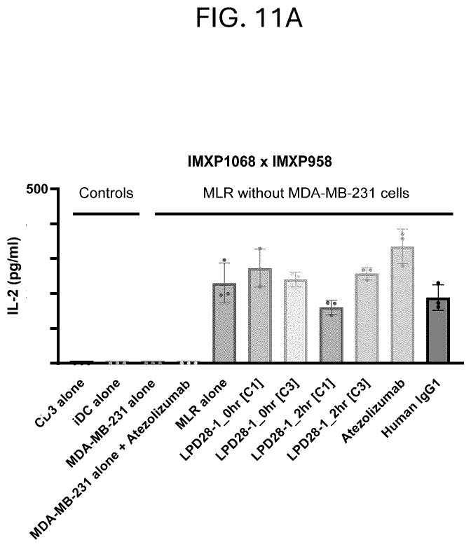

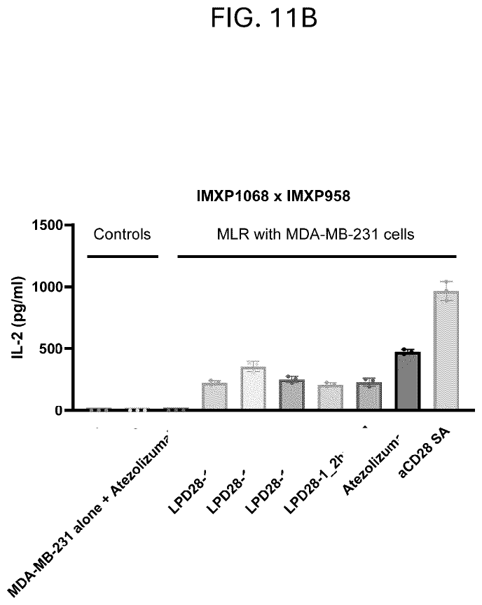

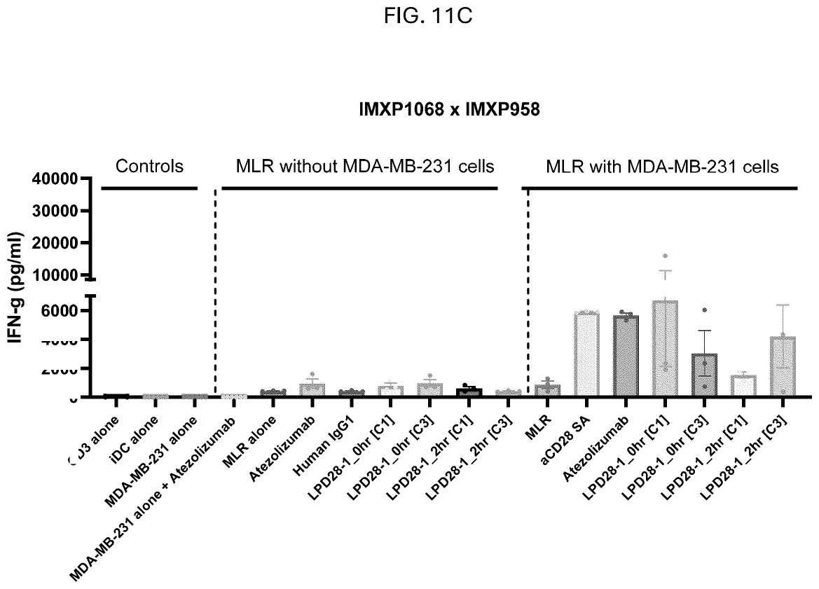

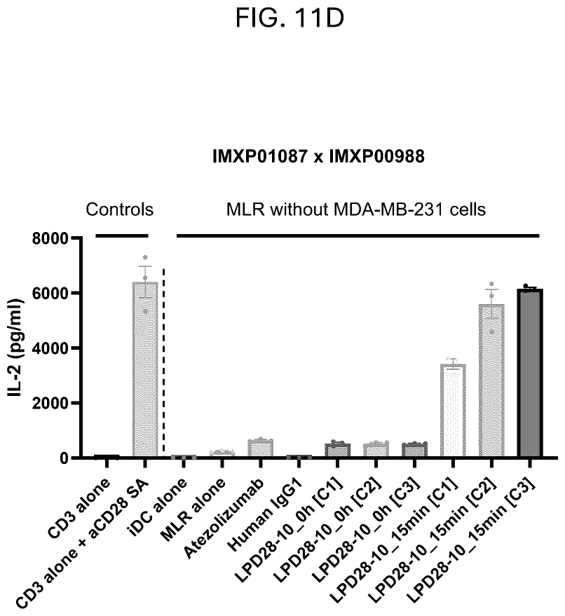

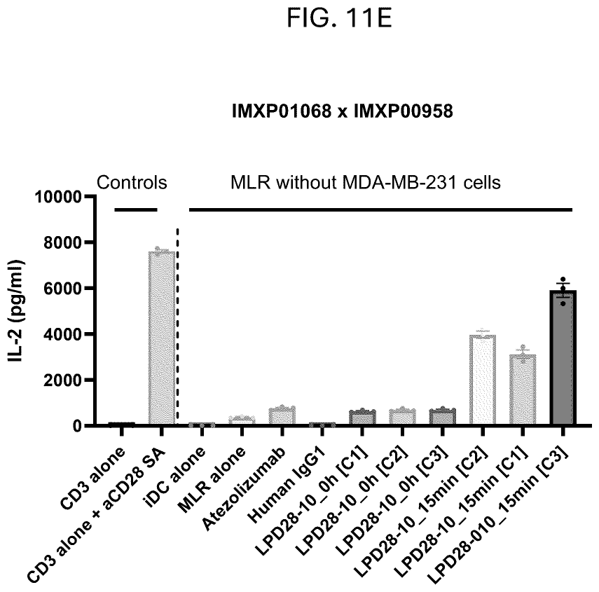

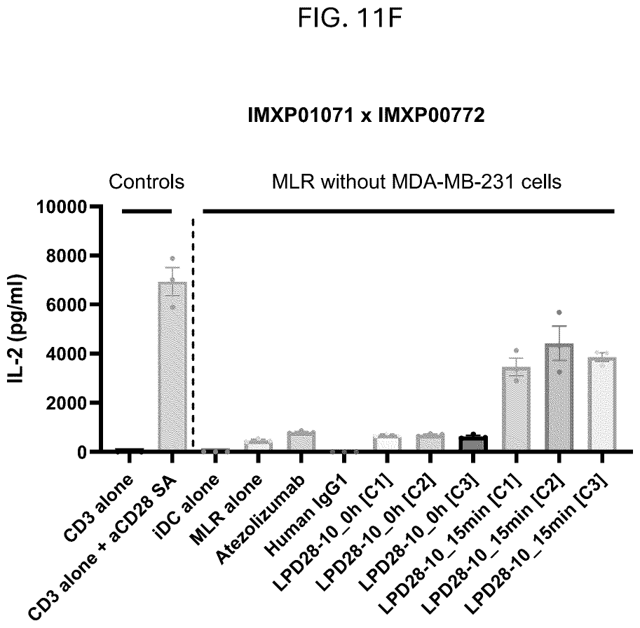

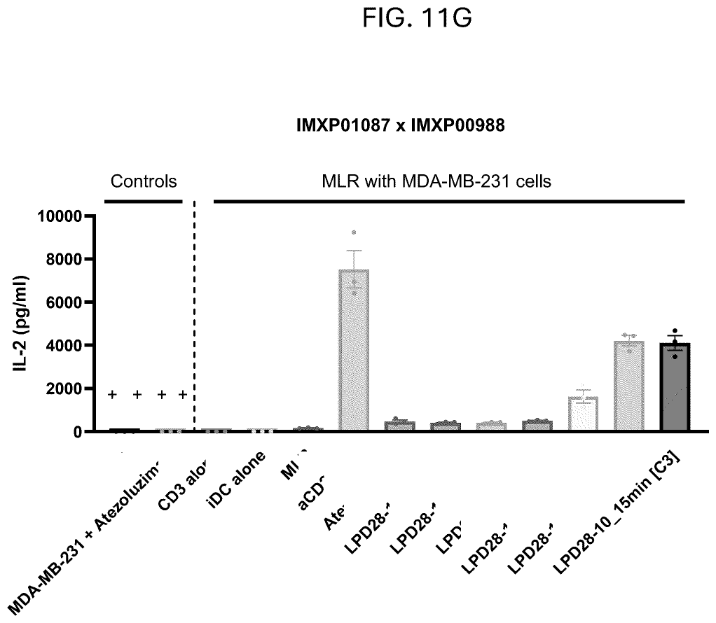

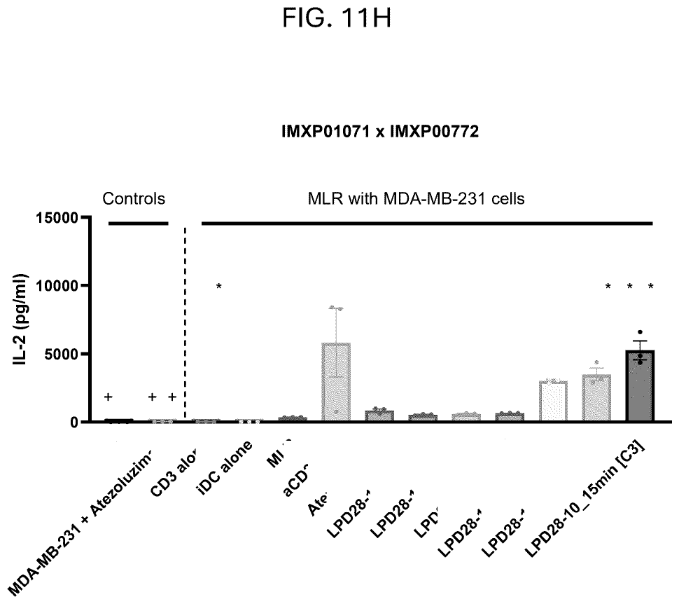

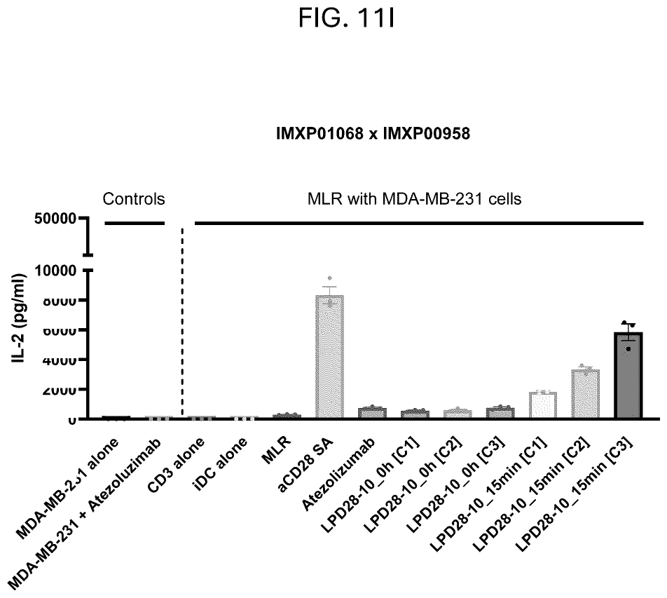

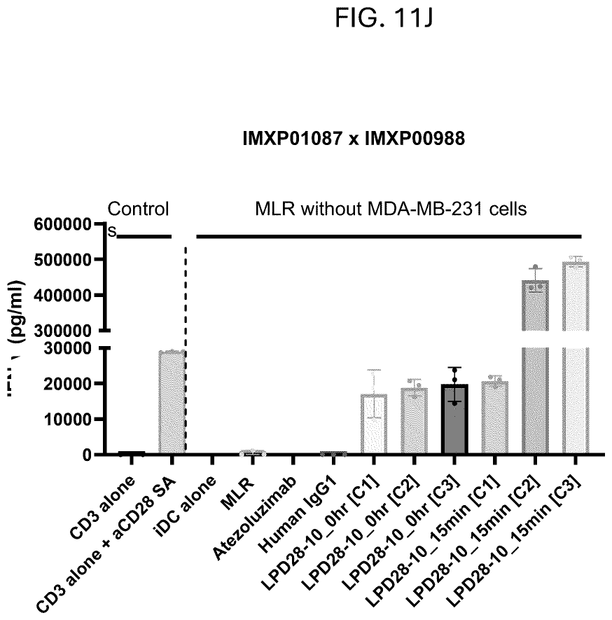

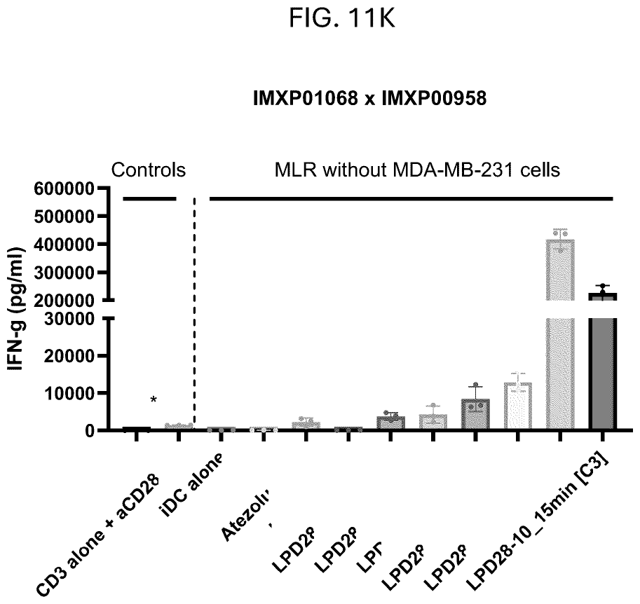

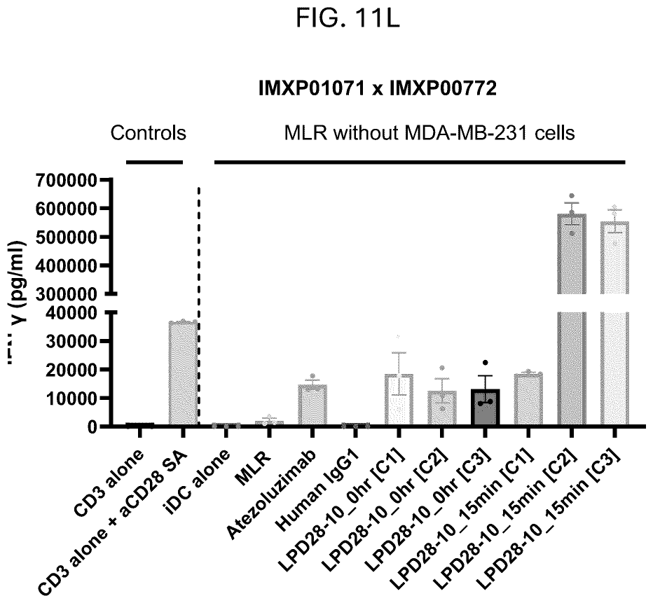

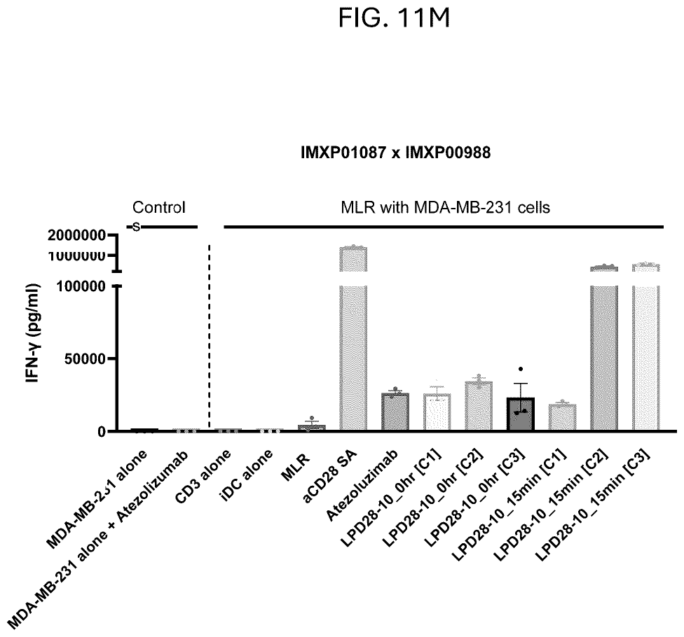

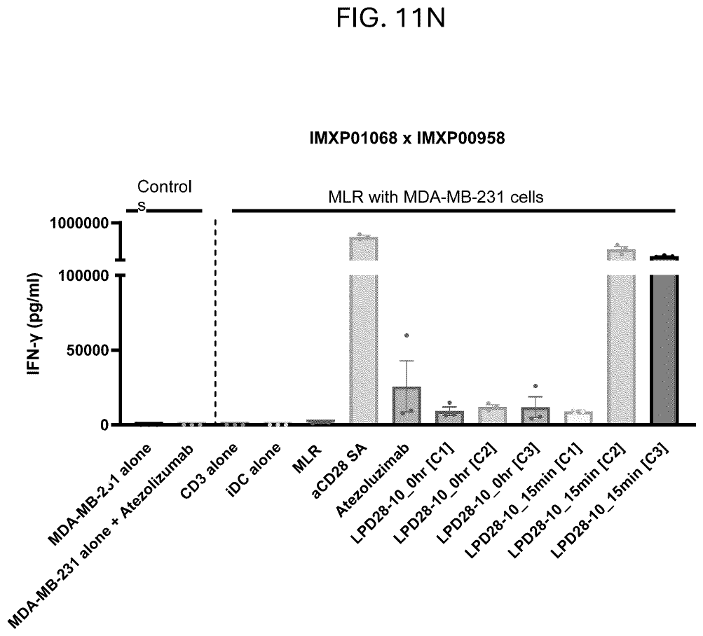

To further examine the influence of LPD proteins in stimulating immune activation, representative LPD28 proteins were tested in a mixed lymphocyte reaction assay. Briefly, monocytes isolated from peripheral blood mononuclear cells (PBMCs) from one donor were allowed to differentiate into immature dendritic cells (iDCs) and were hereafter co-cultured with CD3+ T cells from a non-matching donor. This is sought to result in a low level of CD3 activation signal of T cells. MDA-MB-231 cells were incubated with the mixed lymphocyte reaction in the presence or absence of intact or MMP12 treated LPD molecules. Test and/or control compounds were added and production of the cytokines interleukin-2 (IL-2) and interferon-γ (IFN-γ) was measured by ELISA after two and five days, respectively, to evaluate the immunomodulatory potential of selected LPD proteins. Intact and 2 h MMP12 treated LPD28-1 was incubated in the presence or absence of MDA-MB-231 cells in the mixed lymphocyte reaction at the indicated concentrations C1=20 nM. C2=6.6 nM and C3=2.2 nM (FIGS. 11A1 and 11A2 and B). Presence of IL-2 or IFNg in the supernatant was used as biomarker for T cell activity. Neither intact nor 2 h MMP12 treated LPD28-1 proteins resulted in significant IL2 or IFNg release over background MLR signal, in the presence or absence of MDA-MD-231 cells. 3 donor pairs were tested and the result of a representative donor pair is shown in FIG. 11A1 and FIGS. 11A2 and B. In contrast, LPD28-10 MMP12 treated molecules were able to induce strong T cell activity resulting in high levels of IL2 and IFNg secretion significantly over background MLR levels and atezolizumab (PDL1 blockade only) at all concentrations and donor combinations tested (C1=20 nM, C2=7 nM, and C3=2 nM) (FIGS. 11D-11I, FIGS. 11J-11L and FIGS. 11M-11O). Lowest LPD28-10 concentrations showed the highest cytokine secretion, again suggesting a hook effect as observed in Jurkat IL2 reporter cells. Importantly, intact LPD28-10 proteins did not induce cytokine secretion above control PDL1 block levels (atezolizumab control) (IL2: FIGS. 11C1-3 and FIGS. 11F-H; IFNg: FIGS. 11I-K and FIGS. 11L-N). This supports that LPD design and modality can strongly influence their ability to elicit cytokine release in primary cells.

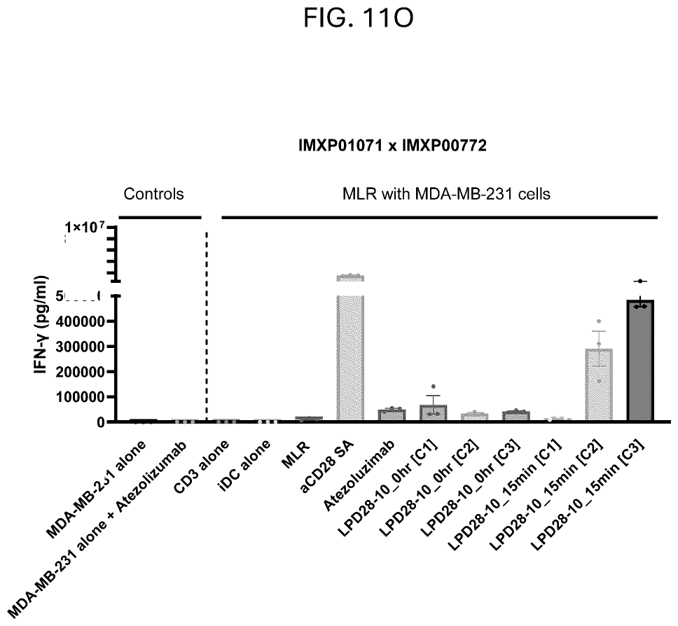

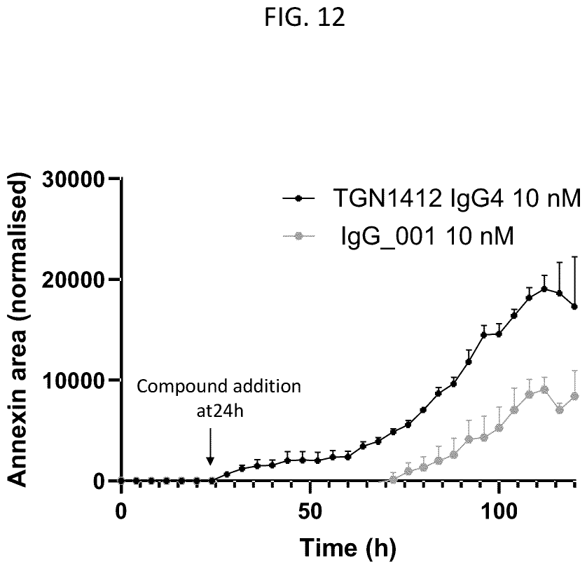

To examine the effect of PD-L1 blockade in combination with potent CD28 costimulation, selected LPD proteins were tested with primary T cells to evaluate their potential to mediate cytotoxic killing of MDA-MD-231 cells. This effect was measured via induction of annexin signal in the MDA-MB-231 cells (FIG. 12). First, baseline cytotoxicity over buffer conditions only were established with commercially available TGN1412 (IgG4) and TGN derived CD28 V domain IgG_001 on IgG1-Fc isotype (FIG. 12). Cytotoxicity levels are markedly over background within hours of TGN1412 (IgG4) addition and approximately 48 h post addition of compound for IgG_001, again confirming the tunable nature of superagonism and higher activity of superagonistic antibodies with IgG4 isotype.

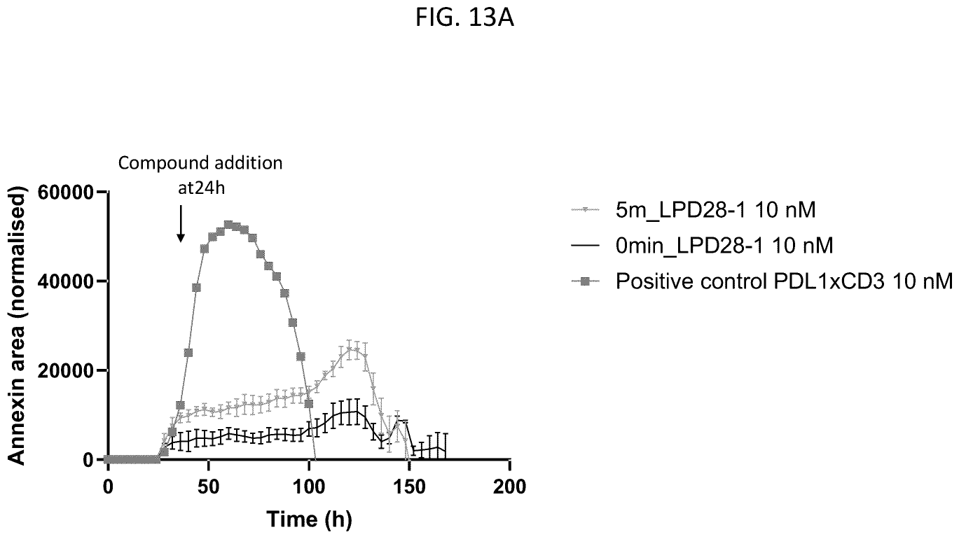

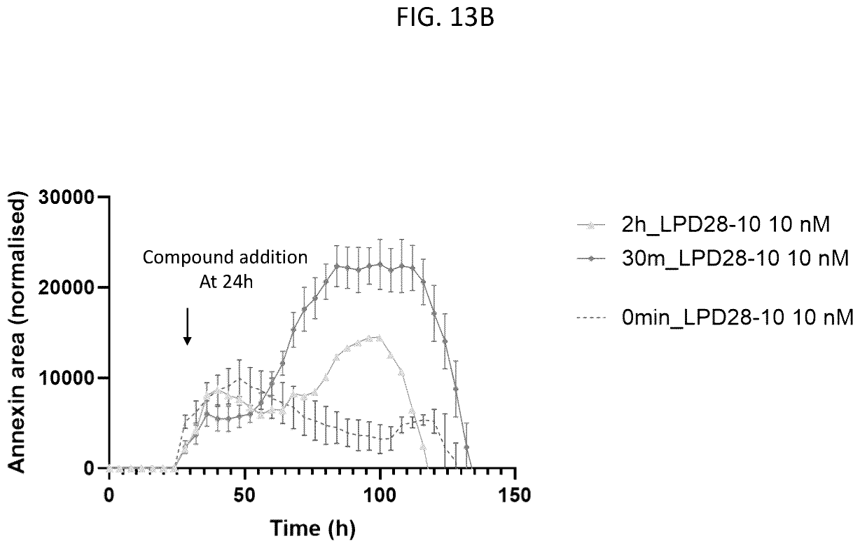

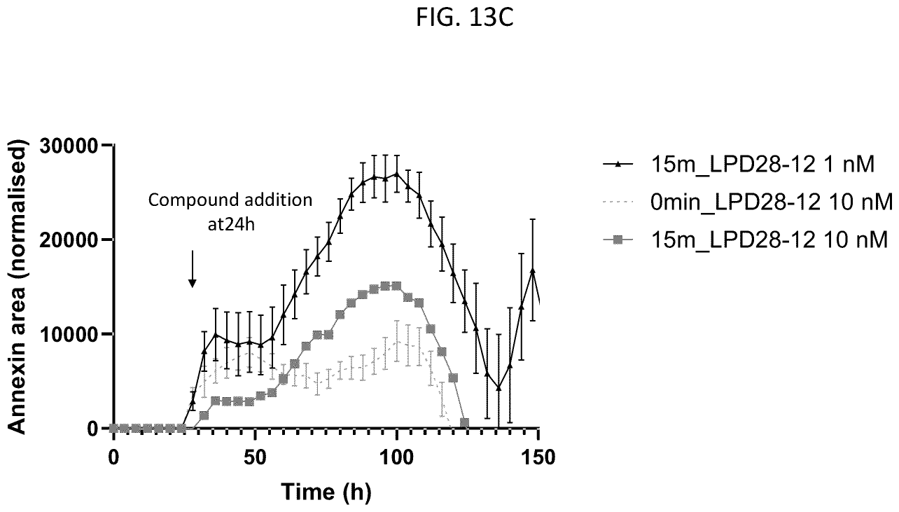

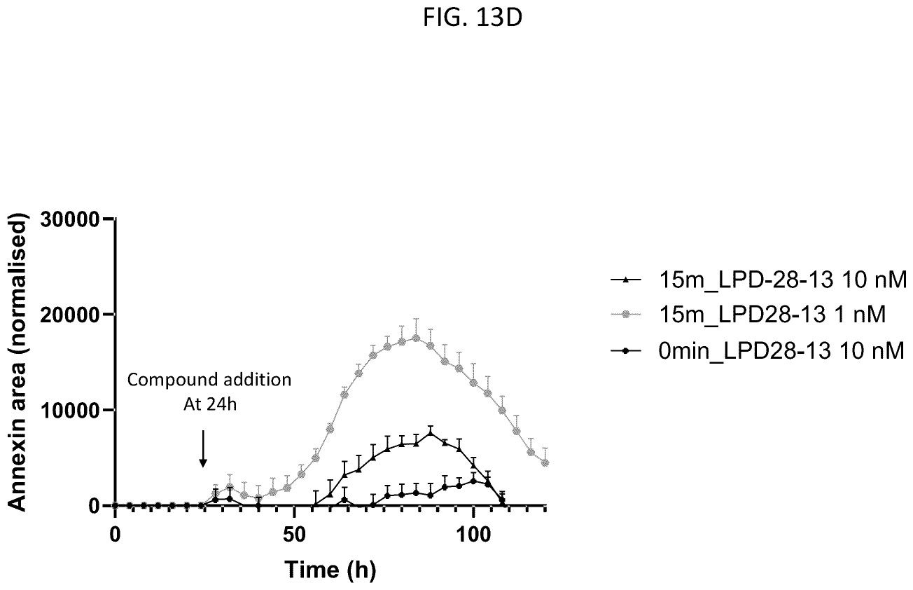

LPD28-1, -10 and -12 proteins were incubated on the same plate and in comparison, to a PDL1×CD3 engager as positive control. No CD3 co-stimulation was provided. Undigested LPD28-1 conferred low level of cytotoxicity over background, but much lower than PDL1×CD3 positive control. MMP12 treated (2 h) LPD28-1 showed an increase in cytotoxicity over undigested molecule which was again lower than positive control (FIG. 13A). This cytotoxic activity is surprising as single agent, CD28 mediated cytotoxicity without CD3 co-stimulation has not been described. It is interesting to note that cytokine secretion was undetectable in MLR assays for LPD28-1 (FIG. 11) which may speak to an interesting phenotype of this compound combining an ability to confer cytotoxicity but with low concomitant cytokine release. Similarly, intact LPD28-10, LPD28-12 and LPD28-13 showed low levels of cytotoxicity with a marked increase in annexin signals after MMP12 treatment at indicated times (FIGS. 13B, C and D). LPD28-10 molecules were tested after 30 min and 2 h MMP12 treatment. Interestingly, 2 h MMP12 treated LPD28-10 showed lower ability to induce annexin signals, a measure of cytotoxicity, than 30 min treated LPD28-10 (FIG. 13B). Longer MMP12 treatment results in higher degree of fully cleaved molecules and PDL1 Fab removal. This again suggests the need for simultaneous PDL-1 and CD28 engagement to optimally activate T cells and direct activity. In addition, LPD28-12 and LPD28-13 were titrated using 1 nM and 10 nM of MMP12 treated compounds (FIGS. 13C and D). This resulted in higher overall cytotoxicity at lower concentrations (1 nM)—again suggesting a hook effect with higher compound concentrations leading to impairment of activity in immune cells. In summary, combining PDL1 blockade with strong CD28 stimulation in LPD molecules conveys single agent cytotoxic activity without the need for CD3 costimulation.

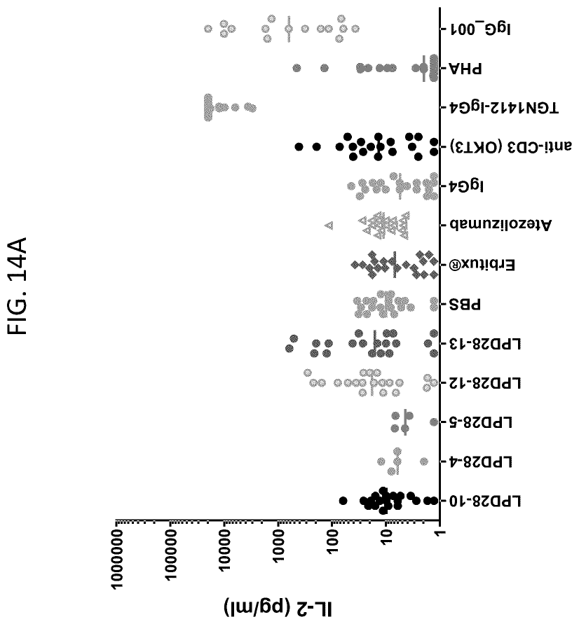

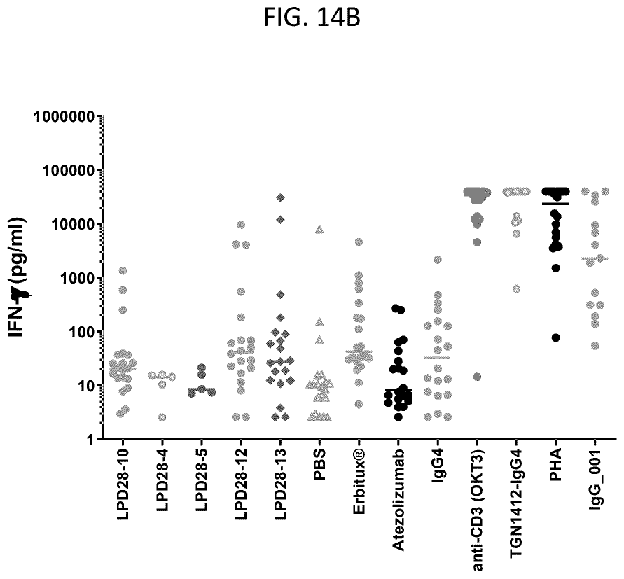

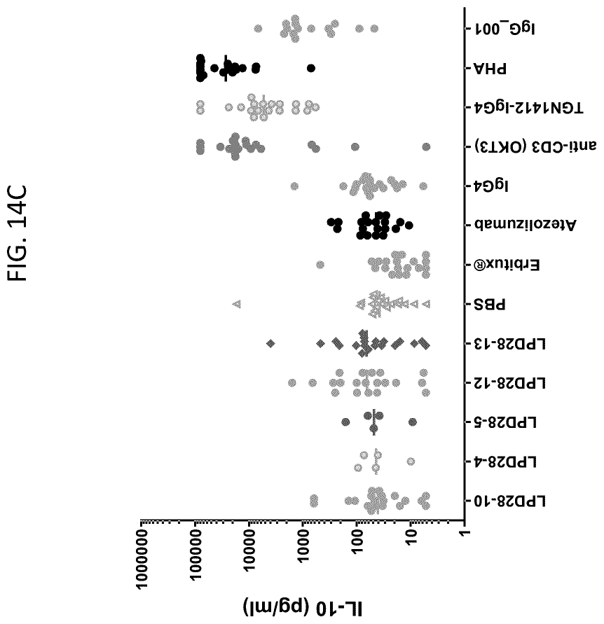

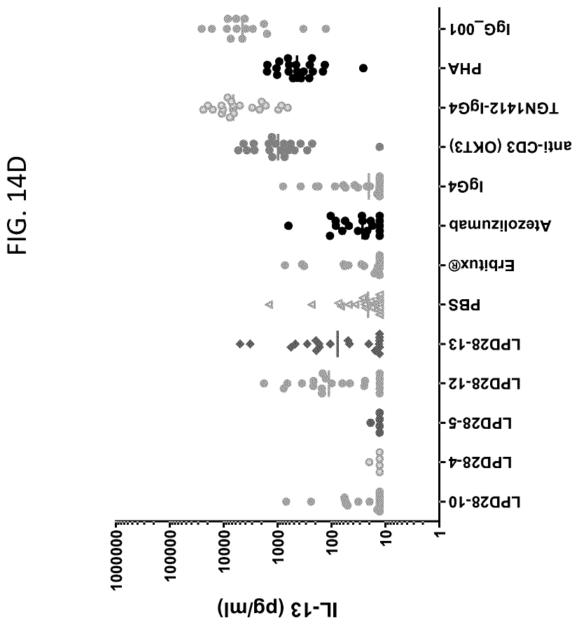

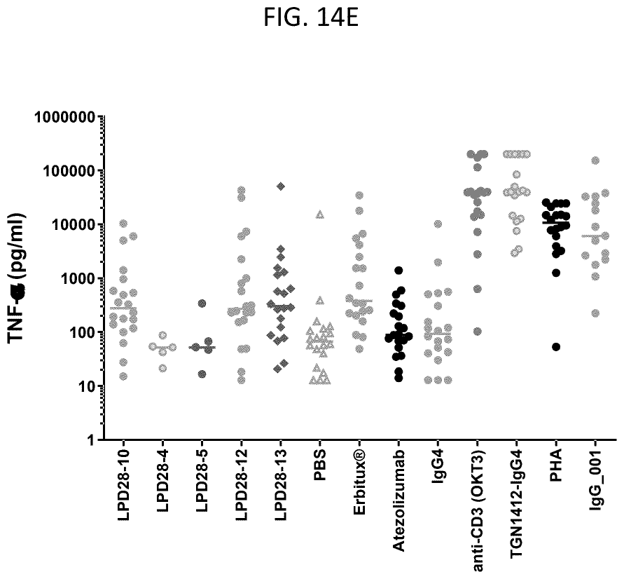

To examine the safety potential of selected and intact/undigested LPD compounds, they were subjected to cytokine release assay. Briefly, all proteins were coated onto 96 well plates at high density (1 ug/ml) and incubated with PBMC from 20 different healthy donors for 72 h hours. Supernatants were then harvested and tested for cytokine secretion (FIG. 13A-F). As expected, high levels of IL2 secretion were detected for TGN1412 (IgG4) and IgG_001 (TGN1412 V domain derived CD28 binder on IgG1 Fc backbone)—with the latter stimulating lower IL2 release. This again confirms the previously observed and expected quality of IgG4 vs IgG1 isotypes on CD28 superagonism. IL2 secretion levels for the majority of donors was uniformly high for TGN1412 (IgG4), resulting in up the 80000 pg/ml IL2—the upper limit of quantitation in the assay and en par with anti-CD3 (OKT3) positive control. IgG_001 elicited a mean of approximately 2000 pg/ml of IL2 secretion in the PBMC from twenty healthy donors tested. 2 arm LPD molecules LPD28-10, LPD28-12 and LPD28-13 did not result in mean IL2 secretion above 100 pg/ml, similar to mean levels reached in PBS, Erbitux and atezolizumab tested in the same donors as negative controls. This result confirmed that intact LPD molecules are effective at shielding CD28 V domains from interacting with its target on CD28+ T cells. Additional one arm LPD molecules, LPD 28-4 and LPD28-5, were also tested in this assay and showed similarly low levels of mean IL2 secretion <100 pg/ml (total of 5 donors tested) as described for 2 arm LPD molecules (FIG. 14A).

Next, IFNg secretion levels were detected for TGN1412 (IgG4) and IgG_001 (TGN1412 V domain derived CD28 binder on IgG1 Fc backbone) in the same supernatants—with the latter stimulating lower IFNg release again confirming the previously observed and expected quality of IgG4 vs IgG1 backbones on CD28 superagonism. IFNg secretion levels for the majority of donors was uniformly high for TGN1412 (IgG4), resulting in up the 20000 pg/ml IFNg—the upper limit of quantitation in the assay and en par with anti-CD3 (OKT3) positive control. IgG_001 elicited a mean of approximately 800 pg/ml of IFNg secretion in supernatants. Surprisingly, 2 arm LPD molecules LPD28-10, LPD28-12 and LPD28-13 did not result in mean IFNg secretion above 200 pg/ml, similar to mean levels reached in PBS, Erbitux and atezolizumab tested in the same donors as negative controls. Additional one arm LPD molecules, LPD 28-4 and LPD28-5, were also tested in this assay and showed similarly low levels of mean IFNg secretion <100 pg/ml (total of 5 donors tested) with these compounds and as described for 2 arm LPD molecules (FIG. 14B).

Next, IL-10 secretion levels were detected for TGN1412 (IgG4) and IgG_001 (TGN1412 V domain derived CD28 binder on IgG1 Fc backbone) in the same supernatants—with the latter stimulating lower IL-10 release again confirming the previously observed and expected quality of IgG4 vs IgG1 backbones on CD28 superagonism. IL-10 secretion levels for the majority of donors was uniformly high for TGN1412 (IgG4), resulting in mean levels of approximately 5000 pg/ml IL-10, lower compared to mean levels reached with anti-CD3 (OKT3) positive control (~20000 pg/ml). IgG_001 elicited a mean of approximately 2000 pg/ml of IL-10 secretion in supernatants. Surprisingly, 2 arm LPD molecules LPD28-10, LPD28-12 and LPD28-13 did not result in mean IL-10 secretion above 100 pg/ml, similar to mean levels reached in PBS, Erbitux and Atezolizumab tested in the same donors as negative controls. Additional one arm LPD molecules, LPD 28-4 and LPD28-5, were also tested in this assay and showed similarly low levels of mean IL-10 secretion <100 pg/ml (total of 5 donors tested) with these compounds and as described for 2 arm LPD molecules (FIG. 14C).

Next, IL-13 secretion levels were detected for TGN1412 (IgG4) and IgG_001 (TGN1412 V domain derived CD28 binder on IgG1 Fc backbone) in the same supernatants—with the latter stimulating lower IL-13 release again confirming the previously observed and expected quality of IgG4 vs IgG1 backbones on CD28 superagonism. IL-13 secretion level for the majority of donors was uniformly high for TGN1412 (IgG4), resulting in mean levels of approximately 8000 pg/ml IL-13, significantly higher compared to mean levels reached with anti-CD3 (OKT3) positive control (~1000 pg/ml). IgG_001 elicited a mean of approximately 5000 pg/ml of IL-13 secretion in supernatants. Surprisingly, 2 arm LPD molecule LPD28-10 did not reach mean IL-13 levels above 100 pg/ml, similar to approximate mean levels reached in PBS, Erbitux and atezolizumab tested in the same donors as negative controls. LPD28-12 and LPD28-13 reached mean levels of approximately 100 pg/ml. Additional one arm LPD molecules, LPD 28-4 and LPD28-5, were also tested in this assay and showed similarly low levels of mean IL-10 secretion <100 pg/ml (total of 5 donors tested) with these compounds and as described for 2 arm LPD molecules (FIG. 14D).

Next, TNF alpha secretion levels were detected for TGN1412 (IgG4) and IgG_001 (TGN1412 V domain derived CD28 binder on IgG1 Fc isotype) in the same supernatants—with the latter stimulating lower TNFalpha release again confirming the previously observed and expected quality of IgG4 vs IgG1 backbones on CD28 superagonism. TNFalpha secretion level for the majority of donors was uniformly high for TGN1412 (IgG4), resulting in mean levels of approximately 30000 pg/ml TNFalpha-en par with mean levels reached with anti-CD3 (OKT3) positive control. IgG_001 elicited a mean of approximately 10000 pg/ml of TNFalpha secretion in supernatants. Surprisingly and interestingly, 2 arm LPD molecules LPD28-10, LPD28-12 and LPD28-13 did not result in mean TNFalpha secretion above 350 pg/ml, similar to mean levels reached in PBS, Erbitux and atezolizumab tested in the same donors as negative controls. Additional one arm LPD molecules, LPD 28-4 and LPD28-5, were also tested in this assay and showed similarly low levels of mean TNFalpha secretion <100 pg/ml (total of 5 donors tested) with these compounds and as described for two arm LPD molecules (FIG. 14E).

Finally, IL-6 secretion levels were detected for TGN1412 (IgG4) and IgG_001 (TGN1412 V domain derived CD28 binder on IgG1 Fc isotype) in the same supernatants—with the latter stimulating lower IL-6 release again confirming the previously observed and expected quality of IgG4 vs IgG1 backbones on CD28 superagonism. IL-6 secretion level for the majority of donors was uniformly high for TGN1412 (IgG4), resulting in mean levels of approximately 20000 pg/ml IL-6—the upper end of LOQ in this assay-en par with mean levels reached with anti-CD3 (OKT3) positive control. IgG_001 elicited a mean of approximately 300 pg/ml of IL-6 secretion in supernatants. 2 arm LPD molecules LPD28-10, LPD28-12 and LPD28-13 did not result in mean IL-6 secretion above 200 pg/ml, similar to mean levels reached in PBS, Erbitux and atezolizumab tested in the same donors as negative controls. Additional one arm LPD molecules, LPD 28-4 and LPD28-5, were also tested in this assay and showed similarly low levels of mean IL-6 secretion <100 pg/ml (total of 5 donors tested) with these compounds and as described for 2 arm LPD molecules (FIG. 14F). LPD molecules show strongly increased safety potential compared to unmasked CD28 activating agents.

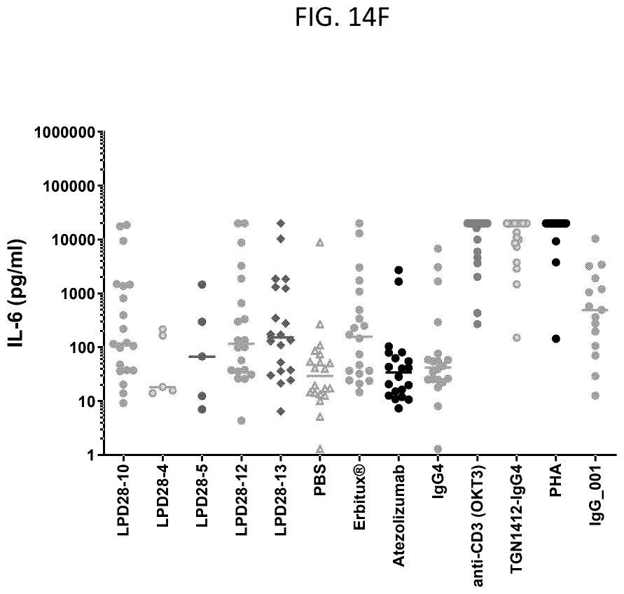

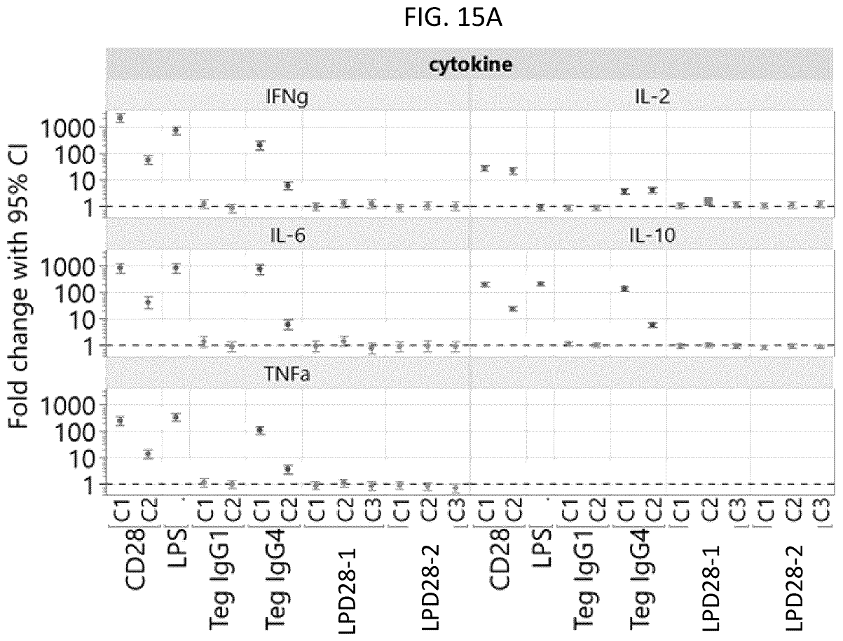

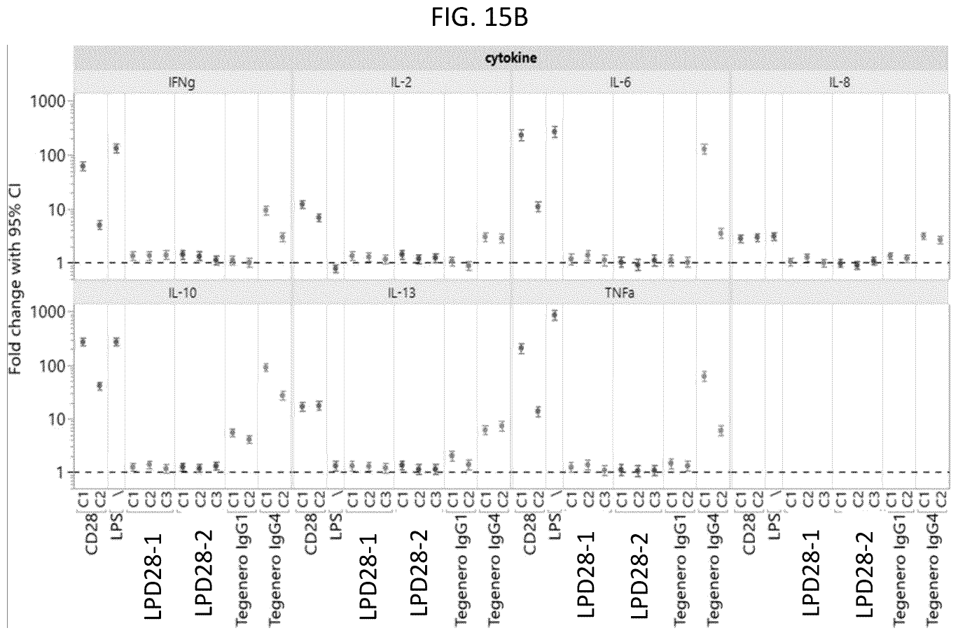

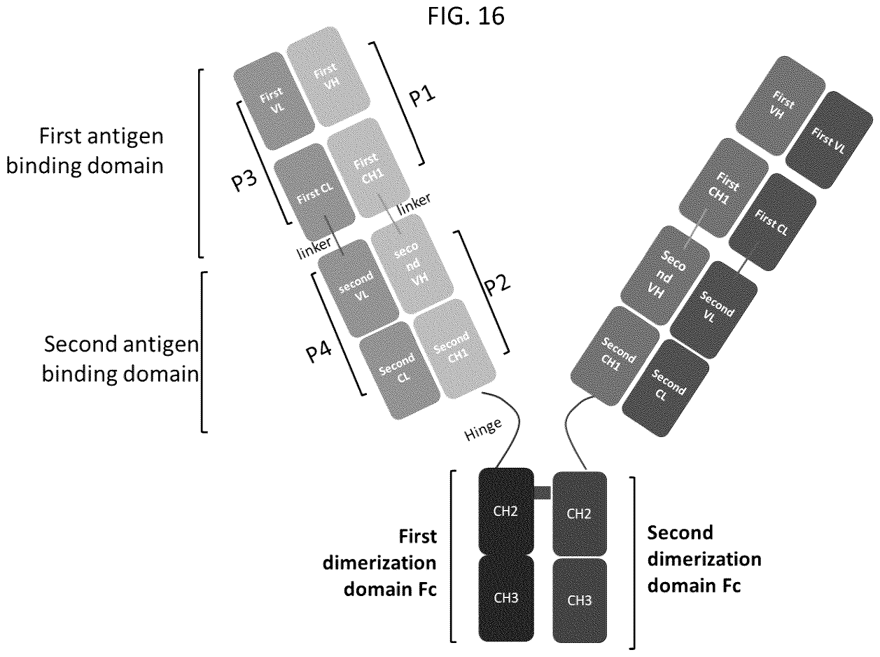

Next, to examine the safety potential of selected and intact/undigested LPD compounds, we sought to test selected LPD molecules in two additional cytokine release assays. Briefly, soluble and intact LPD28-1 and LPD28-2 molecules (C1=15 μg/mL, C2=2 μg/mL and C3=1 μg/mL) were incubated with PBMC at indicated concentrations isolated from 10 healthy donors. Supernatants were tested for cytokine secretion after 24 h (FIG. 15A). Mean fold changes over buffer control are shown in FIG. 15A. Interestingly, TGN1412 IgG4 was able to elicit strong fold changes in cytokine release for cytokines tested (IL-2, IL-6, IL-8, IL-10, IL-13, IFN-γ and TNF-α) however, IgG_001 was not able to produce changes in cytokine secretion to similar levels. Importantly, neither LPB28-1 and LPB28-2 showed significant fold changes over background for any of the tested cytokines.

Next, compounds were tested with a third PBMC cytokine release assay, termed RESTORE assay. This assay was first described and tested by Romer et al, Immunobiology 2011 in an attempt to establish a reliable PBMC assay for superagonism detection. Briefly, PBMCs were isolated from 10 healthy donors as before. However, contrasting classical PBMC cytokine release assays, the PBMCs are incubated for 48 h at 37 C. Following this incubation period, soluble control compounds and intact LPD28-1 and LPD28-2 molecules (C1=15 μg/mL, C2=2 μg/mL and C3=1 μg/mL) were mixed with 48 h rested PBMC. Supernatants were then tested for cytokine secretion after 24 h (FIG. 15B) as before. Mean fold changes over buffer control are shown in FIG. 15B. As previously, TGN1412 IgG4 was able to elicit strong cytokine release for cytokines tested: IL-2, IL-6, IL-8, IL-10, IL-13, IFN-γ and TNF-α, however, this time also IgG_001 was able to produce significant changes in cytokine secretion, confirming the increased sensitivity of this assay to compounds with CD28 superagonistic characteristics. Importantly, neither LPB28-1 and LPB28-2 showed significant fold changes over background for any of the tested cytokines in the RESTORE assay as well. This confirms that intact LPD28-1 and LPD28-2 are not able to stimulate cytokine secretion in a PBMC safety assay.

### Embodiments

1. A protein comprising a first polypeptide chain comprising a heavy chain and a second polypeptide chain comprising a light chain,
   wherein the heavy chain comprises, in N-terminus to C-terminus order, an anti-PD-L1 heavy chain variable (VH) domain, a first CH1 domain, a first linker, an anti-CD28 VH domain, and a second CH1 domain;
   wherein the light chain comprises, in N-terminus to C-terminus order, an anti-PD-L1 light chain variable (VL) domain, a first immunoglobulin light chain constant region, a second linker, an anti-CD28 VL domain, and a second immunoglobulin light chain constant region.
2. The protein of embodiment 1, wherein the heavy chain comprises in N-terminus to C-terminus order, the anti-PD-L1 VH domain, the first CH1 domain, the first linker, the anti-CD28 VH domain, the second CH1 domain, a hinge, a CH2 domain, and a CH3 domain.
3. The protein of embodiment 1 or 2, wherein the protein further comprises a third polypeptide chain comprising a hinge and a Fc region.
4. The protein of embodiment 3, wherein the third polypeptide chain comprises the amino acid sequence of SEQ ID NO: 32.
5. The protein of any one of embodiments 1-4, wherein the first linker comprises the amino acid sequence of SEQ ID NO: 1, SEQ ID NO: 2, SEQ ID NO: 3, or SEQ ID NO: 4.
6. The protein of any one of embodiments 1-5, wherein the second linker comprises the amino acid sequence of SEQ ID NO: 1, SEQ ID NO: 2, SEQ ID NO: 3, or SEQ ID NO: 4.
7. The protein of any one of embodiments 1-6,
   wherein the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14;
   wherein the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17;
   wherein the anti-CD28 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20;
   wherein the anti-CD28 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.
8. The protein of any one of embodiments 1-7, wherein the anti-PD-L1 VH domain comprises the amino acid sequence of SEQ ID NO: 24, and the anti-PD-L1 VL domain comprises the amino acid sequence of SEQ ID NO: 25.
9. The protein of any one of embodiments 1-8, wherein the anti-CD28 VH domain comprises the amino acid sequence of SEQ ID NO: 26.
10. The protein of any one of embodiments 1-9, wherein the anti-CD28 VL domain comprises the amino acid sequence of SEQ ID NO: 27.
11. The protein of any one of embodiments 1-10, wherein the heavy chain comprises the amino acid sequence of SEQ ID NO: 30 or SEQ ID NO: 31.
12. The protein of any one of embodiments 1-11, wherein the light chain comprises the amino acid sequence of SEQ ID NO: 28 or SEQ ID NO: 29.
13. The protein of embodiment 3, wherein
   (a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
   (b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.
14. The protein of embodiment 2 or 3, wherein the heavy chain comprises an IgG, IgE, IgM, IgD, IgA, or IgY constant region.
15. The protein of embodiment 2 or 3, wherein the heavy chain comprises an IgG1, IgG2, IgG3, IgG4, IgA1 or IgA2 constant region.
16. The protein of embodiment 2 or 3, wherein the heavy chain comprises an immunologically inert constant region.
17. The protein of embodiment 2 or 3, wherein the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kabat.
18. An immunoconjugate comprising the protein of any one of embodiments 1-17, linked to a therapeutic agent.
19. The immunoconjugate of embodiment 18, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.
20. A pharmaceutical composition comprising the protein of any one of embodiments 1-17 or the immunoconjugate of embodiment 18 or 19, and a pharmaceutically acceptable carrier, diluent or excipient.
21. A nucleic acid molecule encoding
   (a) the heavy chain amino acid sequence;
   (b) the light chain amino acid sequence; or
   (c) both the heavy chain and the light chain amino acid sequences of the protein of any one of embodiments 1-17.
22. An expression vector comprising the nucleic acid molecule of embodiment 21.
23. A recombinant host cell comprising the nucleic acid molecule of embodiment 21 or the expression vector of embodiment 22.
24. A method of producing a protein, the method comprising:
culturing a recombinant host cell comprising the expression vector of embodiment 22 under conditions whereby the nucleic acid molecule is expressed, thereby producing the protein; and
isolating the protein from the host cell or culture.
25. A method for enhancing an anti-cancer immune response in a subject, the method comprising administering to the subject a therapeutically effective amount of the protein of any one of embodiments 1-17, the immunoconjugate of embodiment 18 or 19, or the pharmaceutical composition of embodiment 20.
26. A method for treating cancer in a subject, the method comprising administering to the subject a therapeutically effective amount of the protein of any one of embodiments 1-17, the immunoconjugate of embodiment 18 or 19, or the pharmaceutical composition of embodiment 20.
27. A method for ameliorating a symptom of cancer in a subject, the method comprising administering to the subject a therapeutically effective amount of the protein of any one of embodiments 1-17, the immunoconjugate of embodiment 18 or 19, or the pharmaceutical composition of embodiment 20.
28. The method of any one of embodiments 25-27, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.
29. A protein comprising a first heavy chain and a first light chain, wherein the first heavy chain comprises:
   (a) a first portion of a first antigen binding domain (P1), comprising a first heavy chain variable domain (VH), and optionally a first heavy chain constant domain 1 (CH1);
   (b) a first linker, wherein the first linker is cleavable by a protease; and
   (c) a first portion of a second antigen binding domain (P2), comprising a second VH and optionally a second CH1; wherein:
      (i) the N-terminus of the first linker is linked to the C-terminus of the first CH1 of P1 and the C-terminus of the first linker is linked to the N-terminus of the second VH of P2; or
      (ii) the N-terminus of the first linker is linked to the C-terminus of the first VH of P1 and the C-terminus of the first linker is linked to the N-terminus of the second VH of P2; and
wherein the first light chain comprises:
   (a) a second portion of the first antigen binding domain (P3), comprising a first light chain variable domain (VL), and optionally a first light chain constant domain (CL);
   (b) a second linker, wherein the second linker is cleavable by a protease; and
   (d) a second portion of the second antigen binding domain (P4), comprising a second VL, and optionally a second CL; wherein:
      (i) the N-terminus of the second linker is linked to the C-terminus of the first CL of P3 and the C-terminus of the second linker is linked to the N-terminus of the second VL of P4; or
      (ii) the N-terminus of the first linker is linked to the C-terminus of the first VL of P3 and the C-terminus of the second linker is linked to the N-terminus of the second VL of P4; and
   wherein the first antigen binding domain binds to PD-L1; and
   wherein the when the first linker and the second linker are cleaved, the second antigen binding domain binds to CD28.
30. The protein of embodiment 29, wherein the CH1 of P1 is covalently linked to the CL of P3.
31. The protein of embodiment 29 or 30, wherein the CH1 of P2 is covalently linked to the CL of P4.
32. A protein comprising a first heavy chain and a first light chain,
wherein the first heavy chain comprises:
   (a) a first portion of a first antigen binding domain (P1), comprising a first heavy chain variable domain (VH);
   (b) a first linker, wherein the first linker is cleavable by a protease; and
   (c) a first portion of a second antigen binding domain (P2), comprising a second VH;
wherein the N-terminus of the first linker is linked to the C-terminus of P1, and the C-terminus of the first linker is linked to the N-terminus of the second VH of P2;
wherein the first light chain comprises:
   (c) a second portion of the first antigen binding domain (P3), comprising a first light chain variable domain (VL);
   (d) a second linker, wherein the second linker is cleavable by a protease; and
   (d) a second portion of the second antigen binding domain (P4), comprising a second VL;
wherein the N-terminus of the second linker is linked to the C-terminus of P3, and the C-terminus of the second linker is linked to the N-terminus of the second VL of P4;
wherein the first antigen binding domain binds to PD-L1; and
wherein the when the first linker and the second linker are cleaved, the second antigen binding domain binds to CD28.
33. The protein of embodiment 32, wherein the P1 further comprises a first heavy chain constant domain 1 (CH1), wherein the C-terminus of the first CH1 of P1 is linked to the N-terminus of the first linker.
34. The protein of embodiment 33, wherein the N-terminus of the first CH1 of P1 is linked to the C-terminus of the VH of P1.
35. The protein of embodiment 32, wherein the P3 further comprises a first CL, wherein the C-terminus of the first CL of P3 is linked to the N-terminus of the second liker.
36. The protein of embodiment 35, wherein the N-terminus of the first CL of P3 is linked to the C-terminus of the first VL of P3.
37. The protein of any one of embodiments 32-36, wherein the P2 further comprises a second CH1, wherein the N-terminus of the second CH1 of P2 is linked to the C-terminus of the second VH of P2.
38. The protein of any one of embodiments 32-37, wherein the P4 further comprises a second CL, wherein the N-terminus of the second CL of P4 is linked to the C-terminus of the second VL of P4.
39. The protein of any one of embodiments 35-38, wherein the first CH1 of P1 is covalently linked to the first CL of P3.
40. The protein of any one of embodiments 35-39, wherein the second CH1 of P2 is covalently linked to the second CL of P4.
41. The protein of any one of embodiments 29-40, wherein the first antigen binding domain comprises P1 and P3.
42. The protein of any one of embodiments 29-41, wherein the P1 and the P3 are covalently linked to form a Fab.
43. The protein of any one of embodiments 29-42, wherein the second antigen binding domain comprises P2 and P4.
44. The protein of any one of embodiments 29-43, wherein the P2 and the P4 are covalently linked to form a Fab.
45. The protein of any one of embodiments 29-44, wherein the first heavy chain further comprises a first dimerization domain.
46. The protein of embodiment 45, wherein the first dimerization domain comprises a first hinge domain and a first immunoglobulin constant domain (Fc domain).
47. The protein of embodiment 46, wherein the Fc domain comprises a first heavy chain constant domain 2 (CH₂) and a first heavy chain constant domain 3 (CH3).
48. The protein of embodiments 45-47, wherein the C-terminus of the P2 is linked to the N-terminus of the first hinge domain.
49. The protein of any one of embodiments 29-48, wherein the first linker comprises an amino acid sequence having at least 85% identity to any one of amino acid sequence of SEQ ID NO: 1, SEQ ID NO: 2, SEQ ID NO: 3, SEQ ID NO: 4, or SEQ ID NO: 33.
50. The protein of embodiment 49, wherein the first linker comprises an amino acid sequence of SEQ ID NO: 1, SEQ ID NO: 2, SEQ ID NO: 3, SEQ ID NO: 4, or SEQ ID NO: 33.
51. The protein of any one of embodiments 29-50, wherein the second linker comprises an amino acid sequence having at least 85% identity to any one of amino acid sequence of SEQ ID NO: 1, SEQ ID NO: 2, SEQ ID NO: 3, SEQ ID NO: 4, or SEQ ID NO: 33.
52. The protein of embodiment 51, wherein the second linker comprises an amino acid sequence of SEQ ID NO: 1, SEQ ID NO: 2, SEQ ID NO: 3, SEQ ID NO: 4, or SEQ ID NO: 33.
53. The protein of any one of embodiments 29-52, wherein the first VH of P1 comprises:
   (a) a heavy chain complementarity determining region 1 (HCDR1) amino acid sequence of SEQ ID NO: 12, SEQ ID NO: 34, or SEQ ID NO: 36;
   (b) a heavy chain complementarity determining region 2 (HCDR2) amino acid sequence of SEQ ID NO: 13, SEQ ID NO: 35, or SEQ ID NO: 37; and
   (c) and a heavy chain complementarity determining region 3 (HCDR3) amino acid sequence of SEQ ID NO: 14 or SEQ ID NO: 38.
54. The protein of any one of embodiments 29-53, wherein the first VL of P3 comprises:
   (a) a light chain complementarity determining region 1 (LCDR1) amino acid sequence of SEQ ID NO: 15 or SEQ ID NO: 39;
   (b) a light chain variable domain comprising a light chain complementarity determining region 1 (LCDR2) amino acid sequence of SEQ ID NO: 16 or DA; and
   (c) a light chain variable domain comprising a light chain complementarity determining region 3 (LCDR3) amino acid sequence of SEQ ID NO: 17.
55. The protein of any one of embodiments 29-54, wherein the second VH of P2 comprises:
   (a) a HCDR1 amino acid sequence of SEQ ID NO: 40, SEQ ID NO: 43, or SEQ ID NO: 45;
   (b) a HCDR2 amino acid sequence of SEQ ID NO: 41, SEQ ID NO: 44, or SEQ ID NO: 46; and
   (c) a HCDR3 amino acid sequence of SEQ ID NO: 42 or SEQ ID NO: 47.
56. The protein of any one of embodiments 29-55, wherein the second VL of P4 comprises:
   (a) a LCDR1 amino acid sequence of SEQ ID NO: 48 or SEQ ID NO: 51;
   (b) a LCDR2 amino acid sequence of SEQ ID NO: 49 or KA; and
   (c) a LCDR3 amino acid sequence of SEQ ID NO: 50.
57. The protein of any one of embodiments 29-54, wherein the second VH of P2 comprises:
   (a) a HCDR1 amino acid sequence of SEQ ID NO: 53, SEQ ID NO: 56, or SEQ ID NO: 58;
   (b) a HCDR2 amino acid sequence of SEQ ID NO: 54, SEQ ID NO: 57, or SEQ ID NO: 59; and
   (c) a HCDR3 amino acid sequence of SEQ ID NO: 55 or SEQ ID NO: 60.
58. The protein of any one of embodiments 29-54 and 57, wherein the second VL of P4 comprises:
   (a) a LCDR1 amino acid sequence of SEQ ID NO: 61 or SEQ ID NO: 64;
   (b) a LCDR2 amino acid sequence of SEQ ID NO: 62 or AA; and
   (c) a LCDR3 amino acid sequence of SEQ ID NO: 63.
59. The protein of any one of embodiments 29-58, wherein the first VH of P1 comprises a sequence having at least 75%, 80%, 85%, 90%, 95%, or 99% sequence identity to the sequence of SEQ ID NO: 24.
60. The protein of any one of embodiments 29-59, wherein the first VH of P1 comprises the sequence of SEQ ID NO: 24.
61. The protein of any one of embodiments 29-60, wherein the first VL of P3 comprises a sequence having at least 75%, 80%, 85%, 90%, 95%, or 99% sequence identity to the sequence of SEQ ID NO: 25.
62. The protein of any one of embodiments 29-61, wherein the first VL of P3 comprises a sequence of SEQ ID NO: 25.
63. The protein of any one of embodiments 29-56 and 59-62, wherein the second VH of P2 comprises a sequence having at least 75%, 80%, 85%, 90%, 95%, or 99% sequence identity to the sequence of SEQ ID NO: 26.
64. The protein of any one of embodiments 29-56 and 59-63, wherein the second VH of P2 comprises the sequence of SEQ ID NO: 26.
65. The protein of any one of embodiments 29-56 and 59-64, wherein the second VL of P4 comprises a sequence having at least 75%, 80%, 85%, 90%, 95%, or 99% sequence identity to the sequence of SEQ ID NO: 27.
66. The protein of any one of embodiments 29-56 and 59-65, wherein the second VL of P4 comprises the sequence of SEQ ID NO: 27.
67. The protein of any one of embodiments 29-54 and 58-62, wherein the second VH of P3 comprises a sequence having at least 75%, 80%, 85%, 90%, 95%, or 99% sequence identity to the sequence of SEQ ID NO: 66.
68. The protein of any one of embodiments 29-54, 58-62, and 67, wherein the second VH of P3 comprises the sequence of SEQ ID NO: 66.
69. The protein of any one of embodiments 29-54, 58-62, 67, and 68, wherein the second VL of P4 comprises a sequence having at least 75%, 80%, 85%, 90%, 95%, or 99% sequence identity to the sequence of SEQ ID NO: 67.
70. The protein of any one of embodiments 29-54, 58-62, and 67-69, wherein the second VL of P4 comprises the sequence of SEQ ID NO: 67.
71. The protein of any one of embodiments 29-56 and 59-66, wherein the first heavy chain comprises:
   (a) the first VH of P1 having at least 75%, 80%, 85%, 90%, 95%, or 99% sequence identity to the sequence of SEQ ID NO: 24; and
   (b) the second VH of P2 having at least 75%, 80%, 85%, 90%, 95%, or 99% sequence identity to the sequence of SEQ ID NO: 26.
72. The protein of any one of embodiments 29-56, 59-66, and 71, wherein the first heavy chain comprises:
   (a) the first VH of P1 comprising the sequence of SEQ ID NO: 24; and
   (b) the second VH of P2 comprising the sequence of SEQ ID NO: 26.
73. The protein of any one of embodiments 29-54, 58-62, and 67-70, wherein the first heavy chain comprises:
   (a) the first VH of P1 having at least 75%, 80%, 85%, 90%, 95%, or 99% sequence identity to the sequence of SEQ ID NO: 24; and
   (b) the second VH of P2 having at least 75%, 80%, 85%, 90%, 95%, or 99% sequence identity to the sequence of SEQ ID NO: 66.
74. The protein of any one of embodiments 29-54, 58-62, 67-70, and 73, wherein the first heavy chain comprises:
   (a) the first VH of P1 comprising the sequence of SEQ ID NO: 24; and
   (b) the second VH of P2 comprising the sequence of SEQ ID NO: 66.
75. The protein of any one of embodiments 29-74, wherein the first light chain comprises:
   (a) the first VL of P3 having at least 75%, 80%, 85%, 90%, 95%, or 99% sequence identity to the sequence of SEQ ID NO: 25; and
   (b) the second VL of P4 having at least 75%, 80%, 85%, 90%, 95%, or 99% sequence identity to the sequence of SEQ ID NO: 27.
76. The protein of any one of embodiments 29-75, wherein the first light chain comprises:
   (a) the first VL of P3 comprising the sequence of SEQ ID NO: 25; and
   (b) the second VL of P4 comprising the sequence of SEQ ID NO: 27.
77. The protein of any one of embodiments 29-74, wherein the first light chain comprises:
   (a) the first VL of P3 having at least 75%, 80%, 85%, 90%, 95%, or 99% sequence identity to the sequence of SEQ ID NO: 25; and
   (b) the second VL of P4 having at least 75%, 80%, 85%, 90%, 95%, or 99% sequence identity to the sequence of SEQ ID NO: 67.
78. The protein of any one of embodiments 29-75, wherein the first light chain comprises:
   (a) the first VL of P3 comprising the sequence of SEQ ID NO: 25; and
   (b) the second VL of P4 comprising the sequence of SEQ ID NO: 67.
79. The protein of any one of embodiments 29-78,
   (a) wherein the first heavy chain comprises:
      (i) the first VH of P1 having at least 75%, 80%, 85%, 90%, 95%, or 99% sequence identity to the sequence of SEQ ID NO: 24; and
      (ii) the second VH of P2 having at least 75%, 80%, 85%, 90%, 95%, or 99% sequence identity to the sequence of SEQ ID NO: 26; and
   (b) wherein the first light chain comprises:
      (i) the first VL of P3 having at least 75%, 80%, 85%, 90%, 95%, or 99% sequence identity to the sequence of SEQ ID NO: 25; and
      (ii) the second VL of P4 having at least 75%, 80%, 85%, 90%, 95%, or 99% sequence identity to the sequence of SEQ ID NO: 27.
80. The protein of any one of embodiments 29-79,
   (a) wherein the first heavy chain comprises:
      (i) the first VH of P1 comprising the sequence of SEQ ID NO: 24; and
      (ii) the second VH of P2 comprising the sequence of SEQ ID NO: 26; and
   (b) wherein the first light chain comprises:
      (i) the first VL of P3 comprising the sequence of SEQ ID NO: 25; and
      (ii) the second VL of P4 comprising the sequence of SEQ ID NO: 27.
81. The protein of any one of embodiments 29-78,
   (a) wherein the first heavy chain comprises:
      (i) the first VH of P1 having at least 75%, 80%, 85%, 90%, 95%, or 99% sequence identity to the sequence of SEQ ID NO: 24; and
      (ii) the second VH of P2 having at least 75%, 80%, 85%, 90%, 95%, or 99% sequence identity to the sequence of SEQ ID NO: 66; and
   (b) wherein the first light chain comprises:
      (i) the first VL of P3 having at least 75%, 80%, 85%, 90%, 95%, or 99% sequence identity to the sequence of SEQ ID NO: 25; and
      (ii) the second VL of P4 having at least 75%, 80%, 85%, 90%, 95%, or 99% sequence identity to the sequence of SEQ ID NO: 67.
82. The protein of any one of embodiments 29-78 and 81,
   (a) wherein the first heavy chain comprises:
      (i) the first VH of P1 comprising the sequence of SEQ ID NO: 24; and
      (ii) the second VH of P2 comprising the sequence of SEQ ID NO: 66; and
   (b) wherein the first light chain comprises:
      (i) the first VL of P3 comprising the sequence of SEQ ID NO: 25; and
      (ii) the second VL of P4 comprising the sequence of SEQ ID NO: 67.
83. The protein of any one of embodiments 29-31 and 33-82, wherein the first CH1 of P1 comprises a sequence having at least 75%, 80%, 85%, 90%, 95%, or 99% sequence identity to the sequence of SEQ ID NO: 5.
84. The protein of any one of embodiments 29-31 and 33-83, wherein the first CH1 of P1 comprises the sequence of SEQ ID NO: 5.
85. The protein of any one of embodiments 29-31 and 33-84, wherein the second CH1 of P2 comprises a sequence having at least 75%, 80%, 85%, 90%, 95%, or 99% sequence identity to the sequence of SEQ ID NO: 5.
86. The protein of any one of embodiments 29-31 and 33-85, wherein the second CH1 of P2 comprises the sequence of SEQ ID NO: 5.
87. The protein of any one of embodiments 29-31 and 35-86, wherein the first CL of P3 is a kappa light chain constant domain.
88. The protein of embodiment 87, wherein the kappa light chain constant domain comprises a sequence having at least 75%, 80%, 85%, 90%, 95%, or 99% sequence identity to the sequence of SEQ ID NO: 7.
89. The protein of embodiment 87 or 88, wherein the kappa light chain constant domain comprises the sequence of SEQ ID NO: 7.
90. The protein of any one of embodiments 29-31 and 38-89, wherein the second CL of P4 is kappa light chain constant domain.
91. The protein of embodiment 90, wherein the kappa light chain constant domain comprises a sequence having at least 75%, 80%, 85%, 90%, 95%, or 99% sequence identity to the sequence of SEQ ID NO: 7.
92. The protein of embodiment 90 or 91, wherein the kappa light chain constant domain comprises the sequence of SEQ ID NO: 7.
93. The protein of any one of embodiments 29-92, wherein the first heavy chain comprises a sequence having at least 75%, 80%, 85%, 90%, 95%, or 99% sequence identity to the sequence of SEQ ID NO: 30, SEQ ID NO: 31, SEQ ID NO: 75, SEQ ID NO: 76, SEQ ID NO: 77, SEQ ID NO: 78, SEQ ID NO: 79, SEQ ID NO: 80, SEQ ID NO: 81, or SEQ ID NO: 82.
94. The protein of any one of embodiments 29-93, wherein the first heavy chain comprises the sequence of SEQ ID NO: 30, SEQ ID NO: 31, SEQ ID NO: 75, SEQ ID NO: 76, SEQ ID NO: 77, SEQ ID NO: 78, SEQ ID NO: 79, SEQ ID NO: 80, SEQ ID NO: 81, or SEQ ID NO: 82.
95. The protein of any one of embodiments 29-94, wherein the first light chain comprises a sequence having at least 75%, 80%, 85%, 90%, 95%, or 99% sequence identity to the sequence of SEQ ID NO: 69, SEQ ID NO: 72, SEQ ID NO: 68, SEQ ID NO: 70, SEQ ID NO: 71, SEQ ID NO: 29, SEQ ID NO: 73, SEQ ID NO: 74, or SEQ ID NO: 28.
96. The protein of any one of embodiments 29-95, wherein the first light chain comprises the sequence of SEQ ID NO: 69, SEQ ID NO: 72, SEQ ID NO: 68, SEQ ID NO: 70, SEQ ID NO: 71, SEQ ID NO: 29, SEQ ID NO: 73, SEQ ID NO: 74, or SEQ ID NO: 28.
97. The protein of any one of embodiments 29-96, wherein the first heavy chain and the first light chain comprise the sequence of:
   SEQ ID NO: 30 and SEQ ID NO: 69, respectively;
   SEQ ID NO: 31 and SEQ ID NO: 29, respectively;
   SEQ ID NO: 75 and SEQ ID NO: 68, respectively;
   SEQ ID NO: 76 and SEQ ID NO: 69, respectively;
   SEQ ID NO: 30 and SEQ ID NO: 70, respectively;
   SEQ ID NO: 77 and SEQ ID NO: 71, respectively;
   SEQ ID NO: 78 and SEQ ID NO: 72, respectively;
   SEQ ID NO: 79 and SEQ ID NO: 73, respectively;
   SEQ ID NO: 77 and SEQ ID NO: 74, respectively;
   SEQ ID NO: 80 and SEQ ID NO: 28, respectively;
   SEQ ID NO: 81 and SEQ ID NO: 68, respectively;
   SEQ ID NO: 82 and SEQ ID NO: 69, respectively; or
   SEQ ID NO: 80 and SEQ ID NO: 70, respectively.
98. The protein of any one of embodiments 46-97, wherein the first dimerization domain comprises a sequence having at least 75%, 80%, 85%, 90%, 95%, or 99% sequence identity to the sequence of SEQ ID NO: 83, SEQ ID NO: 84, or SEQ ID NO: 32.
99. The protein of any one of embodiments 46-98, wherein the first dimerization domain comprises the sequence of SEQ ID NO: 83, SEQ ID NO: 84, or SEQ ID NO: 32.
100. The protein of any one of embodiments 29-99, wherein the first heavy chain comprises one or more heavy chain constant region selected from the group consisting of IgG constant region or functional fragment thereof, IgE constant region or functional fragment thereof, IgM constant region or functional fragment thereof, IgD constant region or functional fragment thereof, IgA constant region or functional fragment thereof, IgY constant region or functional fragment thereof, IgG1 constant region or functional fragment thereof, IgG2 constant region or functional fragment thereof, IgG3 constant region or functional fragment thereof, IgG4 constant region or functional fragment thereof, IgAQ1 constant region or functional fragment thereof, and IgA2 constant region or functional fragment thereof.
101. The protein of any one of embodiments 29-100, wherein the first heavy chain comprises one or more immunologically inert constant region.
102. The protein of any one of embodiments 29-101, wherein the first heavy chain comprises one or more heavy chain constant region selected from the group consisting of wild-type human IgG1 constant region, a human IgG1 constant region comprising the amino acid substitutions L234A, L235A and G237A, a wild-type human IgG2 constant region, a wild-type human IgG4 constant region, and a human IgG4 constant region comprising the amino acid substitution S228P, wherein numbering is according to the EU index as in Kabat.
103. The protein of embodiment 101 or 102, wherein the heavy chain constant region comprises a sequence having at least 75%, 80%, 85%, 90%, 95%, or 99%, or 100% sequence identity to the sequence of SEQ ID NO: 6, SEQ ID NO: 8, SEQ ID NO: 9, SEQ ID NO: 10, SEQ ID NO: 11.
104. The protein of any one of embodiments 29-103, further comprising a second heavy chain.
105. The protein of embodiment 104, wherein the second heavy chain further comprises a second dimerization domain.
106. The protein of embodiment 105, wherein the second dimerization domain comprises a sequence having at least 75%, 80%, 85%, 90%, 95%, or 99% sequence identity to the sequence of SEQ ID NO: 83, SEQ ID NO: 84, or SEQ ID NO: 32.
107. The protein of any embodiment 105 or 106, wherein the second dimerization domain comprises the sequence of SEQ ID NO: 83, SEQ ID NO: 84, or SEQ ID NO: 32.
108. The protein of embodiment 105, wherein the first dimerization domain and the second dimerization domain are dimerized.
109. The protein of any one of embodiments 100-104, wherein the second heavy chain comprises:
   (a) a second P1;
   (b) a third linker, wherein the first linker is cleavable by a protease; and
   (c) a second P2.
110. The protein of embodiment 109, further comprising a second light chain, wherein the second light chain comprises:
   (a) a second P3;
   (b) a fourth linker; and
   (c) a second P4.
111. The protein of any preceding embodiment, wherein the C-terminus of the first heavy chain is linked to the N-terminus of the first dimerization domain and the C-terminus of the first light chain is linked to the N-terminus of the second dimerization domain.
112. A protein comprising the structure of: [P1]-[A]-[P2]-[P3]-[B]-[P4], wherein:
   (a) [P1] comprises a first heavy chain variable domain (VH) and optionally a first heavy chain constant domain 1 (CH1) of a first antigen binding domain;
   (b) [A] comprises a first linker, wherein the first linker is cleavable by a protease;
   (c) [P2] comprises a second VH and optionally a second CH1 of a second antigen binding domain;
   (d) [P3] comprises a first light chain variable domain (VL) and optionally a first light chain constant domain (CL) of the first antigen binding domain;
   (e) [B] comprises a second linker, wherein the second linker is cleavable by a protease;
   (f) [P4] comprises a second VL and optionally a second CL of the second antigen binding domain;
   (g) [P1] and [P3] are covalently linked;
   (h) [P2] and [P4] are covalently linked;
   (i) C-terminus of [P1] is linked to N-terminus of [A];
   (j) C-terminus of [A] is linked to N-terminus of [P2];
   (k) C-terminus of [P3] is linked to N-terminus of [B];
   (l) C-terminus of [B] is linked to N-terminus of [P4];
wherein the first antigen binding domain binds to PD-L1;
wherein when [A] and [B] are cleaved, the second antigen binding domain binds to CD28.

What is claimed is:

1. A protein comprising a first polypeptide chain comprising a heavy chain and a second polypeptide chain comprising a light chain,
wherein the heavy chain comprises, in N-terminus to C-terminus order, an anti-PD-L1 heavy chain variable (VH) domain, a first CH1 domain, a first linker, an anti-CD28 VH domain, and a second CH1 domain;
wherein the light chain comprises, in N-terminus to C-terminus order, an anti-PD-L1 light chain variable (VL) domain, a first immunoglobulin light chain constant region, a second linker, an anti-CD28 VL domain, and a second immunoglobulin light chain constant region.

2. The protein of claim 1, wherein the heavy chain comprises in N-terminus to C-terminus order, the anti-PD-L1 VH domain, the first CH1 domain, the first linker, the anti-CD28 VH domain, the second CH1 domain, a hinge, a CH2 domain, and a CH3 domain.

3. The protein of claim 1, wherein the protein further comprises a third polypeptide chain comprising a hinge and a Fc region, wherein the third polypeptide chain comprises the amino acid sequence of SEQ ID NO: 32.

4. (canceled)

5. The protein of claim 1, wherein the first linker comprises the amino acid sequence of SEQ ID NO: 1, SEQ ID NO: 2, SEQ ID NO: 3, or SEQ ID NO: 4.

6. The protein of claim 1, wherein the second linker comprises the amino acid sequence of SEQ ID NO: 1, SEQ ID NO: 2, SEQ ID NO: 3, or SEQ ID NO: 4.

7. The protein of claim 1,
wherein the anti-PD-L1 VH domain comprises a HCDR1 comprising the amino acid sequence of SEQ ID NO: 12, a HCDR2 comprising the amino acid sequence of SEQ ID NO: 13, and a HCDR3 comprising the amino acid sequence of SEQ ID NO: 14;
wherein the anti-PD-L1 VL domain comprises a LCDR1 comprising the amino acid sequence of SEQ ID NO: 15, a LCDR2 comprising the amino acid sequence of SEQ ID NO: 16, and a LCDR3 comprising the amino acid sequence of SEQ ID NO: 17;
wherein the anti-CD28 VH domain comprises a HCDR1 comprising the amino acid sequence of SEQ ID NO: 18, a HCDR2 comprising the amino acid sequence of SEQ ID NO: 19, and a HCDR3 comprising the amino acid sequence of SEQ ID NO: 20; and
wherein the anti-CD28 VL domain comprises a LCDR1 comprising the amino acid sequence of SEQ ID NO: 21, a LCDR2 comprising the amino acid sequence of SEQ ID NO: 22, and a LCDR3 comprising the amino acid sequence of SEQ ID NO: 23.

8. The protein of claim 1, wherein the anti-PD-L1 VH domain comprises the amino acid sequence of SEQ ID NO: 24, and the anti-PD-L1 VL domain comprises the amino acid sequence of SEQ ID NO: 25.

9. The protein of claim 1, wherein the anti-CD28 VH domain comprises the amino acid sequence of SEQ ID NO: 26, and the anti-CD28 VL domain comprises the amino acid sequence of SEQ ID NO: 27.

10. (canceled)

11. The protein of claim 1, wherein the heavy chain comprises the amino acid sequence of SEQ ID NO: 30 or SEQ ID NO: 31.

12. The protein of claim 1, wherein the light chain comprises the amino acid sequence of SEQ ID NO: 28 or SEQ ID NO: 29.

13. The protein of claim 3, wherein
(a) the heavy chain comprises the amino acid sequence of SEQ ID NO: 30, the light chain comprises the amino acid sequence of SEQ ID NO: 28, and the third polypeptide chain comprises the amino acid sequence of SEQ ID NO: 32; or
(b) the heavy chain comprises the amino acid sequence of SEQ ID NO: 31, the light chain comprises the amino acid sequence of SEQ ID NO: 29, and the third polypeptide chain comprises the amino acid sequence of SEQ ID NO: 32.

14.-17. (canceled)

18. An immunoconjugate comprising the protein of claim 1, linked to a therapeutic agent.

19. (canceled)

20. A pharmaceutical composition comprising the protein of claim 1, and a pharmaceutically acceptable carrier, diluent, or excipient.

21. A nucleic acid molecule encoding
(a) the heavy chain amino acid sequence;
(b) the light chain amino acid sequence; or
(c) both the heavy chain and the light chain amino acid sequences of the protein of claim 1.

22.-25. (canceled)

26. A method for treating cancer in a subject, the method comprising administering to the subject a therapeutically effective amount of the protein of claim 1.

27. (canceled)

28. The method of claim 26, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

29. The protein of claim 1, wherein the anti-PD-L1 VH domain comprises an amino acid sequence having at least 90% sequence identity to the sequence of SEQ ID NO: 24, and the anti-PD-L1 VL domain comprises an amino acid sequence having at least 90% sequence identity to the sequence of SEQ ID NO: 25.

30. The protein of claim 1, wherein the anti-CD28 VH domain comprises an amino acid sequence having at least 90% sequence identity to the sequence of SEQ ID NO: 26, and the anti-CD28 VL domain comprises an amino acid sequence having at least 90% sequence identity to the sequence of SEQ ID NO: 27.


SEQUENCE LISTING (84 sequences)

SEQ ID NO: 1 - protein, 11 aa, synthetic construct
GPAPELLGGGS

SEQ ID NO: 2 - protein, 11 aa, synthetic construct
GPAPLGLGGGS

SEQ ID NO: 3 - protein, 12 aa, synthetic construct
GPAPELLGGGGS

SEQ ID NO: 4 - protein, 12 aa, synthetic construct
GPAPLGLGGGGS

SEQ ID NO: 5 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 330 aa, Homo sapiens [sequence omitted]

SEQ ID NO: 7 - protein, 107 aa, Homo sapiens [sequence omitted]

SEQ ID NO: 8 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 10 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 5 aa, synthetic construct
TYAIS

SEQ ID NO: 13 - protein, 17 aa, synthetic construct
GIIPIFGKAHYAQKFQG

SEQ ID NO: 14 - protein, 14 aa, synthetic construct
KFHFVSGSPFGMDV

SEQ ID NO: 15 - protein, 11 aa, synthetic construct
RASQSVSSYLA

SEQ ID NO: 16 - protein, 7 aa, synthetic construct
DASNRAT

SEQ ID NO: 17 - protein, 8 aa, synthetic construct
QQRSNWPT

SEQ ID NO: 18 - protein, 5 aa, synthetic construct
SYYIH

SEQ ID NO: 19 - protein, 17 aa, synthetic construct
SIYPGNVNTNYNEKEKD

SEQ ID NO: 20 - protein, 11 aa, synthetic construct
SHYGLDWNFDV

SEQ ID NO: 21 - protein, 11 aa, synthetic construct
QASQNIYVWLN

SEQ ID NO: 22 - protein, 7 aa, synthetic construct
KASNLHT

SEQ ID NO: 23 - protein, 9 aa, synthetic construct
QQGQTYPYT

SEQ ID NO: 24 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 25 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 26 - protein, 120 aa, synthetic construct
QVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSS

SEQ ID NO: 27 - protein, 107 aa, synthetic construct
DIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIK

SEQ ID NO: 28 - protein, 438 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 687 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 688 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 223 aa, synthetic construct
SCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 11 aa, synthetic construct
GPAPLGLRGGS

SEQ ID NO: 34 - protein, 7 aa, synthetic construct
GDTFSTY

SEQ ID NO: 35 - protein, 6 aa, synthetic construct
IPIFGK

SEQ ID NO: 36 - protein, 8 aa, synthetic construct
GDTFSTYA

SEQ ID NO: 37 - protein, 8 aa, synthetic construct
IIPIFGKA

SEQ ID NO: 38 - protein, 16 aa, synthetic construct
ARKFHFVSGSPFGMDV

SEQ ID NO: 39 - protein, 6 aa, synthetic construct
QSVSSY

SEQ ID NO: 40 - protein, 5 aa, synthetic construct
SYYIH

SEQ ID NO: 41 - protein, 17 aa, synthetic construct
SIYPGNVNTNYNEKEKD

SEQ ID NO: 42 - protein, 11 aa, synthetic construct
SHYGLDWNFDV

SEQ ID NO: 43 - protein, 7 aa, synthetic construct
GYTFTSY

SEQ ID NO: 44 - protein, 6 aa, synthetic construct
YPGNVN

SEQ ID NO: 45 - protein, 8 aa, synthetic construct
GYTFTSYY

SEQ ID NO: 46 - protein, 8 aa, synthetic construct
IYPGNVNT

SEQ ID NO: 47 - protein, 13 aa, synthetic construct
TRSHYGLDWNFDV

SEQ ID NO: 48 - protein, 11 aa, synthetic construct
QASQNIYVWLN

SEQ ID NO: 49 - protein, 7 aa, synthetic construct
KASNLHT

SEQ ID NO: 50 - protein, 9 aa, synthetic construct
QQGQTYPYT

SEQ ID NO: 51 - protein, 6 aa, synthetic construct
QNIYVW

SEQ ID NO: 52 - skipped

SEQ ID NO: 53 - protein, 10 aa, synthetic construct
GYTFTEYIIH

SEQ ID NO: 54 - protein, 19 aa, synthetic construct
IGWFYPGSNDIQYNAQFKG

SEQ ID NO: 55 - protein, 12 aa, synthetic construct
RDDFSGYDALPY

SEQ ID NO: 56 - protein, 7 aa, synthetic construct
GYTFTEY

SEQ ID NO: 57 - protein, 6 aa, synthetic construct
YPGSND

SEQ ID NO: 58 - protein, 8 aa, synthetic construct
GYTFTEYI

SEQ ID NO: 59 - protein, 8 aa, synthetic construct
FYPGSNDI

SEQ ID NO: 60 - protein, 14 aa, synthetic construct
ARRDDFSGYDALPY

SEQ ID NO: 61 - protein, 11 aa, synthetic construct
KTNENIYSNLA

SEQ ID NO: 62 - protein, 7 aa, synthetic construct
AATHLVE

SEQ ID NO: 63 - protein, 9 aa, synthetic construct
QHFWGTPCT

SEQ ID NO: 64 - protein, 6 aa, synthetic construct
ENIYSN

SEQ ID NO: 65 - skipped

SEQ ID NO: 66 - protein, 121 aa, synthetic construct
QVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 67 - protein, 107 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIK

SEQ ID NO: 68 - protein, 438 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 438 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 438 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 661 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 662 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 661 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 661 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGSDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 688 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 687 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 683 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 684 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 683 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 687 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 688 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 687 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSQVQLVQSGAEVKKPGASVKVSCKASGYTFTSYYIHWVRQAPGQGLEWIGSIYPGNVNTNYNEKEKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 223 aa, synthetic construct
GGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 223 aa, synthetic construct
GGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK